(19) United States
(12) Patent Application Publication
(10) Pub. No.: US 2008/0193573 A1
(43) Pub. Date: Aug. 14, 2008

(54) Extracts and methods comprising curcuma species

(76) Inventors: Robert T. Gow, Naples, FL (US); Dan Li, Singapore (SG); H. Brock Manville, Singapore (SG); George W. Sypert, Naples, FL (US); Randall S. Alberte, Falmouth, ME (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/725,140

(22) Filed: Mar. 16, 2007

Related U.S. Application Data
(60) Provisional application No. 60/783,454, filed on Mar. 17, 2006.
(60) Provisional application No. 60/846,205, filed on Sep. 21, 2006.
(60) Provisional application No. 60/873,405, filed on Dec. 7, 2006.

(51) Int. Cl. A61K 36/9066; A61K 31/715; A61K 38/16; A61K 31/12; A23L 1/28; A61K 31/19; A61P 9/00

(52) U.S. Cl. 424/756; 514/54; 514/12; 514/683; 514/678; 426/655; 514/557

(57) ABSTRACT

The present invention relates to extracts of curcuma species plant material using supercritical CO₂ extraction methods, methods of treating a subject suffering from suffering from amyloid plaque aggregation or fibril formation associated with, for example, Alzheimer's disease, and methods of inhibiting amyloid plaque aggregation or fibril formation in tissue thereof.

### RELATED APPLICATIONS

This application claims the benefit of priority to U.S. Provisional Patent Application Ser. Nos. 60/783,454, filed Mar. 17, 2006, 60/846,205, filed Sep. 21, 2006, and 60/873,405, filed Dec. 7, 2006, which are hereby incorporated by reference in their entirety.

### FIELD OF INVENTION

The invention relates to extracts of curcuma genus, particularly curcuma longa (turmeric) and methods of use and preparation thereof.

### BACKGROUND OF THE INVENTION

Turmeric is the dried, ground rhizome of the herb curcuma longa, a plant within the ginger family (Zingiberaceae) of the genus curcuma native to Southern Asia. In addition to its native habitat, turmeric is heavily cultivated in China, the Caribbean Islands and South American countries. Commonly used as a spice, turmeric has been extensively utilized as a coloring and flavoring agent in curries and mustards and as an ingredient in cosmetics and traditional medications. The phenolic yellowish pigment of turmeric is comprised of curcuminoids, which account for 3-5% of commercially available turmeric powders and 0.34-0.47% of curry powders (1). These naturally occurring antioxidants have been thought to be responsible for the pharmacological activities associated with turmeric (2). However, it has been recently shown that a peptide protein, turmerin, also exhibits powerful antioxidant and cell protective properties and works synergistically with the curcumins in producing desired clinical effects in animals and humans (3). Furthermore, the volatile oil of turmeric contains the turmerones and other beneficial bioactive chemical constituents (4), and the turmeric polysaccharides also have been shown to have potent immune enhancement, anti-inflammatory and anti-cancer activity (5,6).

Although there are a variety of curcuma species within the curcuma genus, the species curcuma longa L. has been shown to have the greatest therapeutic value (7). The source for these therapeutically valuable chemicals is the rhizome (root) of the curcuma plant also termed “turmeric”.

The four principal chemical constituent fractions exhibiting beneficial therapeutic value are: 1. Essential Oil Fraction (EOF) which contains turmerone, ar-turmerone, alpha-turmerone, beta-turmerone, turmeronol A, turmeronol B, curcumene, alpha-curcumene, beta-curcumine, curcumenol, curlone, curdione, alpha-pinene, beta-pinene, cineole, eugenol, limonene, linalool, terpinene, terpineol, etc.; 2) Curcuminoid Fraction (CF) which contains curcumin, tetrahydrocurcumin, demethoxycurcumin, bisdemethoxycurcumin, 3 geometrical isomers of curcumin, and cyclocurcumin: 3. Turmerin Fraction (TF) which contains a polypeptide protein termed turmerin; and 4. Polysaccharide Fraction (PF) which comprises numerous polysaccharide molecules with only a few molecules that have been purified and characterized such as Ukonan A, Ukonan B, Ukonan C, and Ukonan D (5,8).

There are four principal curcuminoids found in the curcuma species: 1) curcumin; 2) tetrahydrocurcumin; 3) demethoxycurcumin; and 4) bisdemethoxycurcumin (9). Four minor curcuminoid constituents have also been isolated (10,11). Curcumin, the principal curcuminoid, and tetrahydrocurcumin, in some applications, appear to be the important active ingredients responsible for the biological activity. Among turmeric species, the concentrations of the major curcuminoids varies substantially: 1) curcumin 40-70%; 2) demethoxycurcumin 16-40%: and 3) bisdemethoxycurcumin 0-30%. Although the major activity of turmeric is anti-inflammatory, it has also been reported to possess powerful antioxidant, anti-allergic, cell protectant, improved wound healing, anti-Alzheimer's disease, anti-cholesterol (LDL), hepatoprotection, enhanced bile acid flow, anti-spasmodic, anti-bacterial, anti-fungal, and anti-neoplastic (cancer) activity as well as improved vitality. A recent research study conducted at Harvard Medical School indicated that curcumin probably possesses anti-HIV activity as well. In addition, Yale University researchers recently published in the scientific journal, Science, that curcumin significantly cut the deaths among mice with the genetic disease, cystic fibrosis.

In addition to the bioactive curcuminoids, the turmerics also contain a water soluble, 5-kD-peptide, turmerin, which has been shown to be a powerful antioxidant, cell protectant, and anti-neoplastic, polysaccharides which have been shown to have strong immune enhancement, anti-inflammatory, and anti-neoplastic activity and essential oils which have been shown to have anti-oxidant, anti-inflammatory, anti-arthritis, anti-spasmodic, analgesis, anti-allergic, cytoprotection, gastroprotection, hepatoprotection, pulmonary protection, anti-asthmatic, nervous system protection, anti-Alzheimer's disease, anti-Parkinson's disease, anti-cancer, anti-mutagenic activity.

Table 1 lists the principal known beneficial biologically active chemical constituent fractions found in C. longa L.

TABLE 1
Biologically Active Chemical Constituents of
Curcuma longa L. (% mass weight)*

| | | |
|---|---|---|
| Essential Oil Fraction | | 3-6 |
| turmerones | (1.0-4.3) | |
| alpha-turmerone | 0.3-0.5 | |
| ar-turmerone | 0.2-0.4 | |
| beta-turmerone | 0.4-0.7 | |
| curcumenol | 0.1-0.2 | |
| alpha-pinene | 0.1-0.5 | |
| eugenol | 0.1-0.2 | |
| limonene | 0.1-0.2 | |
| Curcuminoid Fraction | | |
| Curcuminoids | | 3-5 |
| curcumin | (40-70%) | |
| tetrahydrocurcumin | | |
| demethoxycurcumin | (16-40%) | |
| bisdemethoxycurcumin | (3-30%) | |
| Turmerin Fraction | | |
| Turmerin | | 0.05-0.15 |
| Polysaccharide Fraction | | |
| Polysaccharides | | 0.8-8.3 |
| Ukonan A | 0.02-0.43 | |
| Ukonan B | 0.0005 | |
| Ukonan C | 0.0006 | |

*Based on scientific literature and HerbalScience GC-MS (Gas Chromatography-Mass Spectrometry) and HPLC (High Performance Liquid Chromatography) analysis of curcuma natural feedstock material.

Preclinical and clinical toxicological studies have demonstrated that the turmeric essential oil, the turmeric curcuminoids, turmerin, and turmeric polysaccharides are safe in very large doses over extended periods of time (2, 12-16).

To briefly summarize the therapeutic value of turmeric's chemical constituents, recent research and clinical studies have demonstrated the following therapeutic effects of the various chemicals, chemical fractions, and gross extracts of the curcuma species which include the following: anti-oxidant activity (EOF, CF, TF, Extract) (4,17,18); anti-inflammatory activity (EOF, CF, TF, PF, Extract) (4,19,20); anti-arthritis/anti-rheumatic (EOF, CF, TF, PF, Extract) (19-21); anti-platelet aggregation/anti-thrombotic (EOF, CF, Extract) (23); anti-hypercholesterolemia (EOF, CF, Extract) (1,24); anti-cardiovascular disease (EOF, CF, TF, Extract) (1,4,17,18,22,23-25); anti-allergic (EOF, CF, Extract) (4,19,20,21); anti-chronic pulmonary disease/anti-asthma (EOF, CF, TF, PF, Extract) (4,19,20,22,26); anti-cystic fibrosis (EOF, CF, TF, PF, Extract) (4,19,20,22,26,27); cell protection (EOF, CF, TF, Extract) (4,17,18,28); gastroprotection, hepatoprotection, billiary protection (EOF, CF, Extract (29); nervous system protection (EOF, CF, TF, Extract) (4,17,18,22,23-25); anti-Alzheimer's and Parkinson's disease (EOF, CF, Extract) (30); anti-multiple sclerosis (CF, Extract) (31); anti-cancer and anti-mutagenicity (EOF, CF, TF, PF, Extract) (4,6,13-18,32,38); Immunological enhancement (EOF, PF, Extract) (5,6,33); anti-viral, anti-HIV, anti-bacterial, and anti-fungal (EOF, CF, PF, Extract (5,6,33,34); and improved wound healing (EOF, CF, Extract) (35). Other studies have demonstrated the vital importance of synergistic interactions of the bioactive chemical constituents of the curcuma species (36,37).

### SUMMARY OF THE INVENTION

In one aspect, the present invention relates to a curcuma species extract comprising a fraction having a Direct Analysis in Real Time (DART) mass spectrometry chromatogram of any of FIGS. 9, 10, or 14-78. In a further embodiment, the fraction has a DART mass spectrometry chromatogram of any of FIGS. 14-31, 36, 37, 41, 51, 52, or 56. In a further embodiment, the fraction has a DART mass spectrometry chromatogram of any of FIGS. 35, 38-40, 50, or 53-55. In a further embodiment, the fraction has a DART mass spectrometry chromatogram of any of FIGS. 9, 10, 42-46, or 57-61. In a further embodiment, the fraction has a DART mass spectrometry chromatogram of any of FIGS. 32-34 or 47-49. In a further embodiment, the fraction has a DART mass spectrometry chromatogram of any of FIGS. 63-78. In a further embodiment, the fraction has a DART mass spectrometry chromatogram of FIG. 47 or 62. In a further embodiment, the extract comprises an essential oil fraction having a DART mass spectrometry chromatogram of any of FIGS. 63-78 and a polysaccharide fraction having a DART mass spectrometry chromatogram of any of FIGS. 9, 10, 42-46, or 57-61. In a further embodiment, the extract comprises an essential oil fraction having a DART mass spectrometry chromatogram of any of FIGS. 63-78, a polysaccharide fraction having a DART mass spectrometry chromatogram of any of FIGS. 9, 10, 42-46, or 57-61, and a turmerin fraction having a DART mass spectrometry chromatogram of FIG. 47 or 62.

In a further embodiment, the curcuma species extract of the present invention further comprises a curcuminoid, a turmerone, a polysaccharide, and/or turmerin. In a further embodiment, the curcuminoid is selected from the group consisting of curcumin, tetrahydrocurcumin, demethoxycurcumin, bisdemethoxycurcumin, and combinations thereof. In a further embodiment, the amount of curcuminoid is at least about 75, 80, 85, 90, or 95% by weight. In a further embodiment, the turmerone is selected from the group consisting of alpha-turmerone, ar-turmerone, beta-turmerone, and combinations thereof. In a further embodiment, the amount of turmerone is at least 5, 10, 15, 20, or 25% by weight. In a further embodiment, the amount of turmerin is at least about 5, 10, 15, 20, or 25% by weight. In a further embodiment, the polysaccharide is selected from the group consisting of Ukonan A, Ukonan B, Ukonan C, and a combination thereof. In a further embodiment, the amount of polysaccharide is at least about 5, 10, 15, 20, or 25% by weight.

In another aspect, the present invention relates to a food or medicament comprising the curcuma species extract of the present invention.

In another aspect, the present invention relates to a method for treating a subject for arthritis comprising administering to the subject in need thereof an effective amount of the curcuma species extract of the present invention. In a further embodiment, the curcuma species extract further comprises a synergistic amount of α- and/or β-boswellic acid and/or its C-acetates. In a further embodiment, the subject is a primate, bovine, ovine, equine, procine, rodent, feline, or canine. In a further embodiment, the subject is a human.

In another aspect, the present invention relates to a method of treating a subject suffering from amyloid plaque aggregation or fibril formation comprising administering to the subject in need thereof an effective amount of the curcuma species extract of the present invention. In a further embodiment, the subject is suffering from Alzheimer's disease. In a further embodiment, the subject is a primate, bovine, ovine, equine, procine, rodent, feline, or canine. In a further embodiment, the subject is a human.

In another aspect, the present invention relates to a method of preventing amyloid plaque aggregation or fibril formation in tissue comprising contacting the tissue with an effective amount of the curcuma species extract of the present invention.

In another aspect, the present invention relates to a method of preparing a curcuma species extract having at least one predetermined characteristic comprising: sequentially extracting a curcuma species plant material to yield an essential oil fraction, curcuminoid fraction, polysaccharide fraction, and turmerin fraction by a) extracting a curcuma species plant material by supercritical carbon dioxide extraction to yield the essential oil fraction and a first residue; b) extracting either a curcuma species plant material or the first residue from step a) by supercritical carbon dioxide extraction to yield the curcuminoid fraction and a second residue; c) extracting the second residue from step b) by hot water extraction to yield a polysaccharide solution and then precipitating the polysaccharide with ethanol to yield the polysaccharide fraction and a third residue; and d) separating from the third residue from step c) by column chromatography the turmerin fraction.

In a further embodiment, step a) comprises: 1) loading in an extraction vessel, ground curcuma species plant material; 2) adding carbon dioxide under supercritical conditions; 3) contacting the ground curcuma species plant material and the carbon dioxide for a time; and 4) collecting the essential oil fraction in a collection vessel. In a further embodiment, the supercritical conditions comprise a pressure of from about 250 bar to about 500 bar and a temperature of from about 30° C. to about 80° C. In a further embodiment, extracting conditions for step a) comprise an extraction vessel pressure of from about 250 bar to 500 bar and a temperature of from about 35° C. to about 90° C. and a separator collection vessel pressure of from about 40 bar to about 150 bar and a temperature of from about 20° C. to about 50° C.

In a further embodiment step b) comprises: 1) loading in an extraction vessel, either ground curcuma species plant material or the first residue from step a); 2) adding carbon dioxide under supercritical conditions; 3) contacting the ground curcuma species plant material or first residue from step a) and the carbon dioxide for a time; and 4) collecting the curcuminoid fraction in a fractionation separator collection vessel. In a further embodiment, the extraction conditions for step b) comprise an extraction vessel pressure of from about 350 bar to about 700 bar and a temperature of from about 60° C. to about 95° C. and a separator collection vessel pressure of from about 120 bar to about 220 bar and a temperature of from about 55° C. to about 75° C.

In a further embodiment, step c) comprises: 1) contacting the second residue from step b) with a water solution at about 85° C. to about 100° C. for a time sufficient to extract polysaccharides; 2) separating the solid polysaccharides from the solution by ethanol precipitation; and 3) purifying the polysaccharide fraction using column chromatography.

In a further embodiment, step d) comprises: 1) passing the third residue from step c) through a resin column for separation of high and low molecular weight molecules; and 2) purifying the higher molecular weight effluent solution using a cation exchange resin column to collect the turmerin fraction from the effluent solution.

In another aspect, the present invention relates to a curcuma species extract prepared by the methods of the present invention.

In another aspect, the present invention relates to a curcuma species extract comprising curcumin, tetrahydrocurcumin at 0.1 to 5% by weight of the curcumin, demethoxycurcumin at 10 to 20% by weight of the curcumin, and bisdemethoxycurcumin at 1 to 5% by weight of the curcumin.

In another aspect, the present invention relates to a curcuma species extract comprising curcumin, tetrahydrocurcumin at 0.1 to 5% by weight of the curcumin, demethoxycurcumin at 15 to 25% by weight of the curcumin, and bisdemethoxycurcumin at 1 to 10% by weight of the curcumin.

In another aspect, the present invention relates to a curcuma species extract comprising curcumin, tetrahydrocurcumin at 0.1 to 5% by weight of the curcumin, demethoxycurcumin at 20 to 30% by weight of the curcumin, and bisdemethoxycurcumin at 1 to 10% by weight of the curcumin.

In another aspect, the present invention relates to a curcuma species extract comprising curcumin, demethoxycurcumin at 30 to 40% by weight of the curcumin, and bisdemethoxycurcumin at 5 to 15% by weight of the curcumin.

In another aspect, the present invention relates to a curcuma species extract comprising curcumin, demethoxycurcumin at 45 to 55% by weight of the curcumin, and bisdemethoxycurcumin at 40 to 50% by weight of the curcumin.

In another aspect, the present invention relates to a curcuma species extract comprising curcumin, demethoxycurcumin at 15 to 25% by weight of the curcumin, and bisdemethoxycurcumin at 1 to 10% by weight of the curcumin.

In another aspect, the present invention relates to a curcuma species extract comprising curcumin, tetrahydrocurcumin at 0.1 to 5% by weight of the curcumin, demethoxycurcumin at 20 to 30% by weight of the curcumin, and bisdemethoxycurcumin at 5 to 15% by weight of the curcumin.

An additional embodiment is altered profiles (ratios) by percent mass weight of the chemical constituents of the curcuma species in relation to that found in the native plant material or currently available curcuma species extract products. For example, the essential oil fraction may be increased or decreased in relation to the curcuminoid and/or turmerin and/or polysaccharide concentrations. Similarly, the curcuminoid and/or turmerin and/or polysaccharides may be increased or decreased in relation to the other extract constituent fractions to permit novel constituent chemical profile compositions for specific biological effects.

These embodiments of the present invention, other embodiments, and their features and characteristics, will be apparent from the description, drawings and claims that follow.

### BRIEF DESCRIPTION OF THE DRAWINGS

FIG. 1 depicts an exemplary method for the preparation of the essential oil fraction.

FIG. 2 depicts an exemplary method for carrying out the ethanol leaching extraction.

FIG. 3 depicts an exemplary method for SCCO2 purification of the ethanol extracted curcuminoid fraction.

FIG. 4 depicts an exemplary method for purifying and profiling the curcuminoids.

FIG. 5 depicts an exemplary method for carrying out a water leaching of the residue from the ethanol leaching extraction.

FIG. 6 depicts an exemplary method for the preparation of the polysaccharide fraction.

FIG. 7 depicts an exemplary method for the preparation of the turmerin fraction.

FIG. 8 depicts UV spectra scanning between 200-300 nm for turmerin extraction process.

FIG. 9 depicts a representative DART mass spectrum positive ion mode fingerprint for purified turmeric polysaccharide fraction in accordance with one embodiment of the present invention.

FIG. 10 depicts a representative DART mass spectrum negative ion mode fingerprint for purified turmeric polysaccharide fraction in accordance with one embodiment of the present invention.

FIG. 11 depicts the effects of a curcuma extract on Aβ₁₋₄₂ aggregation as determined with the thioflavin T assay. The Aβ₁₋₄₂ peptide (at 50 μM) was incubated at 37° C. on its own, and also in the presence of the curcuma extract or control compound at different doses as indicated for 72 hours. All experiments were carried out in Tris-HCl buffer (pH 7.4). Data are represented as relative fluorescence units (n=3). One-way ANOVA followed by post-hoc comparison revealed significant differences between the turmeric extract and the control compounds at 10 and 20 μM treatment concentrations (P<0.001, ANOVA).

FIG. 12 depicts the effects of a curcuma extract on Aβ₁₋₄₂ aggregation as determined with the thioflavin T assay. The Aβ₁₋₄₂ peptide (at 50 μM) was incubated at 37° C. on its own, and also in the presence or absence of the turmeric extract or control compound (at 10 μM) for different time points as indicated. Data are represented as relative fluorescence units (n=3). One-way ANOVA followed by post-hoc comparison revealed significant differences between the turmeric extract and the control compounds at 48 and 72 hour-incubation (P<0.001).

FIG. 13 depicts how a turmeric extract treatment inhibits Aβ generation in cultured neuronal cells. Aβ_{1-40, 42} peptides were analyzed in conditioned media from SweAPP N2a cells by ELISA (n=3 for each condition). Data are represented as percentage of Aβ_{1-40, 42} peptides secreted 12 hours after turmeric extract treatment relative to control (untreated). One-way ANOVA followed by post-hoc comparison revealed significant differences between turmeric extract and the control compounds at 5, 10, 20, 40 and 80 μM treatment concentrations (P<0.005).

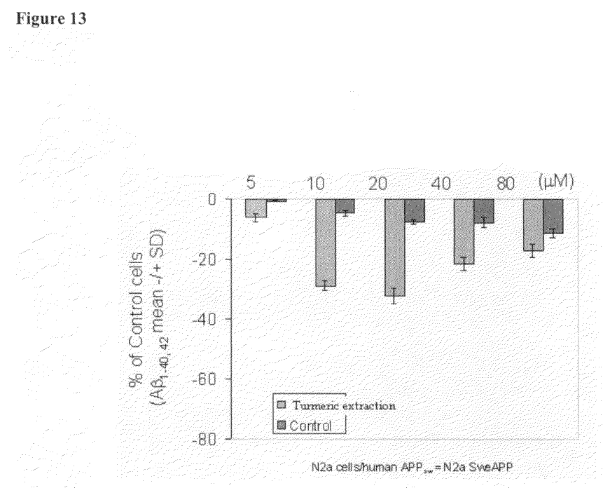

FIG. 14 depicts AccuTOF-DART Mass Spectrum for turmeric extract #139 (positive ion mode). Tetrahydrocurcumin (373.1642)(abund.=0.16), curcumin (369.1332)(abund.=100), demethoxycurcumin (339.1228)(abund.=17.27), and bisdemethoxycurcumin (309.1132)(abund.=2.93) were detected.

FIG. 15 depicts AccuTOF-DART Mass Spectrum for turmeric root extract #310 (positive ion mode). Curcumin (369.1349)(abund.=34.54), demethoxycurcumin (339.1251)(abund.=9.51), and bisdemethoxycurcumin (309.1144)(abund.=5.82) were detected.

FIG. 16 depicts AccuTOF-DART Mass Spectrum for turmeric root extract #311 (positive ion mode).

FIG. 17 depicts AccuTOF-DART Mass Spectrum for turmeric root extract #312 (positive ion mode).

FIG. 18 depicts AccuTOF-DART Mass Spectrum for turmeric root extract #313 (positive ion mode).

FIG. 19 depicts AccuTOF-DART Mass Spectrum for turmeric root extract #314 (positive ion mode). Tetrahydrocurcumin (373.1667)(abund.=0.55), curcumin (369.1345)(abund.=100), demethoxycurcumin (339.1239)(abund.=20.41), and bisdemethoxycurcumin (309.1138)(abund.=5.18) were detected.

FIG. 20 depicts AccuTOF-DART Mass Spectrum for turmeric root extract #315 (positive ion mode). Tetrahydrocurcumin (373.1674)(abund.=0.37), curcumin (369.1358)(abund.=100), demethoxycurcumin (339.1236)(abund.=16.58), and bisdemethoxycurcumin (309.1135)(abund.=3.50) were detected.

FIG. 21 depicts AccuTOF-DART Mass Spectrum for turmeric extract #316 (positive ion mode). Tetrahydrocurcumin (373.1631)(abund.=0.36), curcumin (369.136)(abund.=100), demethoxycurcumin (339.1228)(abund.=22.84), and bisdemethoxycurcumin (309.1122)(abund.=7.59) were detected.

FIG. 22 depicts AccuTOF-DART Mass Spectrum for turmeric extract #317 (positive ion mode). Tetrahydrocurcumin (373.1642)(abund.=0.26), curcumin (369.1343)(abund.=100), demethoxycurcumin (339.1238)(abund.=25.31), and bisdemethoxycurcumin (309.114)(abund.=5.75) were detected.

FIG. 23 depicts AccuTOF-DART Mass Spectrum for turmeric extract #139 (negative ion mode). Curcumin (367.116)(abund.=100), demethoxycurcumin (337.106)(abund.=35.48), and bisdemethoxycurcumin (307.0965)(abund.=9.02) were detected.

FIG. 24 depicts AccuTOF-DART Mass Spectrum for turmeric root extract #310 (negative ion mode). Curcumin (367.1127)(abund.=100), demethoxycurcumin (337.1033)(abund.=50.06), and bisdemethoxycurcumin (307.0942)(abund.=44.26) were detected.

FIG. 25 depicts AccuTOF-DART Mass Spectrum for turmeric root extract #311 (negative ion mode). Curcumin (367.1127)(abund.=100), demethoxycurcumin (337.1033)(abund.=49.82), and bisdemethoxycurcumin (307.0941)(abund.=44.04) were detected.

FIG. 26 depicts AccuTOF-DART Mass Spectrum for turmeric root extract #312 (negative ion mode). Curcumin (367.113)(abund.=100), demethoxycurcumin (337.104)(abund.=18.62), and bisdemethoxycurcumin (307.099)(abund.=3.08) were detected.

FIG. 27 depicts AccuTOF-DART Mass Spectrum for turmeric root extract #313 (negative ion mode). Curcumin (367.1133)(abund.=100), demethoxycurcumin (337.1041)(abund.=19.56), and bisdemethoxycurcumin (307.0976)(abund.=3.75) were detected.

FIG. 28 depicts AccuTOF-DART Mass Spectrum for turmeric root extract #314 (negative ion mode). Curcumin (367.1133)(abund.=100), demethoxycurcumin (337.1042)(abund.=19.71), and bisdemethoxycurcumin (307.0982)(abund.=3.98) were detected.

FIG. 29 depicts AccuTOF-DART Mass Spectrum for turmeric root extract #315 (negative ion mode). Curcumin (367.1128)(abund.=100), demethoxycurcumin (337.1036)(abund.=26.32), and bisdemethoxycurcumin (307.0953)(abund.=8.38) were detected.

FIG. 30 depicts AccuTOF-DART Mass Spectrum for turmeric extract #316 (negative ion mode). Tetrahydrocurcumin (371.1306)(abund.=0.99), curcumin (367.1131)(abund.=100), demethoxycurcumin (337.1043)(abund.=26.54), and bisdemethoxycurcumin (307.0958)(abund.=9.48) were detected.

FIG. 31 depicts AccuTOF-DART Mass Spectrum for turmeric extract #317 (negative ion mode). Curcumin (367.1128)(abund.=100), demethoxycurcumin (337.1035)(abund.=35.48), and bisdemethoxycurcumin (307.0948)(abund.=8.43) were detected.

FIG. 32 depicts AccuTOF-DART Mass Spectrum for turmeric extract with a 75% EtOH solution (HS#136) (positive ion mode). Tetrahydrocurcumin (373.1678)(abund.=0.41), curcumin (369.1418)(abund.=100), demethoxycurcumin (339.1304)(abund.=22.00), and bisdemethoxycurcumin (309.1201)(abund.=5.36) were detected.

FIG. 33 depicts AccuTOF-DART Mass Spectrum for turmeric extract with a 80% EtOH solution (HS#137) (positive ion mode). Tetrahydrocurcumin (373.1655)(abund.=1.09), curcumin (369.1330)(abund.=100), demethoxycurcumin (339.124)(abund.=18.04), and bisdemethoxycurcumin (309.1131)(abund.=5.10) were detected.

FIG. 34 depicts AccuTOF-DART Mass Spectrum for turmeric extract with a 85% EtOH solution (HS#138) (positive ion mode). Tetrahydrocurcumin (373.1722)(abund.=0.31), curcumin (369.1365)(abund.=100), demethoxycurcumin (339.1254)(abund.=13.14), and bisdemethoxycurcumin (309.1156)(abund.=2.48) were detected.

FIG. 35 depicts AccuTOF-DART Mass Spectrum for commercially available (Hara Spices) turmeric root (HS#160) (positive ion mode). Curcumin (369.132)(abund.=0.31) was detected.

FIG. 36 depicts AccuTOF-DART Mass Spectrum for turmeric root from China (HS#161) (positive ion mode). Curcumin (369.1273)(abund.=1.20) was detected.

FIG. 37 depicts AccuTOF-DART Mass Spectrum for turmeric root from India (HS#162) (positive ion mode). Curcumin (369.1335)(abund.=0.54) was detected.

FIG. 38 depicts AccuTOF-DART Mass Spectrum for commercially available (Singapore Tai' Eng) turmeric root (HS#163) (positive ion mode). Curcumin (369.132)(abund.=20.80), demethoxycurcumin (339.12)(abund.=4.83), and bisdemethoxycurcumin (309.111)(abund.=2.05) were detected.

FIG. 39 depicts AccuTOF-DART Mass Spectrum for commercially available (Singapore Tai' Eng) turmeric root (HS#164) (positive ion mode). Curcumin (369.134)(abund.=11.02), demethoxycurcumin (339.1205)(abund.=2.18), and bisdemethoxycurcumin (309.1122)(abund.=1.46) were detected.

FIG. 40 depicts AccuTOF-DART Mass Spectrum for commercially available (Suan Farms) turmeric (HS#165) (positive ion mode). Tetrahydrocurcumin (373.1658)(abund.=0.28), curcumin (369.1331)(abund.=100), demethoxycurcumin (339.1221)(abund.=16.26), and bisdemethoxycurcumin (309.116)(abund.=2.65) were detected.

FIG. 41 depicts AccuTOF-DART Mass Spectrum for turmeric root from Naples (HS#166) (positive ion mode). Curcumin (369.1345)(abund.=3.94), demethoxycurcumin (339.1198)(abund.=0.35), and bisdemethoxycurcumin (309.1106)(abund.=0.14) were detected.

FIG. 42 depicts AccuTOF-DART Mass Spectrum for polysaccharides precipitated by a 20% EtOH solution from an extraction of commercially available turmeric (Hara Spice) (HS#302) (positive ion mode).

FIG. 43 depicts AccuTOF-DART Mass Spectrum for polysaccharides precipitated by a 40% EtOH solution from an extraction of commercially available turmeric (Hara Spice) (HS#303) (positive ion mode).

FIG. 44 depicts AccuTOF-DART Mass Spectrum for polysaccharides precipitated by a 20% EtOH solution from an extraction of commercially available turmeric (Hara Spice) (HS#304) (positive ion mode).

FIG. 45 depicts AccuTOF-DART Mass Spectrum for polysaccharides precipitated by a 80% EtOH solution from an extraction of commercially available turmeric (Hara Spice) (HS#305) (positive ion mode).

FIG. 46 depicts AccuTOF-DART Mass Spectrum for polysaccharides precipitated by a 95% EtOH solution from an extraction of commercially available turmeric (Hara Spice) (HS#306) (positive ion mode). Curcumin (369.1431)(abund.=3.66), demethoxycurcumin (339.1436)(abund.=0.73), and bisdemethoxycurcumin (309.1187)(abund.=0.97) were detected.

FIG. 47 depicts AccuTOF-DART Mass Spectrum for polypeptide turmerin processed from 60% supernatant from an extraction of commercially available turmeric (Hara Spice) (HS#307) (positive ion mode).

FIG. 48 depicts AccuTOF-DART Mass Spectrum for a 75% EtOH extraction of turmeric (HS#136) (negative ion mode). Curcumin (367.1123)(abund.=100), demethoxycurcumin (337.1028)(abund.=42.60), and bisdemethoxycurcumin (307.0941)(abund.=14.09) were detected.

FIG. 49 depicts AccuTOF-DART Mass Spectrum for a 85% EtOH extraction of turmeric (HS#138) (negative ion mode). Curcumin (367.1117)(abund.=100), demethoxycurcumin (337.103)(abund.=23.61), and bisdemethoxycurcumin (307.0953)(abund.=5.46) were detected.

FIG. 50 depicts AccuTOF-DART Mass Spectrum for commercially available (Hara Spices) turmeric root (HS#160) (negative ion mode). Curcumin (367.1125)(abund.=100), demethoxycurcumin (337.1033)(abund.=33.89), and bisdemethoxycurcumin (307.0940)(abund.=19.46) were detected.

FIG. 51 depicts AccuTOF-DART Mass Spectrum for turmeric root from China (HS#161) (negative ion mode). Tetrahydrocurcumin (371.1335)(abund.=4.34), curcumin (367.1126)(abund.=100), demethoxycurcumin (337.1035)(abund.=90.31), and bisdemethoxycurcumin (307.0943)(abund.=39.58) were detected.

FIG. 52 depicts AccuTOF-DART Mass Spectrum for turmeric root from India (HS#162) (negative ion mode). Curcumin (367.1129)(abund.=0.16), demethoxycurcumin (337.1041), and bisdemethoxycurcumin (307.0944) were detected.

FIG. 53 depicts AccuTOF-DART Mass Spectrum for commercially available (Singapore Tai' Eng) turmeric root (HS#163) (negative ion mode). Curcumin (367.1142), demethoxycurcumin (337.1052), and bisdemethoxycurcumin (307.0963) were detected.

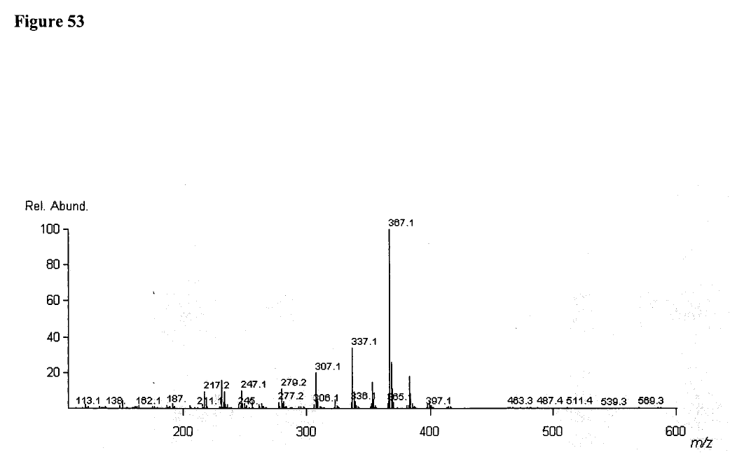

FIG. 54 depicts AccuTOF-DART Mass Spectrum for commercially available (Singapore Tai' Eng) turmeric root (HS#164) (negative ion mode). Curcumin (367.1147), demethoxycurcumin (337.1059), and bisdemethoxycurcumin (307.095) were detected.

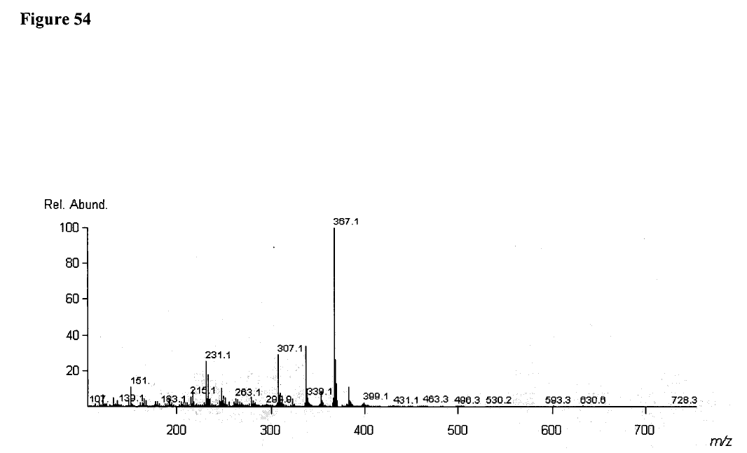

FIG. 55 depicts AccuTOF-DART Mass Spectrum for commercially available (Suan Farms) turmeric (HS#165) (negative ion mode). Tetrahydrocurcumin (371.1282), curcumin (367.1151), demethoxycurcumin (337.1061), and bisdemethoxycurcumin (307.0981) were detected.

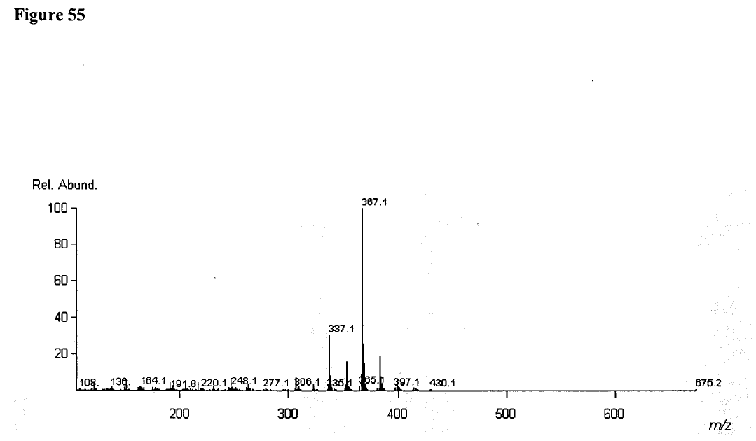

FIG. 56 depicts AccuTOF-DART Mass Spectrum for turmeric root from Naples (HS#166) (negative ion mode). Curcumin (367.1152), demethoxycurcumin (337.1064), and bisdemethoxycurcumin (307.0966) were detected.

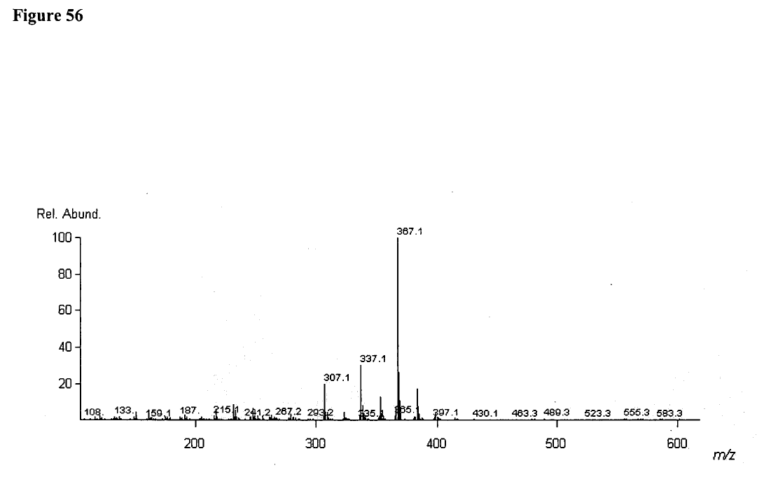

FIG. 57 depicts AccuTOF-DART Mass Spectrum for polysaccharides precipitated by a 20% EtOH solution from an extraction of commercially available turmeric (Hara Spice) (HS#302) (negative ion mode).

FIG. 58 depicts AccuTOF-DART Mass Spectrum for polysaccharides precipitated by a 40% EtOH solution from an extraction of commercially available turmeric (Hara Spice) (HS#303) (negative ion mode).

FIG. 59 depicts AccuTOF-DART Mass Spectrum for polysaccharides precipitated by a 60% EtOH solution from an extraction of commercially available turmeric (Hara Spice) (HS#304) (negative ion mode).

FIG. 60 depicts AccuTOF-DART Mass Spectrum for polysaccharides precipitated by a 80% EtOH solution from an extraction of commercially available turmeric (Hara Spice) (HS#305) (negative ion mode). Curcumin (367.1107) and demethoxycurcumin (337.1114) were detected.

FIG. 61 depicts AccuTOF-DART Mass Spectrum for polysaccharides precipitated by a 95% EtOH solution from an extraction of commercially available turmeric (Hara Spice) (HS#306) (negative ion mode). Curcumin (367.1141) was detected.

FIG. 62 depicts AccuTOF-DART Mass Spectrum for polypeptide turmerin processed from 60% supernatant from an extraction of commercially available turmeric (Hara Spice) (HS#307) (negative ion mode). Curcumin (367.1163) was detected.

FIG. 63 depicts AccuTOF-DART Mass Spectrum for the essential oil fraction from CO₂ supercritical extraction of turmeric at 40° C. and 80 bar (HS#160) (positive ion mode).

FIG. 64 depicts AccuTOF-DART Mass Spectrum for the essential oil fraction from CO₂ supercritical extraction of turmeric at 40° C. and 300 bar (HS#160) (positive ion mode).

FIG. 65 depicts AccuTOF-DART Mass Spectrum for the essential oil fraction from CO₂ supercritical extraction of turmeric at 40° C. and 500 bar (HS#160) (positive ion mode).

FIG. 66 depicts AccuTOF-DART Mass Spectrum for the essential oil fraction from CO₂ supercritical extraction of turmeric at 60° C. and 100 bar (HS#160) (positive ion mode).

FIG. 67 depicts AccuTOF-DART Mass Spectrum for the essential oil fraction from CO₂ supercritical extraction of turmeric at 60° C. and 300 bar (HS#160) (positive ion mode).

FIG. 68 depicts AccuTOF-DART Mass Spectrum for the essential oil fraction from CO₂ supercritical extraction of turmeric at 80° C. and 100 bar (HS#160) (positive ion mode).

FIG. 69 depicts AccuTOF-DART Mass Spectrum for the essential oil fraction from CO₂ supercritical extraction of turmeric at 80° C. and 300 bar (HS#160) (positive ion mode).

FIG. 70 depicts AccuTOF-DART Mass Spectrum for the essential oil fraction from CO₂ supercritical extraction of turmeric at 40° C. and 500 bar (HS#164) (positive ion mode).

FIG. 71 depicts AccuTOF-DART Mass Spectrum for the essential oil fraction from CO₂ supercritical extraction of turmeric at 40° C. and 80 bar (HS#160) (negative ion mode).

FIG. 72 depicts AccuTOF-DART Mass Spectrum for the essential oil fraction from CO₂ supercritical extraction of turmeric at 40° C. and 300 bar (HS#160) (negative ion mode).

FIG. 73 depicts AccuTOF-DART Mass Spectrum for the essential oil fraction from CO₂ supercritical extraction of turmeric at 40° C. and 500 bar (HS#160) (negative ion mode).

FIG. 74 depicts AccuTOF-DART Mass Spectrum for the essential oil fraction from CO₂ supercritical extraction of turmeric at 60° C. and 100 bar (HS#160) (negative ion mode).

FIG. 75 depicts AccuTOF-DART Mass Spectrum for the essential oil fraction from CO₂ supercritical extraction of turmeric at 60° C. and 300 bar (HS#160) (negative ion mode).

FIG. 76 depicts AccuTOF-DART Mass Spectrum for the essential oil fraction from CO₂ supercritical extraction of turmeric at 80° C. and 100 bar (HS#160) (negative ion mode).

FIG. 77 depicts AccuTOF-DART Mass Spectrum for the essential oil fraction from CO₂ supercritical extraction of turmeric at 80° C. and 300 bar (HS#160) (negative ion mode).

FIG. 78 depicts AccuTOF-DART Mass Spectrum for the essential oil fraction from CO₂ supercritical extraction of turmeric at 40° C. and 500 bar (HS#164) (negative ion mode).

FIG. 79 depicts the chemical structures of curcumin, tetrahydrocurcumin, demethoxycurcumin, and bisdemethoxycurcumin, which together form a group of compounds referred to herein as “curcuminoids.”

FIG. 80 depicts the chemical structures of some of the compounds found in the essential oil fraction of the curcuma extractions.

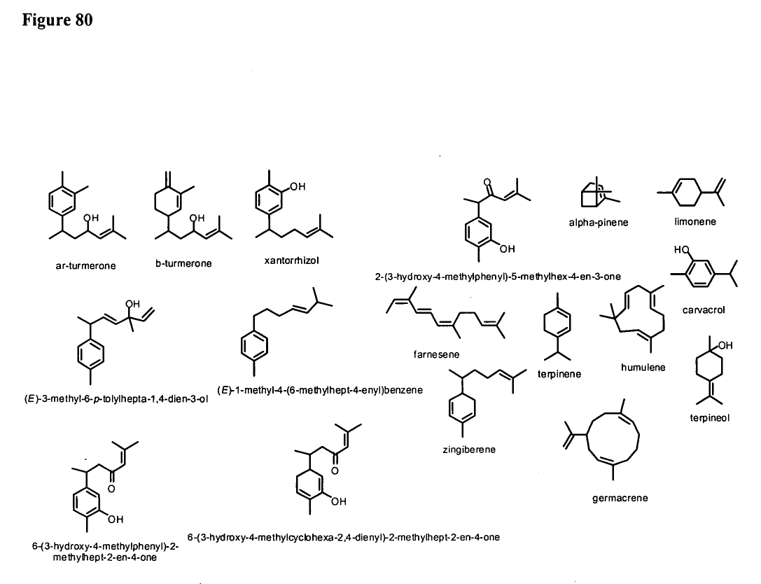

### DETAILED DESCRIPTION OF THE INVENTION

The present invention features extracts of curcuma species and related species such as, but not limited to, curcuma longa L. As used herein, curcuma refers to the plant or plant material derived from the plant Zingiberaceae family, herein the genus includes, but is not limited to, C. longa L, C. aromatica Salisb., C. amada Roxb., C. zeodaria Rosc., and C. xanthorrhizia Roxb. The term includes all clones, cultivars, variants, and sports of curcuma and related species.

### DEFINITIONS

The articles “a” and “an” are used herein to refer to one or to more than one (i.e. to at least one) of the grammatical object of the article. By way of example, “an element” means one element or more than one element.

As known in the art, the term “compound” does not mean one molecule, but multiples or moles of molecules on one or more compounds. In addition, as known in the art, the term “compound” means a chemical constituent possessing distinct chemical and physical properties, whereas “compounds” refer to more than one chemical constituent compound.

The terms “comprise” and “comprising” are used in the inclusive, open sense, meaning that additional elements may be included.

The term “consisting” is used to limit the elements to those specified except for impurities ordinarily associated therewith.

The term “consisting essentially of” is used to limit the elements to those specified and those that do not materially affect the basic and novel characteristics of the material or steps.

The term “curcuma” is also used interchangeably with “turmeric” and includes plants, clones, variants, and sports from the plant Zingiberaceae family.

As used herein, the term “curcuma constituents” or “turmeric constituents” shall mean chemical compounds found in the curcuma species and shall include all such chemical compounds identified above as well as other chemical compounds found in curcuma species, including, but not limited to, turmerones, curcuminoids, turmerin, and polysaccharides.

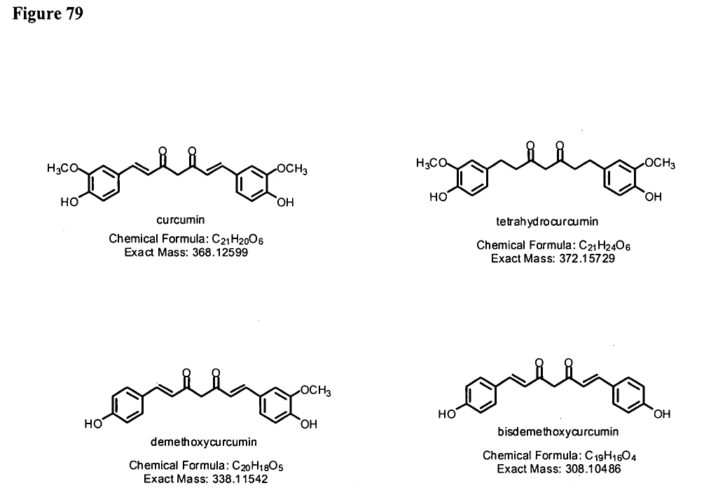

As used herein, the term “curcumin” refers to one component of the curcuminoids. Its structure is depicted in FIG. 79.

As used herein, the term “curcuminoid fraction” comprises the water insoluble, ethanol soluble compounds obtained or derived from curcuma and related species including the chemical compounds classified as curcuminoids. Components of the curcuminoids include curcumin, tetrahydrocurcumin, demethoxycurcumin, and bisdemethoxycurcumin, and are depicted in FIG. 79.

The term “effective amount” as used herein refers to the amount necessary to elicit the desired biological response. As will be appreciated by those of ordinary skill in this art, the effective amount of a composite or bioactive agent may vary depending on such factors as the desired biological endpoint, the bioactive agent to be delivered, the composition of the encapsulating matrix, the target tissue, etc.

As used herein, the term “essential oil fraction” comprises lipid soluble, water insoluble compounds obtained or derived from curcuma and related species including the chemical compounds classified as turmerones.

As used herein, the term “feedstock” generally refers to raw plant material, comprising leaves, branches, rhizomes, roots, including, but not limited to main roots, tail roots, and fiber roots, stems, leaves, seeds, and flowers, wherein the plant or constituent parts may comprise material that is raw, dried, steamed, heated, or otherwise processed to affected the size and integrity of the plant material. Occasionally, the term “feedstock” may be used to characterize an extraction product that is to be used as a feed source for additional extraction processes.

As used herein, the term “fraction” means the extraction composition comprising a specific group of chemical compounds characterized by certain physical, chemical properties or physical or chemical properties. For example, the essential oil fraction (EOF) contains the turmerones as well as other chemical constituents, the curcuminoid fraction contains the curcuminoids as well as other ethanol soluble chemical constituents, the turmerin fraction contains turmerin as well as other small water soluble protein chemical constituents, and the polysaccharide fraction contains ukonan A, B, C, and D as well as other polysaccharides of various molecular weight. Other chemical constituents of curcuma and related species may also be present in these extraction fractions.

As used herein, the term “one or more compounds” means that at least one compound, such as turmerone (an essential oil turmeric chemical constituent), curcumin (a water insoluble, ethanol insoluble diferuloylmethane turmeric chemical constituent), turmerin (a water soluble peptide protein), and ukonan A (a water soluble, ethanol insoluble polysaccharide chemical constituent) is intended, or that more than one compound is, for example, curcumin and turmerin is intended.

As used herein, the term “polysaccharide fraction” comprises the water soluble, ethanol insoluble compounds obtained from curcuma and related species including the chemical compounds classified as ukonans.

As used herein, the term “profile” refers to the ratios by percent mass weight of the chemical compounds within an extraction fraction or to the ratios of the percent mass weight of each of the four curcuma fraction chemical constituents in a final curcuma extraction.

As used herein, the term “purified” fractions or extractions means a fraction or composition comprising a specific group of chemical constituents characterized by certain physical or chemical properties that are concentrated to greater than 70% of the fraction's or extraction's chemical constituents by % dry mass weight. In other words, a purified fraction or extraction comprises less than 30% dry mass weight of chemical constituents that are not characterized by certain desired physical, chemical properties or physical or chemical properties that define the fraction or extraction.

As used herein, the term “rhizome” refers to the constituent part of curcuma and related species comprising a horizontal or vertical root stems or modified stems (e.g., tubers), which may be in part or in whole, underground, further comprising shoots above or roots below, including, but not limited to, primary roots, secondary roots, and tertiary roots.

The term “synergistic” is art recognized and refers to two or more components working together so that the total effect is greater than the sum of the components.

The term “treating” is art-recognized and refers to curing as well as ameliorating at least one symptom of any condition or disorder.

As used herein, the term “tumerin fraction” comprises the water and ethanol soluble compounds obtained or derived from curcuma and related species including the chemical compound classified as turmerin, a peptide.

### Extractions

### Essential Oil Fraction

Extractions of the present invention comprise combinations of one or more curcuma species taught herein. An embodiment of an extraction comprises an essential oil fraction having the components as shown by GC-MS of Table 2.

Turmeric root essential oil fraction was extracted by supercritical carbon dioxide extraction technology by single stage processing. The optimum extraction conditions are at temperatures of 40-60° C. and pressures of 100-300 bar with a yield of ˜3.5%. The major essential oil compounds in Turmeric root are sesquiterpenoids, such as Ar-turmerone, turmerone and curlone and sesquiterpenes, such as curcumene and zingiberene. The essential oil obtained by SCCO2 single stage extraction has high purity of 99% when extracted at temperatures of 40-60° C. and a pressure of 300 bar. Turmerone and curlone are the major compounds, constituting 75%-81% of the total essential oil. Sesquiterpenes constitute 5.6-9.7% of the essential oil, in which curcumene and zingiberene are relatively majors ones. The aforementioned compounds constitute 85-89% of total turmeric essential oil.

In addition, curcuminoid purity is below 2.5% in single stage of SCCO2 extractions at certain conditions, such as T=40 and 60° C. and pressure of 100-500 bar. These conditions can be chosen to extract high purity essential oil from turmeric root.

TABLE 2
Major compounds indentified in curcuma essential oil.

| Peak # | Library/ID | CAS# | Structure | Formula | Mw |
|---|---|---|---|---|---|
| 1 | _-Curcumene | 644-30-4 | 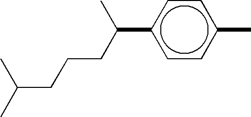 | C₁₅H₂₂ | 202 |
| 2 | (-)-Zingiberene | 495-60-3 | 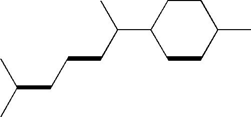 | C₁₅H₂₄ | 204 |
| 3 | _-Sesquiphellandrene | 20307-83-9 | 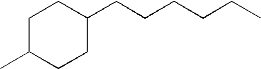 | C₁₅H₂₄ | 204 |
| 4 | Benzene, 1-methyl-4-(1-methylethyl)- | 99-87-6 | 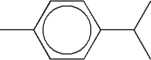 | C₁₀H₁₄ | 134 |
| 5 | Benzene, 1-methyl-2-(1-methylethyl)-; | 527084-4 | 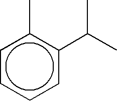 | C₁₀H₁₄ | 134 |
| 6 | Benzene, 4-ethyl-l,2-dimethyl- | 934-80-5 | 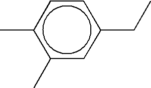 | C₁₀H₁₄ | 134 |
| 7 | Cyclohexene, 1-(1-propynyl)- | 1655-05-6 | | | |
| 8 | ar-tumerone | 532-65-0 | 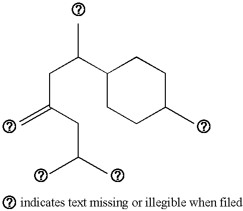 | C₁₅H₂₂O | 218 |
| 9 | β-tumerone | 82508-14-3 | 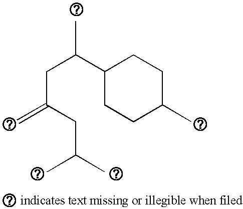 | C₁₅H₂₂O | 218 |
| 10 | Compound 1 | | | | 216 |
| 11 | α-tumerone | 82508-15-4 | 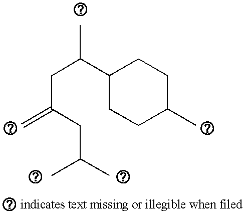 | C₁₅H₂₀O | 216 |
| 12 | (6S,1′R)-6-(1′5′-dimethylenex-4′-enyl)-3-methylcyclohex-2-enone | 72441-71-5 | | | 220 |
| 13 | Compound 2 | | | | 216 |
| 14 | (+)-beta-atlantone | | | | 234 |
| 15 | Compound 3 | | | | 232 |
| 16 | Compound 4 | | | | 218 |
| 17 | Compound 5 | | | | 218 |
| 18 | (+)-alpha-atlantone | | | | 218 |
| 19 | Compound 6 | | | | 218 |
| 20 | 3-buten-2-one, 4-(4-hydroxy-3-methoxyphenyl)- | 1080-122 | 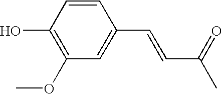 | C₁₁H₁₂O3 | 192 |
| 21 | Compound 7 | | | | 232 |
| 22 | Compound 8 | | | | 234 |
| 23 | Compound 9 | | | | 230 |
| 24 | Hexdecanoic acid,methyl ester | 112-39-0 | 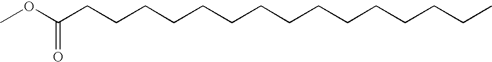 | C₁₇H₃₄O₂ | 270 |
| 25 | Pentadecanoic acid, 14-methyl-, methyl ester | 5129-60-2 | 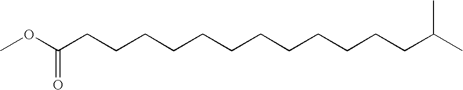 | C₁₇H₃₄O₂ | 270 |
| 26 | 9,12-Octadecadienoicacid, methyl ester, (E,E)- | 2566-97-4 | 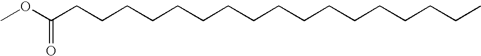 | C₁₉H₃₄O₂ | 294 |

The compounds have retention time peaks of about 30.36 (-curcumene), 30.74 ((−)-zingiberene, 31.68 (-sesquiphellandrene), 33.45 (benzene, 1-methyl-4-(1-methylethyl-), 34.20 (benzene, 1-methyl-2-(1-methylethyl)-), 34.90 (benzene, 4-ethyl-1,2-dimethyl-), 35.21 (cyclohexene, 1-(1-propynyl)-), 35.96 (ar-tumerone), 36.43 (β-tumerone), 37.00 (compound U1), 37.36 α-tumerone), 38.32 ((6S,1′R)-6-(1′5′-dimethylenex-4′-enyl)-3-methylcyclohex-2-enone), 38.57 (compound U2), 38.73 ((+)-beta-atlantone), 38.84 (compound U3), 38.94 (compound U4), 39.24 (compound U5), 39.41 ((+)-alpha-atlantone), 39.64 (compound U6), 39.76 (3-buten-2-one,4-(4-hydroxy-3-methoxyphenyl)-), 40.03 (compound U7), 40.73 (compound U8), 41.02 (compound U9), 41.43 (hexdecanoic acid, methyl ester), 42.05 (pentadecanoic acid, 14-methyl-, methyl ester), and 42.89 (9,12-octadecdienoic acid, methyl ester, (E,E)-) minutes using the GC-MS analytical methods as taught in the present invention.

### Curcuminoid Fraction

Turmeric curcuminoid fractions were extracted and purified by supercritical extraction/fractionation technology with ethanol as the co-solvent. The curcuminoid extraction yields were in the range of 0.74-2.10% with adding 1.2%-3.7% of ethanol as the co-solvent. The curcuminod extraction yield by using pure CO2 was only as highest as 0.27%. Therefore, it is necessary to use ethanol as a co-solvent to increase curcuminoid extraction yield. 70% of the curcuminoids in feedstock have been extracted by adding 3.7% ethanol as co-solvent. The higher the ethanol concentration, the higher the extraction yield. However, it is not good to further increase the ethanol concentration in order to maximize the selectivity of SCCO2 for the curcuminoids.

The extraction conditions were at a temperature of above 80° C. and a pressure above 500 bar. The three separators conditions were at 60-67° C./150-170 bar; 56° C./130 bar and 28.6° C./60 bar, respectively. The target curucminoids are precipitated in the 1^{st} separator. In addition, different operational methods were tested, such as A: Use of three separators continuously during whole processing; B: Two stage process with 1^{st} stage to remove essential oil at mild conditions by only using 3^{rd} separator and 2^{nd} stage to extract and fractionation the curcuminoids by using three separators continuously; and C: Two stage process with 1^{st} stage to remove essential oil. at harsh conditions by using 2^{nd} and 3^{rd} separator and 2^{nd} stage to extract and fractionation curcuminoids by using 1st and 3^{rd} separators. The summarized curcuminoid purity data is shown in Table 3:

TABLE 3
The purity of curcuminoids by SCCO2 extraction/fractionation process.
(A)

| | | | Operation | | | | | | | |
|---|---|---|---|---|---|---|---|---|---|---|
| | | | conditions | | | | | | | |
| | | | | | | | | | T = 67.1 C., | |
| | | | T = 67.1 C., P = 150 bar, | | | | | | P = 160 bar, | |
| | | | density = 0.573 g/cc | | | | | | density = 0.617 g/cc | |
| | | | Operation methods | | | | | | | |
| | | Me—OH | A | A | B | C | C | C | A | A |
| Compound | Feedstock | extracts | Purity (%) | | | | | | | |
| BDMC | 0.61 | 6.10 | 5.4 | 7.0 | 7.3 | 7.4 | 6.3 | 6.3 | 4.2 | 4.2 |
| DMC | 0.43 | 4.20 | 8.0 | 8.7 | 9.7 | 9.6 | 9.7 | 9.2 | 6.9 | 7.3 |
| C | 2.00 | 19.50 | 45.0 | 52.9 | 63.5 | 57.2 | 58.2 | 56.7 | 51.6 | 54.0 |
| Total | 3.04 | 29.80 | 58.5 | 68.7 | 80.5 | 74.2 | 74.2 | 72.3 | 62.7 | 65.5 |

(B)

| Operation | | | | | |
|---|---|---|---|---|---|
| conditions | | | | | |
| T = 60.0 C., P = 130 bar, | T = 61.5 C., P = 150 bar, | | | | |
| density = 0.550 g/cc | density = 0.600 | | | | |
| Operation | | | | | |
| methods | | | | | |
| A | A | B | C | C | C |

| | |
|---|---|
| Compound | Purity (%) |

| | | | | | | |
|---|---|---|---|---|---|---|
| BDMC | 15.9 | 7.5 | 8.2 | 8.5 | 8.1 | 10.3 |
| DMC | 9.5 | 10.6 | 11.5 | 14.8 | 14.0 | 13.2 |
| C | 41.2 | 52.7 | 54.8 | 60.7 | 62.6 | 63.6 |
| Total | 66.6 | 70.8 | 74.4 | 84.0 | 84.7 | 87.1 |

The purity of total curcuminoids can be increased to different levels as follows: greater than 55%, 60%-70%, 70%-80% and greater than 80%, depending on the operational methods. Higher purity was obtained by using two stages processing with 1^{st} stage to remove essential oil and 2^{nd} stage to extract curcuminoids with CO2 and ethanol cosolvent (method C). The summarized curcuminoid profile is shown in Table 4.

TABLE 4
The profile of curcuminoids by SCCO2 extraction/fractionation process

| | | Profile (%)¹ | | | | | | Average | |
|---|---|---|---|---|---|---|---|---|---|
| Conditions | | 1 | 2 | 3 | 4 | 5 | 6 | (%)² | Stdev³ |
| T = 60 | BDMC | | | | | | 23.86 | | |
| P = 130 | DMC | | | | | | 14.31 | | |
| density = 0.550 | C | | | | | | 61.83 | | |
| T = 61.5 | BDMC | | 10.66 | 11.03 | 10.07 | 9.60 | 11.80 | 10.63 | 0.85 |
| P = 150 | DMC | | 14.99 | 15.40 | 17.61 | 16.56 | 15.12 | 15.93 | 1.12 |
| density = 0.600 | C | | 74.36 | 73.58 | 72.32 | 73.84 | 73.08 | 73.43 | 0.78 |
| T = 67.1 | BDMC | 9.28 | 10.22 | 9.02 | 9.98 | 8.46 | 8.72 | 9.28 | 0.70 |
| P = 150 | DMC | 13.69 | 12.74 | 12.08 | 12.95 | 13.09 | 12.77 | 12.88 | 0.53 |
| density = 0.573 | C | 77.03 | 77.05 | 78.91 | 77.07 | 78.45 | 78.51 | 77.84 | 0.88 |
| T = 67.1 | BDMC | | | | | 6.69 | 6.39 | 6.54 | 0.21 |
| P = 160 | DMC | | | | | 11.07 | 11.13 | 11.10 | 0.04 |
| density = 0.617 | C | | | | | 82.24 | 82.49 | 82.36 | 0.17 |

Notes:
¹Profile (%): calculated by: (Purity of each curcuminoid)/(Total purity of curcuminoid) × 100;
²Average: which is the arithmetic mean, and is calculated by adding a group of numbers and then dividing by the count of those numbers; and
³Stdev; The standard deviation is a measure of how widely values are dispersed from the average value (the mean). STDEV was calculated by the following formula: $\sqrt{\frac{\sum {( x - \overline{x} )}^{2}}{( n - 1 )}}$

The profile of the curcuminoids is highly depend on operation conditions, not on operation methods since the profile of curcuminoids obtained at the same conditions but different operation methods are very close with the standard deviation below 1%.

The profile of curcuminoid in feedstock is 66% Curcumin, 14% Demethoxycurcumin and 20% Bisdemethoxycurcumin, which was obtained by either exhaustive ethanol or methanol extraction. By using SCCO2 processing, the profile of curcumin (C), DMC and BDMC can be changed from 61.83%-82.36%, 11.10%-15.95%, and 6.54%-23.86%, respectively. These changes in or tuning of the relative abundances of the individual curcuminoids can not obtained by using conventional extraction methods.

### Polysaccharide Fraction

Turmeric root polysaccharides and glyco-proteins were obtained using different concentrations of ethanol for precipitations. The results are shown in Table 5.

TABLE 5
Turmeric root water leaching yield and polysaccharide purity
analysis results by using Dextran as reference standards.

| | | Purity | | | |
|---|---|---|---|---|---|
| | | calculated by | | | |
| | Yield | Dextran eq. (%) | | | Average Mw |
| Sample | (%) | 5K | 50K | 410K | (KDa) |
| Crude | 17.43 | 30.6 | 35.5 | 26.0 | |
| F20 | 2.77 | | | | 1059 |
| F40 | 5.14 | 11.2 | 10.3 | 8.2 | 1248 |
| F60 | 6.22 | 11.8 | 10.9 | 8.7 | 1132 |
| F80 | 7.90 | 26.8 | 33.2 | 23.5 | 889 |
| F95 | 10.28 | 29.0 | 33.3 | 24.5 | 788 |

Note:
F20 can not analyzed because it can not be dissolved in water to obtained a certain concentration solution.

From the data in Table 5, it can be seen that the yield of ethanol precipitated polysaccharides were in the range of 2.77-10.28%, by % mass weight based on the original turmeric feedstock. The yield was increased with increasing ethanol concentration.

From molecular weight analysis of different precipitates, it can be seen that F40 and F60 are similar; F80 and F95 are similar and F20 is different from all of them. It was also found that turmeric root polysaccharides and glycoproteins were composed of different molecular weights of polysaccharides and glycoproteins in certain ratios. In F40 and F60 polysaccharides, the highest molecular weight compound group was at 2300-2400 KDa, accounting for 46-48% by mass weight and the average molecular weight was about 1100-1200 KDa. In F80 and F95 polysaccharide-glycoprotein precipitates, the highest molecular weight components were also at 2300-2400 KDa, but accounting for only about 30-35% by mass weight. The average molecular weight was 780-890 KDa.

### Turmerin Fraction

Turmeric root protein fraction was purified by using Dowex 50-WX2-200 strong acid-cation exchange resin (—SO₃H groups as the exchange group) to process the supernatant of the 60% ethanol precipitate. The results are shown in Table 6.

TABLE 6
Protein process yield in each step and Bradford analysis results

| | | | | | | BSA eq. | BSA eq. |
|---|---|---|---|---|---|---|---|
| | Mass | Yield | C | Bradford | BSA eq. | in solution | Purity |
| Sample | (g) | (%) | (mg/ml) | abs @ 595 nm | (mg/ml) | (g) | g/g |
| F60 | 0.3 | 9.9 | 0.5 | 0.179 | 0.015 | 0.039 | 0.13 |
| supernatant | | | | | | | |
| Dowex | 0.13 | 4.5 | 0.5 | 0.564 | 0.03 | 0.04 | 0.30 |
| effluent | | | | | | | |
| Dowex elute | 0.16 | 5.4 | 0.5 | 0.547 | 0.01 | 0.01 | 0.06 |

UV spectrophotometer scanning at wavelength of 190-300 nm is used to test the wavelength at which the solution has maximum absorption. Both Dowex effluent and elute has maximum absorption at wavelength of 202 nm and loading solution has maximum absorption at wavelength of 210 nm. In addition, Dowex effluent has the highest absorption intensity, which means that there is higher concentration of polypeptide proteins in the Dowex effluent. The results in Table 6 also shows that the Dowex effluent has 0.30 g BSA eq./g extracts, which is 2.3 times higher than that in Dowex feed solution.

In general, the methods and extractions of the present invention comprise methods for making an curcuma species extraction having predetermined characteristics. Such a curcuma species extraction may comprise any one, two, three or all four of the four concentrated extract fractions depending on the beneficial biological effect(s) desired for the given product. Typically, an extraction containing all four curcuma species extraction fractions is generally desired as such novel extractions represent the first highly purified and standardized curcuma species extraction products that contain all four of the principal biologically beneficial chemical constituents found in the native plant material. Embodiments of the invention comprise methods wherein the predetermined characteristics comprise a predetermined selectively increased concentration of the curcuma species' essential oil, curcuminoids, turmerin, and polysaccharides in separate extraction fractions.

Extractions resulting from the methods of the present invention comprise extracted curcuma species plant material or a curcuma species extraction, or combination or mixture of both. Extractions comprise extracted curcuma species plant material having predetermined characteristics or an extracted curcuma species or an curcuma species extraction having a predetermined characteristic.

A further embodiment of such extractions comprises a predetermined polysaccharide concentration substantially increased in relation to that found in natural curcuma species dried plant material or conventional curcuma species extract products. For example, an extraction may comprise water soluble, ethanol insoluble polysaccharide fractions of 10% to 92% by mass weight.

Another embodiment of such extractions, a predetermined turmerin fraction concentration substantially increased in relation to that found in natural curcuma species plant material or conventional curcuma species extract products. For example, an extraction may comprise a turmerin fraction of greater than 0.2% to 6.6% by mass weight.

### Purity of Extractions

In performing the extraction methods described below, it was found that greater than 60% yield by mass weight of the curcuma species essential oil having greater than 70% purity of the three tumerones (ar-tumerone, α-tumerone, and tumerone) in the original dried rhizome feedstock of the curcuma species can be extracted in the SCCO2 essential oil extract fraction (Step 1).

Using the methods as taught in Step 1 (SCCO2 Extraction and Fractionation), a highly purified curcuminoid fraction can be extracted. The yield from this extraction step is about 22% of the curcuminoids present in the natural curcuma species feedstock. The purity (concentration) of the extracted curcuminoid extraction is greater than 80% by dry mass weight and the three principal curcuminoids have been favorably profiled (ratios altered) wherein curcumin is greater than 80% of the curcuminoids by % mass weight of the total curcuminoids. In Step 2 (ethanol leaching extraction), greater than 80% of the curcuminoids (78%) remaining in the Step 1 SCCO2 extraction and fractionation residue can be extracted. Step 3 SCCO2 purification and fractionation of the ethanol extracted curcuminoid fraction results in a highly purified (>85% curcuminoids by % dry mass weight of the extract composition) curcuminoid fraction composition with >70% curcumin by % mass weight of the curcuminoid chemical constituents in the composition. Step 4 SCCO2 purification and profiling of the curcuminoids can further purify the Step 3 curcuminoid extraction fraction to a curcuminoid fraction composition wherein the concentration of the curcuminoids is greater than 90% by mass weight with a curcuminoid profile wherein the curcumin concentration is greater than 75% of the curcuminoid chemical constituents by % mass weight. In fact, Step 4 SCCO2 purification and profiling of the curcuminoids can purify a highly concentrated curcuminoid extraction product wherein the curcuminoid concentration in the curcuminoid fraction composition is greater than 95% and the curcuminoid distribution has be profiled wherein the concentration of curcumin is greater than 85% by mass weight of the curcuminoid chemical constituents. Therefore, the SCCO2 extraction and fractionation process as taught in this invention permits the ratios (profiles) of the individual curcuminoids comprising the curcuminoid chemical constituent fraction compositions to be altered such that unique curcuminoid fraction composition profiles can be created for particular medicinal purposes.

Using the methods as taught in Steps 5 and 6 of this invention, a water soluble, ethanol insoluble extraction fraction (polysaccharide fraction composition) is achieved with a 4.5% yield from the original curcuma species feedstock having a greater than 90% purity (concentration) of curcuma polysaccharides. This further equates to a 70% yield of the curcuma species polysaccharide chemical constituents found in the natural curcuma species feedstock.

Using the methods as taught in Step 5, 6 and 7 of this invention, a turmerin fraction yield of 2.0% by % dry mass weight from the original curcuma species feedstock. The concentration of the peptides, largely turmerin, in the turmerin fraction was about 6.6% dry mass weight, a 66 fold increase in the purity of the peptides by % mass weight based on the native curcuma species feedstock. This equates to a greater than 90% yield by % mass weight of the turmerin peptide chemical constituents found in the native curcuma species plant material using the Bradford proteins analysis.

Finally, the methods as taught in the present invention permit the purification (concentration) of the essential oil fraction composition, the curcuminoid fraction composition, the polysaccharide fraction composition and the turmerin fraction composition to be as high as 70% to 90% of the desired chemical constituents in the essential oil fraction composition, as high as 97% curcuminoids in the curcuminoid fraction composition, as high as 92% polysaccharides in the polysaccharide fraction composition, and as high as 6.6% turmerin peptides in the turmerin fraction composition. The specific extraction environments, rates of extraction, solvents, and extraction technology used depend on the starting chemical constituent profile of the source material and the level of purification desired in the final extraction products. Specific methods as taught in the present invention can be readily determined by those skilled in the art using no more than routine experimentation typical for adjusting a process to account for sample variations in the attributes of starting materials that is processed to an output material that has specific attributes. For example, in a particular lot of curcuma species plant material, the initial concentrations of the essential oil, the curcuminoids, the polysaccharides, and the peptide proteins are determined using methods known to those skilled in the art as taught in the present invention. One skilled in the art can determine the amount of change from the initial concentration of the curcuminoids, for instance, to the predetermined amounts of curcuminoids for the final extraction product using the extraction methods, as disclosed herein, to reach the desired concentration in the final curcuma species composition product.

### Extractions Relative to Natural Curcuma

An embodiment of the present invention comprises a predetermined essential oil concentration wherein the predetermined essential oil concentration is a concentration of the essential oil that is greater than that which is present in the natural curcuma species plant material or conventional curcuma species extract products which can result from the extraction techniques taught herein. For example, a composition may comprise greater of 5% to 99% by mass weight of curcuma species essential oil chemical constituents. Another embodiment of the present invention comprises a predetermined curcuminoid concentration in the extracted curcuma species extraction wherein the curcuminoid concentration is greater than that found in the native plant material or conventional curcuma species extracts. For example, an extraction may comprise curcuma species curcuminoids at a concentration of 35% to 97% by mass weight. An embodiment of such curcuminoid extractions comprise a predetermined preferred purified curcuminoid distribution profile wherein the predetermined curcumin concentration is greater than that which is present in the natural curcuma species plant material or conventional curcuma species curcuminoid extraction products which can result from the extraction techniques taught herein. For example, a purified curcuminoid fraction may comprise a curcuminoid profile wherein curcumin is at a concentration of 75% to 90% by mass weight of the curcuminoids with a corresponding reduction in the concentration of demethoxycurcumin and bisdemethoxycurcumin.

Embodiments also comprise extractions wherein one or more of the fractions, including the essential oil chemical constituents, the curcuminoids, the turmerin fraction, or polysaccharides, are found in a concentration that is less than that found in native curcuma plant material. For example, extractions of the present invention comprise fractions where the concentration of the essential oil fraction is from 0.001 to 22 times the concentration of native curcuma species plant material, and/or extractions where the concentration of curcuminoids is from 0.001 to 25 times the concentration of native curcuma species plant material, and/or compositions where the concentration of the turmerin fraction is from 0.001 to 66 times the concentration of native curcuma species plant material, and/or polysaccharides is from 0.01 to 16 times the concentration of native curcuma species plant material. In making a combined extraction, from about 0.001 mg to about 100 mg of an essential oil fraction can be used; from about 0.001 mg to about 1000 mg of a curcuminoid fraction can be used; from 0.001 mg to about 100 mg of a turmerin fraction can be used; and from about 0.001 mg to about 1000 mg of the polysaccharide fraction can be used.

An embodiment of such extractions comprise predetermined concentrations of the extracted and purified and/or profiled chemical constituent fractions wherein the curcuma species essential oil/curcuminoids, essential oil/turmerin, essential oil/polysaccharides, curcuminoids/turmerin, curcuminoids/polysaccharide and turmerin/polysaccharide concentration (% dry weight) profiles (ratios) are greater or less than that found in the natural dried plant material or conventional curcuma species extraction products. Alteration of the concentration relationships (chemical profiles) of the beneficial chemical constituents of the curcuma species permits the formulation of unique or novel curcuma species extraction products designed for specific human conditions or ailments. For example, a novel and powerful curcuma extraction for anti-inflammatory activity and arthritis therapy could have a greater purified essential oil, curcuminoid (preferably having an altered curcuminoid profile wherein the concentration of curcumin is greater than 80% by mass weight of the curcuminoids) and turmerin compositions and a reduced polysaccharide composition by % mass weight than that found in the curcuma species native plant material or conventional known extraction products. In contrast, a novel curcuma extraction for immune enhancement could have a greater purified polysaccharide fraction and a reduced curcuminoid fraction and turmerin fraction by % mass weight than that found in the curcuma native plant material or conventional known extraction products. Another example of a novel curcuma extraction profile for Alzheimer's disease could be an extraction profile with greater purified essential oil and curcuminoids compositions and reduced purified turmerin and polysaccharide fractions than that found in native curcuma species native plant material or known conventional curcuma extraction products.

### Methods of Extraction

The starting material for extraction is plant material from curcuma species. C. longa L. is a preferred starting material. The material may be the aerial portion of the plant, which includes the leaves, stems, or other plant parts, though the rhizome (roots) is the preferred starting material. The curcuma species plant material may undergo pre-extraction steps to render the material into a form useful for extraction. Such pre-extraction steps include, but are not limited to, that wherein the material is chopped, minced, shredded, ground, pulverized, cut, or torn, and the starting material, prior to pre-extraction steps, is dried or fresh plant material. A preferred pre-extraction step comprises grinding and/or pulverizing the curcuma species rhizome material into a fine powder. The starting material or material after the pre-extraction steps can be dried or have moisture added to it.

### Supercritical Fluid Extraction of Curcuma

In general, methods of the present invention comprise, in part, methods wherein curcuma species plant material is extracted using novel fractionation supercritical fluid carbon dioxide (SCCO2 or SFE) extraction that is followed by one or more solvent extraction steps, such as, but not limited to, water, hydroalcoholic extractions, adsorbent resin adsorption, and additional novel fractionation SCCO2 extraction processes. Additional methods contemplated for the present invention comprise extraction of curcuma plant material using other organic solvents, refrigerant chemicals, compressible gases, sonification, pressure liquid extraction, process liquid chromatography, high speed counter current chromatography, polymer adsorbents, molecular imprinted polymers, and other known extraction methods. Such techniques are known to those skilled in the art.

The invention includes a process for extracting the oleoresin from turmeric plant material using SCCO2. The invention includes the fractionation of the oleoresin extracts into, for example, the essential oil and the curcuminoid chemical components with high purity. Moreover, the invention includes a SCCO2 process wherein the individual chemical constituents within an extraction fraction may have their chemical constituent ratios or profiles altered. For example, SCCO2 fractional separation of the curcuminoids permits the selective extraction of curcumin relative to the other curcuminoids such that a curcuminoid extract fraction can be produced with a concentration of curcumin greater than 80% of the curcuminoids present in the purified curcuminoid extract fraction.

“Fractional extraction” and “fractional separation” of plant oleoresins using SCCO2 (See U.S. Pat. No. 5,120,558) enables the selective extraction of the curcuma essential oil chemical constituents under relatively mild conditions (temperatures of 50° C. or less, pressures of 300 bar or less). Subsequently, it is then possible to re-extract the curcuma feedstock material under more severe conditions (temperatures >50° C., pressures >300 bar) to obtain curcuminoid chemical constituents, which are generally less soluble in SCCO2 fluid. As a result, two highly purified fractions are obtained: the light fraction (essential oil fraction) and the heavy fraction (curcuminoid fraction). Additional fractionation of the extract fractions at high temperatures and pressures takes place simultaneously by passing the extract/fluid stream through a series of 3 separators. Pressure and temperature conditions in each separator vessel are precisely chosen to precipitate an individual chemical constituent of interest, such as, but not limited to, curcumin.

The supercritical fluid extraction and fractionation system is a material processing system designed for the production of medicinal products from botanical sources using SCCO2. The system is equipped with features that enable suitable preprocessed natural botanical feedstock material to be loaded within a processing vessel, exposed to a pressurized CO2 stream to remove selected chemical constituent, and subsequently passed through chemical process equipment (separators) that selectively separate the desired chemical constituents from the main CO2 stream.

The SCCO2 system is comprised of two main extraction vessels, three separation vessels, electrical heat exchangers, fluid-cooled condensers, CO2 accumulator, mass flow meters, CO2 pump, additive pump and chiller. The primary extraction vessels are 24 L, fabricated from 17-4PH stainless steel and pressure rated to 700 bar (11,000 psi). The separation vessels are 20 L, fabricated with 316 stainless steel and pressure rated to 200 bar (3000) psi. Each extractor and separator is equipped with a quick-acting closure system, which enables a short loading and unloading time of the extraction system.

All pressure-bearing parts are protected against over pressure by safety valves. Various interlocks are integrated into the system to prevent operation failures. In case of failure of the instrument or energy source, all pneumatic actuated valves will go to a failsafe mode. An additive pump is used to dose co-solvents such as ethanol into the CO2 at a flow rate of 0.5 L/min. To prevent the pump from cavitating, liquid CO2 flows from the CO2 storage vessel through a cooler to the CO2 pump. The CO2 is compressed to the desired extraction pressure using the CO2 pump and heated to the extraction temperature with a heater. The system is rigorously controlled using two National Instruments compact fieldpoint processors (CFP-2020 and CFP-200). National Instrument Labview RT (real time) runs on these processors using a custom software application. The CPF are interfaced via Ethernet to the operator interface computers.

In brief, the process comprises liquefied CO2 flowing from the CO2 storage vessel through a cooler to the CO2 pump. Then the CO2 is compressed to the desired extraction pressure and heated to the desired temperature. The extractor vessels are filled with baskets of pretreated botanical feedstock material and operated alternatively or in series. During the operation of the system, one extractor vessel is in the CO2 circuit while the other one could be depressurized, the feedstock exchanged, and this extractor vessel re-pressurized. This latter mode of operation leads to a semi-continuous solid material flow. Separation is carried out in three rigorously controlled steps, high pressure, medium pressure, and low pressure with appropriate temperature adjustment for each separator. The CO2 after passage through the separators is now free of extract and flows to a condenser, where it is liquefied. The liquid CO2 then flows into the CO2 storage vessel for recycling.

Extraction of the oleoresin of curcuma species with SCCO2 as taught in the present invention eliminates the use of organic solvents and provides simultaneous fractionation of the extracts. Carbon dioxide is a natural and safe biological product and an ingredient in many foods and beverages. Unlike conventional SCCO2 which is capital intensive, operates in a discrete batch mode, not cost-effective compared to solvent extraction methods. In the present invention, the SCCO2 fractional extraction and separation system overcomes these limitations

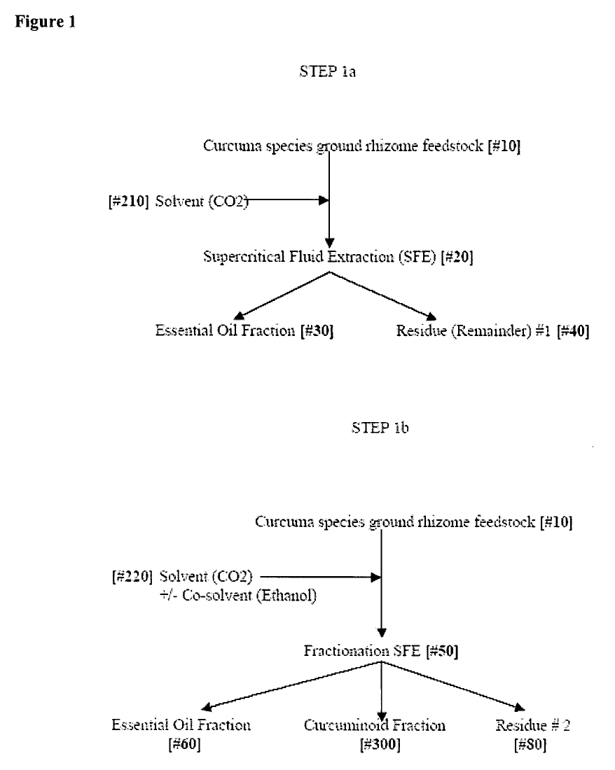

A schematic diagram of the methods of extraction of the biologically active chemical constituents of curcuma species is illustrated in FIGS. 1-7. The extraction process is typically, but not limited to, 7 steps. For reference in the text, when the symbol # appears in brackets [#x], the following number refers to the numbers in FIGS. 1-7. The analytical methods used in the extraction process are presented in the Exemplification section.

### Step 1. Essential Oil Extraction Processes

Due to the hydrophobic nature of the curcuma species essential oil, non-polar solvents, including, but not limited to supercritical fluid extraction (SFE) such as SCCO2, hexane, petroleum ether, and ethyl acetate as well as steam distillation may be used for this extraction process.

This process method comprises a single extraction step for purifying (concentrating) the essential oil (FIG. 1a) or, if desired, purifying the essential while simultaneously purifying the curcuminoids and altering the ratios of the individual curcuminoid compounds within the curcuminoid chemical group (FIG. 1b).

A generalized description of the supercritical fluid extraction (SFE) fractionation extraction of the essential oil fraction from the native curcuma species feedstock is diagrammed (FIG. 1-Step 1). The feedstock [#10] is dried, ground curcuma species rhizome feedstock (8-20 mesh). The feedstock is loaded into a basket that is placed inside a SFE extraction vessel [#20 or #50]. The solvent [#210 or #220] is pure carbon dioxide (CO2). 95% ethanol may be used as a co-solvent [#220]. After purge and leak testing, the process comprises liquefied CO2 flowing from a storage vessel through a cooler to the CO2 pump. The CO2 is compressed to the desired pressure and then flows through the feedstock in the extraction vessel where the pressure and temperature are maintained at the desired level. The pressures for extraction range from about 100 bar to 800 bar, from about 200 bar to about 600 bar, from about 300 to about 400 bar, and the temperature ranges from about 30° C. to about 100° C., and from about 40° C. to about 90° C., and from about 60° C. to about 80° C. The SCCO2 extractions taught herein are preferably performed at pressures of at least 100 bar and a temperature of at least 30° C., and more preferably at a pressure of about 300 to about 600 bar and at a temperature of about 50° C. to 90° C. The time for extraction range from about 30 minutes to about 2.5 hours, from about 1 hour to about 2 hours, to about 1.5 hours. The solvent to feed ratio is typically 50 to 1 for each of the SCCO2 extractions. The CO2 is recycled. The extracted and purified essential oil and the extracted, purified, and profiled curcuminoid fraction(s) is then collected in a collector or separator vessel [#30 & #40 or #60, #300, & #80], saved and stored in the dark at 4° C. The curcuma species ground rhizome feedstock material [#10] may be extracted in a one-step process wherein the resulting extracted curcuma essential oil fraction is collected in a one collector SFE or SCCO2 [#20] system (Step 1, above). Alternatively, as in a fractional SFE system [#50] the SCCO2 extracted curcuma species feedstock material may be segregated into collector vessels (separators) [#60, #300, #80] such that within one of the collector (separator) vessels there is a purified essential oil fraction [#60], in second collector vessel there is purified and profiled curcuminoid fraction [#300] and in a third collector vessel there is the residue or remainder [#80] of the extracted curcuma species rhizome material. An embodiment of the invention comprises extracting the curcuma species natural rhizome material using fractional SCCO2 extraction at 300 to 600 bar and at a temperature between 50° C. and 90° C. and collecting the extracted curcuma species material in differing collector vessels at predetermined conditions (pressure, temperature, and density) and predetermined intervals (time).

The resulting extracted curcuma species purified essential oil fraction can be retrieved and used independently or can be combined to form one or more extracted curcuma species extractions. An aspect of the SCCO2 extracted essential oil fraction comprises a predetermined essential oil chemical constituent concentration that is higher than that found in the native plant material or in conventional curcuma species extraction products. Typically, the total yield of essential oil chemical constituents is greater than 95% and the purity of the essential oil chemical constituents in the essential oil extracted fraction is greater than 99% by mass weight. The purity and chemical constituents in the essential oil fraction may be measured using Gas Chromatography-Mass Spectroscopy (GC-MS) analysis. Analytical results from such extractions are shown in Tables 7 and 8. Experimental examples of such extractions are found below. The resulting extracted curcuma species purified and profiled curcuminoid fraction can be retrieved independently and used independently or can be combined to form one or more curcuma species extractions.

An aspect of the SCCO2 extracted curcuminoid fraction comprises a predetermined curcuminoid chemical constituent concentration combined with curcuminoid concentration profile wherein curcuma is higher than that found in the native plant material or in conventional curcuma extraction products. Typically, the total yield of curcuminoid fractions from curcuma species feedstock is about 22% of the curcuminoids by % mass weight, having a curcuminoid fraction purity of greater than 80% and a curcuminoid fraction profiled chemical constituent of greater than 80% curcuma by % mass weight of the curcuminoids. The purity and curcuminoid distributions are measured using HPLC analysis. Examples as well as the results of such extraction processes are found in Example 1 and in Tables 9 and 10.

### Step 2. Ethanol Leaching Extraction

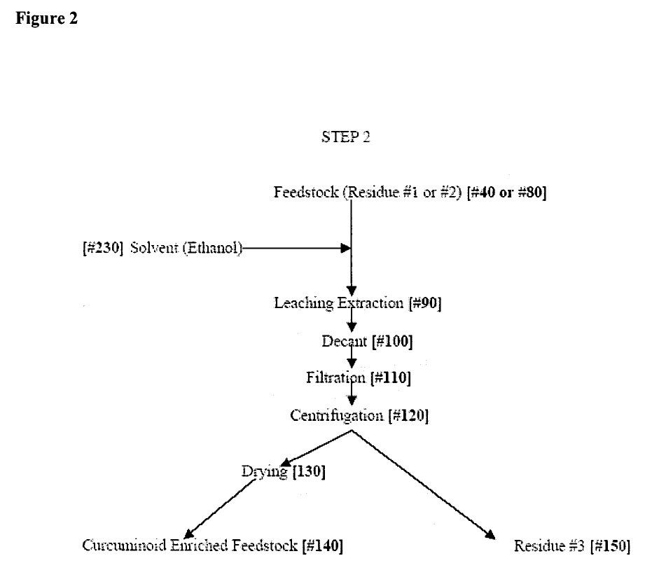

A generalized description of the extraction of curcuma species residue material [#40 or #80] from the Step 1 SCCO2 extraction process using an ethanol leaching process is diagrammed in FIG. 2-Step 2. The feedstock [#40 or #80] is the residue from either Step 1a or Step 1b. The extraction solvent [#230] is 95% ethanol. In this method, the feedstock and the extraction solvent are separately loaded into an extraction vessel heated to 60 to 80° C. and stirred for 3 to 7 hours. After the mixing is discontinued, the solution is allowed to stand for 10 to 20 hours. The top layer was decanted [#100], filtered [#110], centrifuged [#120]. The curcuminoid enriched supernatant was evaporated [#130] to a tart or powder [#140]. This dried extraction product [#140] is then used for further processing (Step 3). The solid residue [#150] may be saved and used for further processing (Step 4) to obtained purified fractions of curcuma species polysaccharides and polypeptides (turmerin). An example as well as the results of these extraction processes is found in Example 3 and in Table 11.

### Step 3. SCCO2 Purification of the Ethanol Extracted Curcuminoid Fraction

This process method comprises a single extraction step for purifying (concentrating) the curcuminoids and, if desired, altering the ratios of the individual curcuminoids within the curcuminoid chemical group. In a preprocessing step, the essential oil in the natural curcuma species feedstock is extracted using SCCO2 (Step 1) and the curcuminoids are then extracted from the residue of Step 1 using ethanol (Step 2) and either vacuum dried to form a tart form as taught in Step 2 and mixed with glass beads to form a flowable powder or spray dried to a powder form (particle size greater than 100 μm).

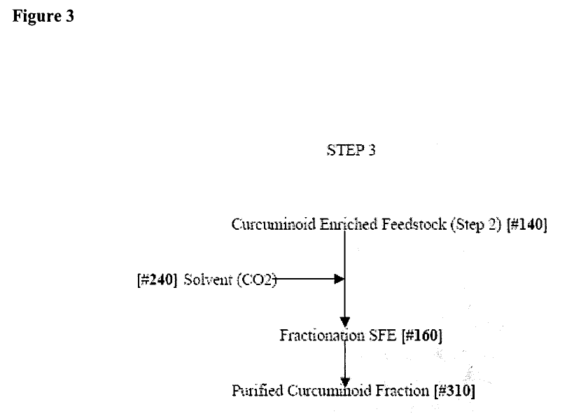

A generalized description of the SFE fractionation extraction of the curcuminoid fraction from the extraction product of Step 2 [#140] is diagrammed in FIG. 3-Step 3. The feedstock [#140] is mixed with glass beads and loaded into an SFE extraction vessel [#160]. The solvent is pure carbon dioxide [#240]. Ethanol may be used as a co-solvent. After purge and leak testing, the process comprises liquefied CO2 flowing from a storage vessel through a cooler to the CO2 pump. The CO2 is compressed to the desired pressure and then flows through the feedstock in the extraction vessel where the pressure and temperature are maintained at the desired level. The pressures for extraction range from about 100 bar to 800 bar, from about 200 bar to 700 bar, from about 300 bar to 600 bar and the temperature ranges from about 30° C. to about 100° C., from about 45° C. to about 95° C., and from about 60° C. to about 90° C. The SCCO2 extractions taught herein are preferably performed at pressures of at least 300 bar and a temperature of at least 40° C., and more preferably at a pressure of about 400 bar to about 600 bar and at a temperature of about 60° C. to about 90° C. The time of extraction ranges from about 30 minutes to 4 hours, from about 1 hour to 3 hours, to about 2 hours. The solvent to feed ratio is typically about 1000 to 1 for each of the SCCO2 extractions. The CO2 is recycled. The extracted, purified and profiled curcuminoid fractions are then collected in collector or separator vessels [#310] that have predetermined set pressures and temperatures.

An embodiment of the invention comprising extracted either the ethanol enriched curcuminoid material or an extracted enriched curcuminoid material using fractional SCCO2 extraction at 300 bar to 600 bar and at a temperature between 60° C. and 95° C. and collecting the extracted curcuminoid fraction material in differing collector vessels at predetermined conditions (pressure, temperature, and density) and predetermined intervals (time). The resulting extracted curcuma species purified curcuminoid fraction in each collector can be retrieved or used independently or can be combined to form one or more curcuma species extraction products. An aspect of the SCCO2 extracted curcuma species curcuminoid fraction comprises a predetermined curcuminoid chemical constituent concentration that is higher than that found in the native curcuma species plant material or in conventional curcuma species extraction products. A further aspect of the invention is a purified extracted curcuminoid fraction wherein the concentration of the curcuma is greater than 70% mass weight of the curcuminoid chemical constituents mass weight. Typically, the total yield of the purified curcuminoid fraction from the curcuma species native rhizome material is about 2.6% having a curcuminoid concentration of greater than 85% curcuminoids by mass weight of the curcuminoid extraction fraction. Moreover, the concentration profile of the curcuminoids can be altered to a curcuma concentration of greater than 70% by mass weight. An example and the results of such extraction processes are found in Example 4 and in Table 12.

### Step 4. Purification and Profiling of the Curcuminoids

This process method comprises a single extraction step for additional purification (concentrating) of the curcuminoids and, if desired, altering the ratios of the individual curcuminoids within the curcuminoid chemical group. In a preprocessing step, the essential oil in the natural curcuma species feedstock is extracted using SCCO2 (Step 3) and either vacuum dried to form a tart form and mixed with glass beads to form a flowable powder or spray dried to a powder form (particle size greater than 100 μm). In another preprocessing step, a highly enriched curcuminoid extraction product is mixed with glass beads to form a flowable powder.

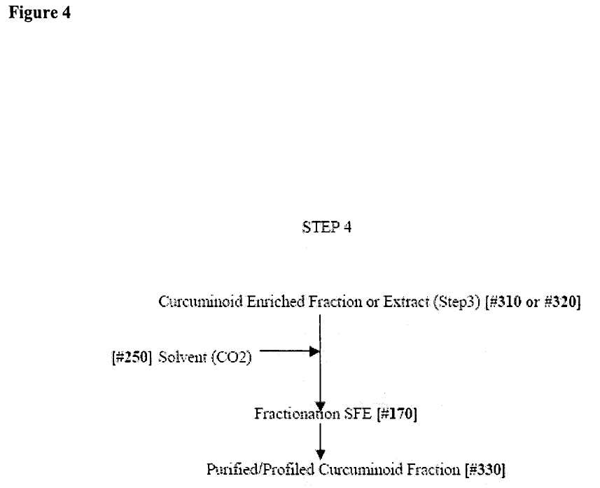

A generalized description of the SFE fractionation extraction of the curcuminoid fraction from the extraction product of Step 3 [#310] or a highly enriched curcuminoid extraction product [#320] is diagrammed in FIG. 4-Step 4. The feedstock [#310 or #320] is mixed with glass beads and loaded into an SFE extraction vessel [#170]. The solvent is pure carbon dioxide [#250]. Ethanol may be used as a co-solvent. After purge and leak testing, the process comprises liquefied CO2 flowing from a storage vessel through a cooler to the CO2 pump. The CO2 is compressed to the desired pressure and then flows through the feedstock in the extraction vessel where the pressure and temperature are maintained at the desired level. The pressures for extraction range from about 100 bar to 800 bar, from about 200 bar to 700 bar, from about 300 bar to 600 bar and the temperature ranges from about 30° C. to about 100° C., from about 45° C. to about 95° C., and from about 60° C. to about 90° C. The SCCO2 extractions taught herein are preferably performed at pressures of at least 300 bar and a temperature of at least 40° C., and more preferably at a pressure of about 400 bar to about 600 bar and at a temperature of about 60° C. to about 90° C. The time of extraction ranges from about 30 minutes to 4 hours, from about 1 hour to 3 hours, to about 2 hours. The solvent to feed ratio is typically about 1000 to 1 for each of the SCCO2 extractions. The CO2 is recycled. The extracted, purified and profiled curcuminoid fractions are then collected in collector or separator vessels [#330] that have predetermined set pressures and temperatures. An embodiment of the invention comprising extracted either the ethanol enriched curcuminoid material or an extracted enriched curcuminoid material using fractional SCCO2 extraction at 300 bar to 600 bar and at a temperature between 60° C. and 95° C. and collecting the extracted curcuminoid fraction material in differing collector vessels at predetermined conditions (pressure, temperature, and density) and predetermined intervals (time). The resulting extracted curcuma species purified curcuminoid fraction in each collector can be retrieved or used independently or can be combined to form one or more curcuma species extraction products.

An aspect of the SCCO2 extracted curcuma species curcuminoid fraction comprises a predetermined curcuminoid chemical constituent concentration that is higher than that found in the native curcuma species plant material or in conventional curcuma species extraction products.

A further aspect of the invention is a purified extracted curcuminoid fraction wherein the concentration of the curcuma is greater than 80% of the curcuminoid chemical constituents by % mass weight. Typically, the total yield of the purified curcuminoid fraction from the curcuma species native rhizome material is about 0.9% mass weight having a curcuminoid concentration of greater than 85% curcuminoids by mass weight. Moreover, the concentration profile of the curcuminoids can be altered to a curcuma concentration of greater than 75% mass weight of the curcuminoids. With respect to the highly enriched curcuminoid extraction product, the yield is greater than 60% by mass weight with a curcuminoid purity of greater than 95% and a curcuminoid profile wherein curcuma is greater than 85% of the curcuminoids by % mass weight. Examples and the results of such extraction processes are found in Example 5 and in Tables 13, 14 & 15.

### Step 5. Water Leaching of Residue of Step 2

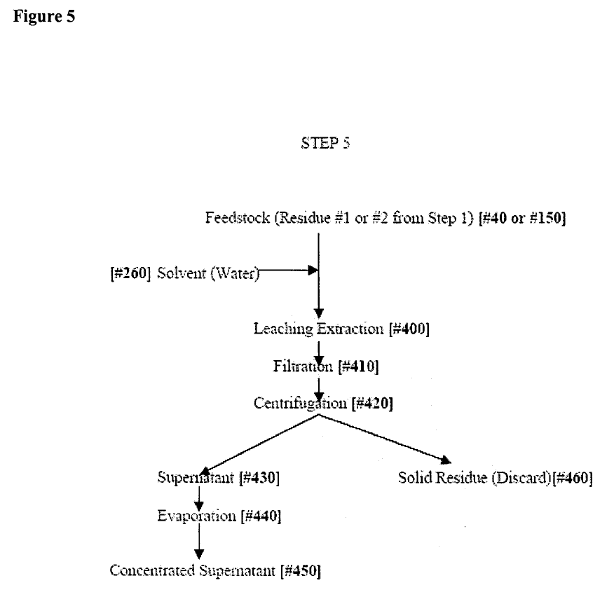

In one aspect, the present invention comprises extraction and concentration of the bio-active polysaccharide and polypeptide (tumerin) chemical constituents of curcuma species plant material. A generalized description of a preparatory extraction step is diagrammed in FIG. 5-Step 5. This Step 5 extraction process is a single stage solvent leaching process. The feedstock for this extraction process is the residue of Step 1b [#40] or Step 2 [#150]. The extraction solvent [#260] is distilled water. In this method, the curcuma species residue and the extraction solvent are loaded into an extraction vessel [#400] and heated and stirred. It may be heated to 100° C., to about 90° C. or to about 70-90° C. The extraction is carried out for about 1 to 5 hours, for about 2-4 hours, or for about 3 hours. The resultant fluid extraction is filtered [#410] and centrifuged [#420]. The supernatant [#430] was evaporated [#440] to a concentrated supernatant [#450] for further processing (Steps 6 & &). The solid residue is discarded [#460]. An example of this extraction step is found in Example 6 and the results in Table 16.

### Step 6. Polysaccharide Fraction Extraction and Purification

As taught herein, a purified polysaccharide fraction extract from the curcuma species may be obtained by ethanol precipitation of the water soluble, ethanol insoluble polysaccharides from an aqueous extract of curcuma species feedstock and then contacting the precipitate in aqueous solution with a solid polymer resin adsorbent so as to adsorb the smaller molecules of molecular weight of less than 700 D contained in the aqueous solution. The polysaccharides are then concentrated in the effluent. The bound molecules are eluted and discarded. Prior to separation of the chemical constituents in the aqueous precipitate solution, the molecular size adsorbent with the undesired chemical constituents adsorbed thereon may be separated from the effluent (desired chemical constituents) in any convenient manner, preferably, the process of contacting the adsorbent and the separation is effected by passing the aqueous extraction product through an extraction column or bed of the adsorbent material.

A variety of adsorbents can be utilized to purify the polysaccharide chemical constituents of curcuma species. A molecular size separation adsorbent such as Sephadex G-10 is preferably used to separate molecules less than 700 molecular weight from the larger molecular weight polysaccharide molecules.

Preferably, the curcuma species native feedstock material has undergone a one or more preliminary purification process such as, but not limited to, the processes described in Step 1, 2, and 5 prior to contacting the aqueous polysaccharide chemical constituent containing extract with the affinity adsorbent.

Using affinity adsorbents as taught in the present invention results in highly purified polysaccharide chemical constituents of curcuma species that are remarkably of other chemical constituents which are normally present in natural plant material or in available commercial extraction products. For example, the processes taught in the present invention can result in purified polysaccharide extracts that contain total polysaccharide chemical constituents in excess of 90% by dry mass weight.

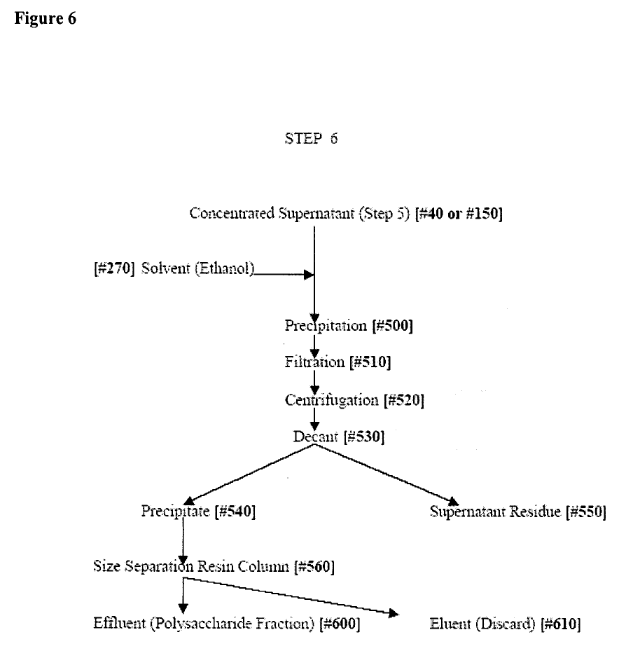

A generalized description of the extraction and purification of the polysaccharides from the rhizome of the curcuma species using ethanol precipitation and affinity adsorbent resin beads is diagrammed in FIG. 6-Step 6. The feedstock [#450] for this extraction may be the concentrated water extract solution containing the polysaccharides from Step 5 Water Leaching Extraction. The solvent [#270] used for precipitation of the polysaccharides from the aqueous solution is ethanol. The concentrated supernatant solution [#450] is diluted adding sufficient ethanol [#270] to yield a maximal precipitation [#500] of the water soluble, ethanol insoluble polysaccharides. The solution is filtered [#510], centrifuged [#520] and decanted [#530]. The supernatant residue [#550] is collected and saved for further processing to extract and purify the turmerin fraction chemical constituents of curcuma species. The precipitate [#540] is collected and the ethanol and water in the precipitate is removed by evaporation. The appropriate amount of adsorbent resin beads [#560] are cleaned and hydrated to make a slurry and loaded onto a column. The polysaccharide precipitate extract is dissolved in water to make a 1% solution and loaded onto the column [#560]. The effluent [#600] is collected, analyzed for polysaccharides, dried and saved as polysaccharide product. An example of this extraction process is found in Example 7.

### Step 7. Turmerin Fraction Extraction and Purification

As taught herein, a purified turmerin polypeptide fraction extract from curcuma species may be obtained by diluting the aqueous ethanol solution supernatant residue extract of Step 6 with a phosphate buffered saline solution and contacting this diluted extract solution with a solid size separation affinity adsorbent followed by collection of the effluent and contacting the effluent with a cation exchange resin column so as to remove impurities of lower molecular weight than turmerin and impurities that ion exchange with the cation exchange resin column, respectively. The effluent is collected and saved as product by methods taught herein. The bound chemicals (impurities) are subsequently eluted from each of the adsorbents leading to regeneration of the ion exchange resin.

Although a variety of adsorbents can be used to purify the turmerin chemical constituent fraction, preferably Sephadex G-10 is used as the size separation adsorbent to adsorb impurities of 700 molecular weight or less (molecular weight of turmerin is 5,000) and Dowex 50-WXZ-200, a strong acid cation exchange resin beads having sulfonic acid exchange groups, is used as the cation exchange adsorbent.

Preferably, the curcuma species feedstock has undergone one or more preliminary purification processes such as, but not limited to, the processes described in Step 1, 2, 5, and 6 prior to contacting the aqueous turmerin containing extract with the affinity adsorbent resin beads.

Using affinity adsorbents as taught in the present invention results in significant purification of turmerin from curcuma species plant material compared the turmerin concentration normally present in natural plant material or in available commercial extraction products. For example, the processes taught in the present invention can result in an increase in the concentration of turmerin from about 0.1% by mass weight in the natural curcuma species rhizome to about 6.6% by mass weight in the final turmerin fraction extraction product, a 66 fold increase in the concentration over that found typically in the natural curcuma species feedstock.

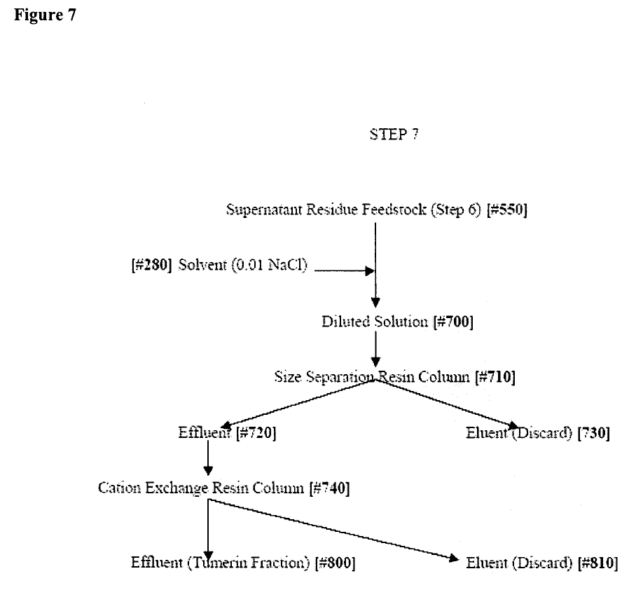

A generalized description of the extraction and purification of the turmerin fraction from extracts of the rhizome of curcuma species using affinity adsorbent resin beads is diagrammed in FIG. 7-Step 7.

The feedstock [#550] for the first extraction process may be the aqueous solution residue containing the polypeptide turmerin from Step 6 Polysaccharide Purification. The solvent [#280] used to dilute the feedstock solution is phosphate buffered saline solution to a final concentration of 1 mg/ml. The diluted feedstock solution [#700] is loaded into a column packed with a bed of clean and hydrated slurry of Sephadex G-10 beads [#710] at a flow rate of about 0.5 bed volume/hour. The effluent [#720] was collected and saved for further processing. The resin beads were eluted, cleaned and recycled. The eluent [#730] was discarded.

The feedstock [#720] for the second extraction process may be the effluent solution from the first extraction process using the size separation resin column. The feedstock solution is loaded into a column packed with a bed of clean 0.1M HCl soaked Dowex 50-WX2-200 resin bead slurry [#740]. Prior to loading the feedstock solution, the column was washed with 3 bed volumes of distilled water. The feedstock loading flow rate is about 3.4 bed volume/hour. The effluent [#800] was collected, analyzed for peptide protein content, dried and saved as the final turmerin fraction product. The Dowex resin beads were eluted, cleaned and recycled. The eluent was discarded. An example of this extraction process is found in Example 8 and the results in Table 18.

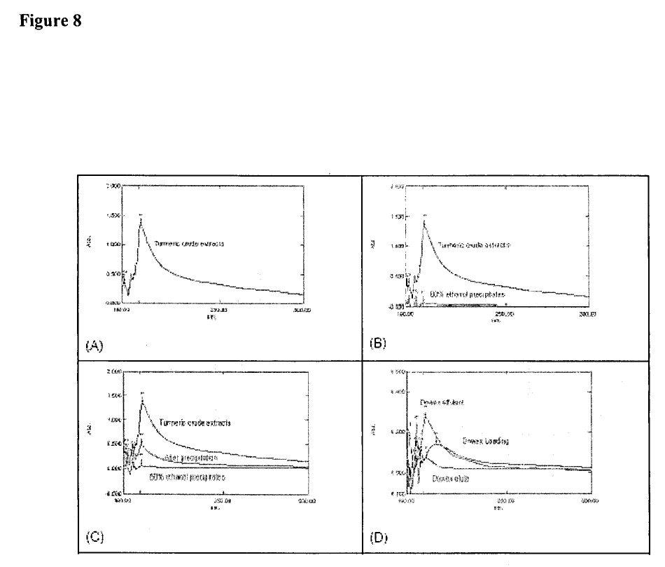

Bradford protein analysis was used to calculate the total protein in each sample. In the crude water extract, there was 26.4% ([0.82/3.1]×100=24.4%) protein content of the dissolved mass which was a 2.73% total protein yield by mass weight based on the original feedstock. As illustrated in FIG. 8A an absorbance peak at 202 nm consistent with the peptide turmerin was observed. In contrast, although the protein content in the 60% ethanol precipitate was 2.2%, no absorbance peak at around 202 nm was observed (FIGS. 8B & C). The remaining solution after 60% ethanol precipitation, there was 10% protein in the solution with a 202 nm absorbance peak (FIG. 8C). After Sephadex column removal of impurities of less than 700 MW (turmerin has a MW of 5,000), there was 3.7% protein by mass weight in the effluent solution with preservation of the 202 nm absorbance peak (FIG. 8D)—Dowex loading solution). The loading solution for the Dowex column was the Sephadex effluent dissolved in pH 7.4 phosphate buffered saline solution pH 7.4. The isoelectric point of turmerin is 4.2 so that it will be positively charged if the pH of the solution is less than its isoelectric point. Hence, the turmerin will be negatively charged in the loading solution and will not bind with the Dowex cation exchange column. Therefore, the turmerin will be in the Dowex column effluent which is confirmed by the high 202 nm absorbance (due peptic bonds) found in the effluent solution (FIG. 8D). The Dowex effluent turmerin fraction product has a 0.04 gm bovine serum albumin (BSA) equivalent and a total yield of 0.12% by mass weight based on the original curcuma species feedstock. In the Dowex column effluent or turmerin fraction, the protein content was 6.6% which indicates that the concentration of turmerin peptide is increased from about 0.1% concentration of the original raw feedstock material to 6.6% concentration by dry mass weight, a 66 fold increase in concentration over that found in the natural curcuma species feedstock.

### Food and Medicaments

As a form of foods of the present invention, there may be formulated to any optional forms, for example, a granule state, a grain state, a paste state, a gel state, a solid state, or a liquid state. In these forms, various kinds of substances conventionally known for those skilled in the art which have been allowed to add to foods, for example, a binder, a disintegrant, a thickener, a dispersant, a reabsorption promoting agent, a tasting agent, a buffer, a surfactant, a dissolution aid, a preservative, an emulsifier, an isotonicity agent, a stabilizer or a pH controller, etc. may be optionally contained. An amount of the curcuma extract to be added to foods is not specifically limited, and for example, it may be about 10 mg to 5 g, preferably 50 mg to 2 g per day as an amount of take-in by an adult weighing about 60 kg.

In particular, when it is utilized as foods for preservation of health, functional foods, etc., it is preferred to contain the effective ingredient of the present invention in such an amount that the predetermined effects of the present invention are shown sufficiently.

The medicaments of the present invention can be optionally prepared according to the conventionally known methods, for example, as a solid agent such as a tablet, a granule, powder, a capsule, etc., or as a liquid agent such as an injection, etc. To these medicaments, there may be formulated any materials generally used, for example, such as a binder, a disintegrant, a thickener, a dispersant, a reabsorption promoting agent, a tasting agent, a buffer, a surfactant, a dissolution aid, a preservative, an emulsifier, an isotonicity agent, a stabilizer or a pH controller.

An administration amount of the effective ingredient (curcuma extract) in the medicaments may vary depending on a kind, an agent form, an age, a body weight or a symptom to be applied of a patient, and the like, for example, when it is administrated orally, it is administered one or several times per day for an adult weighing about 60 kg, and administered in an amount of about 10 mg to 5 g, preferably about 50 mg to 2 g per day. The effective ingredient may be one or several components of the curcuma extract.

### Delivery Systems

Administration modes useful for the delivery of the extractions of the present invention to a subject include administration modes commonly known to one of ordinary skill in the art, such as, for example, powders, sprays, ointments, pastes, creams, lotions, gels, solutions, patches and inhalants.

In one embodiment, the administration mode is an inhalant which may include timed-release or controlled release inhalant forms, such as, for example, liposomal formulations. Such a delivery system would be useful for treating a subject for SARS, bird flu, and the like. In this embodiment, the formulations of the present invention may be used in any dosage dispensing device adapted for intranasal administration. The device should be constructed with a view to ascertaining optimum metering accuracy and compatibility of its constructive elements, such as container, valve and actuator with the nasal formulation and could be based on a mechanical pump system, e.g., that of a metered-dose nebulizer, dry powder inhaler, soft mist inhaler, or a nebulizer. Due to the large administered dose, preferred devices include jet nebulizers (e.g., PARI LC Star, AKITA), soft mist inhalers (e.g., PARI e-Flow), and capsule-based dry powder inhalers (e.g., PH&T Turbospin). Suitable propellants may be selected among such gases as fluorocarbons, hydrocarbons, nitrogen and dinitrogen oxide or mixtures thereof.

The inhalation delivery device can be a nebulizer or a metered dose inhaler (MDI), or any other suitable inhalation delivery device known to one of ordinary skill in the art. The device can contain and be used to deliver a single dose of the formulations or the device can contain and be used to deliver multi-doses of the extractions of the present invention.

A nebulizer type inhalation delivery device can contain the extractions of the present invention as a solution, usually aqueous, or a suspension. In generating the nebulized spray of the extractions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device may comprise a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer may be employed to ensure the sizing of particles is optimal for positioning of the particle within, for example, the pulmonary membrane.

A metered dose inhalator (MDI) may be employed as the inhalation delivery device for the extractions of the present invention. This device is pressurized (pMDI) and its basic structure comprises a metering valve, an actuator and a container. A propellant is used to discharge the formulation from the device. The extraction may consist of particles of a defined size suspended in the pressurized propellant(s) liquid, or the extraction can be in a solution or suspension of pressurized liquid propellant(s). The propellants used are primarily atmospheric friendly hydrofluorocarbons (HFCs) such as 134a and 227. Traditional chlorofluorocarbons like CFC-11, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid-containing formulation. To insure accuracy of dosing, the delivery of the formulation may be programmed via a microprocessor to occur at a certain point in the inhalation cycle. The MDI may be portable and hand held.

In another embodiment, the delivery system may be a transdermal delivery system, such as, for example, a hydrogel, cream, lotion, ointment, or patch. A patch in particular may be used when a timed delivery of weeks or even months is desired.

In another embodiment, parenteral routes of administration may be used. Parenteral routes involve injections into various compartments of the body. Parenteral routes include intravenous (iv), i.e. administration directly into the vascular system through a vein; intra-arterial (ia), i.e. administration directly into the vascular system through an artery; intraperitoneal (ip), i.e. administration into the abdominal cavity; subcutaneous (sc), i.e. administration under the skin; intramuscular (im), i.e. administration into a muscle; and intradermal (id), i.e. administration between layers of skin. The parenteral route is sometimes preferred over oral ones when part of the formulation administered would partially or totally degrade in the gastrointestinal tract. Similarly, where there is need for rapid response in emergency cases, parenteral administration is usually preferred over oral.

### Methods of Treatment

Methods of the present invention comprise providing novel curcuma extractions for the treatment and prevention of human disorders. For example, a novel curcuma species extraction for treatment of allergies, arthritis, rheumatism, cardiovascular disease, hypercholesterolemia, platelet aggregation, cerebrovascular disease, asthma, chronic pulmonary disease, cystic fibrosis, wound healing, Alzheimer's and Parkinson's disease, multiple sclerosis, peptic ulcer disease, cancer, HIV/AIDS, bacterial, and fungal infections may have an increased essential oil fraction concentration, an increased curcuma fraction concentration, and an increased polysaccharide fraction concentration by weight % than found in the curcuma species native plant material or conventionally known products.

A preferred method of treatment includes methods of treating arthritis comprising administering to a subject in need thereof a therapeutically effective amount of a curcuma extraction of the present invention. In a particularly preferred embodiment, the curcuma extraction further comprises a synergistic amount of similarly obtained extracts of Boswellia species, in particular the Boswellia components α- and/or β-boswellic acid and/or their C-acetates. Methods of extracting Boswellia species are fully described in the provisional patent application filed by the inventors on Sep. 21, 2006, and is hereby incorporated in its entirety. The synergism refers to the increased effect extracts of curcuma and boswellia combined have on arthritis compared to the effect each extract has individually.

The foregoing description includes the best presently contemplated mode of carrying out the present invention. This description is made for the purpose of illustrating the general principles of the inventions and should not be taken in a limiting sense. This invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

All terms used herein are considered to be interpreted in their normally accepted usage by those skilled in the art. Patent and patent applications or references cited herein are all incorporated by reference in their entireties.

### EXEMPLIFICATION

### Materials and Methods

Curcuma Feedstock

Two ground turmeric root was from different sources have been used for currect study. Turmeric extract (Lot #: CL/02005) was purchased from Suan Farma Inc. Activate component analysis results are shown in Table 7.

TABLE 7
Feedstock information for turmeric root and extract used in this study

| | Turmeric | |
|---|---|---|
| Analyte | root | Turmeric extract² |
| Vendor | Hara spices | Suan Farma |
| Essential oil | 6.64¹ | N/A |
| Total curcuminoid (wt %) | 6.82 | 91.14 |
| Curcumin (wt %) | 4.56 | 68.22 |
| DMC (%) | 1.36 | 19.63 |
| BDMC (%) | 0.90 | 4.81 |
| Polysaccharide (%) | 5.9 | N/A |
| Protein | 2.78 | N/A |

Note:
¹essential oil concentration was measured by hexane exhausted extraction for 14 hours. The total yield as 7.24% and the curcuminoid was 0.6%, so the essential oil was 7.24 − 0.6 = 5.96%.
²Information was provided by vendor dated at September 2000.

Reference Standards and Organic Solvents

Curcuminoid standards was purchased from ChromaDex, Inc. 2952 S. Daimler St. Santa Ana Calif. 92705 Tel: 949, 419, 0288, Fax: 949, 419, 0294 www.chromadex.com, and their properties is listed in Table 8.

TABLE 8
Physical properties of curcuminoid standard.

| | | | | | Chemical |
|---|---|---|---|---|---|
| Part/Lot No. | Product name | Formula | Tm Mw | (° C.) | family |
| 03924-724 | Curcumin (458-37-7) | C₂₁H₂₀O₆ | 368.4 | 183 | Phenolic acids |
| 04230-727 | Demethoxycurcumin | C₂₀H₁₈O₅ | 338.4 | N/A | Phenolic acids |
| | (24393-17-1) | | | | |
| 04231-531 | Bis-Demethoxycurcumin | C₁₉H₁₆O₄ | 308.3 | N/A | Phenolic acids |
| | (24939-16-0) | | | | |

All the solvents were obtained from E. Merck. The properties are listed in Table 9.

TABLE 9
Physical property of considered organic solvent.

| | | Mw | Density | Tb | Dipole | |
|---|---|---|---|---|---|---|
| Name | Formula | (g/mol) | (g/cm³) | (° C.) | (D) | Acid/base |
| Petroleum | | | 0.791 | 30-60 | 0.0 | — |
| ether | | | | | | |
| hexane | C₆H₁₄ | 86 | 0.655 | 69.0 | 0.0 | — |
| Acetone | C₃H₆O | 58 | 0.791 | 56.2 | 2.88 | pKₐ = 20 |
| Ethanol | C₂H₅OH | 46 | 0.789 | 78.5 | 1.68 | PKₐ = 18 |
| Methanol | CH₃OH | 32 | 0.791 | 64.6 | 2.87 | PKₐ = 16 |

Dowex 50WX2-200 (H) cation exchange resin was purchased from Sigma-Aldrich, Co. It is a strong acid cation exchange resin with 2% cross-linking; hydrogen ion form, 100-200 mesh. Sephadex G-10 (approximate dry bead diameter 40-120 μm) was purchased from sigma-Aldrich, Co. Sephadex is a beaded gel prepared by crosslinking dextran with epichlorohydrin. Its main application is group separation of low and high molecular weight molecules. G-10 is used to separate molecular weight <700.

Analytical Methods

Characterization and Quantification Essential Oil:

The chemical composition of turmeric essential oil was determined with a HP 5890 series GC-MS system equipped with a fused silica column (5% phenylpoly(dimethylsiloxane) XTI-5, 30m×0.25 mm i.d. and 0.25 μm film thickness, Restek). The electron ionization energy was 70 eV. The carrier gas was helium (1.7 ml/min) and 1 μL of sample was injected. The injection temperature was 240° C., and that of the detector was 230° C. The temperature programming was 50° C. for 5 min, increase to 180° C. at 4° C./min and to 280° C. at 15° C./min, and held at 280° C. for 19 min. The identification of compounds was performed by comparing their mass spectra with the data from U.S. National Institute of standards and technology (NIST, USA) and WILEY mass spectral library.

Characterization and Quantification of Curcumionids.

HPLC analysis were performed with a Shimadzu LC-10AVP system including a LC-10ADVP pump, an SPD-M10AVP photodiode array detector, an SCL-10ADVP controller and a CTO-10ACVP column oven using an Jupiter column (250 mm H, 4.6 mm I.D., 5μ C18 300 Å). The elution was carried out with gradient systems with a flow rate of 1 ml/min at 30° C. The mobile phase consisted of 2% acetic acid (A), acetonitrile (B) and methanol (C). Quantitative levels of curcuminoids were determined using the above solvents programmed linearly from 30-36% acetonitrile in A for 0-30 min. The gradient then went from 36% to 95% acetonitrile in A for 30-45 min, with a constant of 5% C. The linearity of the method was evaluated by analyzing a series of standard curcuminoids. 20 μl of each of the five working standard solution containing 0.06-2 μg of standard curcumin, demethoxycurcumin and bisdemethoxycurcumin was injected into HPLC. The standard calibration curves were obtained by plotting the concentration of standard curcuminoids versus peak area (average of three runs). The calibration range was chosen to reflect normal curcuminoid concentrations in turmeric samples.

Characterization and Quantification of Polysaccharides

Colorimetric tests have been used to characterize polysaccharide in curcuma species. Reagent used for test are 95.5% sulfuric acid (conforming to ACS specification, specific gravity 1.84) and 5% phenol solution, prepared by adding 2 g of distilled water to 38 g of reagent grade phenol. This mixture forms a water-white liquid that is readily pipetted. Dextran (Fluka product) with molecular weight of 5220, 48600 and 409800 were used as standard.

2 ml of sugar solution was pipetted into a chlorimetric tube, and 1 ml 5% phenol is added. Then 5 ml of concentrated sulfuric acid is added rapidly, the stream of acid being directed against the liquid surface rather than against the side of test tube in order to obtain good mixing. The tubes are allowed to stand 10 minutes. The color is stable for several hours and readings may be made later if necessary. The absorbance of the characteristic yellow-orange color is measured at 488 nm. Blanks are prepared by substituting distilled water for the sugar solution. The amount of sugar may then be determined by reference to a standard curve constructed for dextran. All solutions are prepared in triplicate to minimize errors resulting from accidental contamination. In order to test the method, the experiments were repeated on different days. In all cases, the variations between experiments were no more than 0.01 to 0.02 units in absorbance, which was the same order of magnitude as the variation between the triplicate samples.

Direct Analysis in Real Time (DART) Mass Spectrometry for Polysaccharide Analysis.

All DART chromatograms were run using the instruments and methods described below.

Instruments: JOEL AccuTOF DART LC time of flight mass spectrometer (Joel USA, Inc., Peabody, Mass., USA). This Time of Flight (TOF) mass spectrometer technology does not require any sample preparation and yields masses with accuracies to 0.00001 mass units.

Methods: The instrument settings utilized to capture and analyze polysaccharide fractions are as follows: For cationic mode, the DART needle voltage is 3000 V, heating element at 250° C., Electrode 1 at 100 V, Electrode 2 at 250 V, and helium gas flow of 7.45 liters/minute (L/min). For the mass spectrometer, orifice 1 is 10 V, ring lens is 5 V, and orifice 2 is 3 V. The peaks voltage is set to 600 V in order to give resolving power starting a approximately 60 m/z, yet allowing sufficient resolution at greater mass ranges. The micro-channel plate detector (MCP) voltage is set at 2450 V. Calibrations are performed each morning prior to sample introduction using a 0.5 M caffeine solution standard (Sigma-Alrich Co., St. Louis, USA). Calibration tolerances are held to ≦5 mmu.

The samples are introduced into the DART helium plasma with sterile forceps ensuring that a maximum surface area of the sample is exposed to the helium plasma beam. To introduce the sample into the beam, a sweeping motion is employed. This motion allows the sample to be exposed repeatedly on the forward and back stroke for approximately 0.5 sec/swipe and prevented pyrolysis of the sample. This motion is repeated until an appreciable Total Ion Current (TIC) signal is observed at the detector, then the sample is removed, allowing for baseline/background normalization.

For anionic mode, the DART and AccuTOF MS are switched to negative ion mode. The needle voltage is 3000 V, heating element 250° C., Electrode 1 at 100 V, Electrode 2 at 250 V, and helium gas flow at 7.45 L/min. For the mass spectrometer, orifice 1 is −20 V, ring lens is −13 V, and orifice 2 is −5 V. The peak voltage is 200 V. The MCP voltage is set at 2450 V. Samples are introduced in the exact same manner as cationic mode. All data analysis is conducted using MassCenterMain Suite software provided with the instrument.

Absorbance Assay:

Protein in solution absorbs ultraviolet light with absorbance maxima at 280 and 200 nm. Peptide bonds are primarily responsible for the absorbance at 200 m. Shimadzu 1700 series spectrophotometer has been used in current research. The procedure include the following steps:
warm up the UV lamp for 15 minutes;
calibrate to zero absorbance with phosphate buffer saline only;
scan sample solution from 190 to 300 nm;
find out the maximum absorbance wavelength.

Bradford Protein Assay:

The Bradford assay can be used to determine the concentration of proteins in solution. The procedure is based on the formation of a complex between the dye, Brilliant Blue G, and proteins in solution. The protein-dye complex causes a shift in the absorption maximum of the dye from 465 to 595 nm. The amount of absorption is proportional to the protein present.

### Reagent:

Bradfrod reagent (sigma product, B6919) consists of 0.004% Brilliant blue G, 10% phosphoric acid and 4% methanol.
Phosphase buffered saline (PH=7.4) (sigma product, P3813) consists of 83.8% sodium chloride, 12% di-sodium hydrogen phosphate anhydrous, 2% monobasic potassium phosphate and 2% potassium chloride.
Bovine serum albumin (BSA) buffered with phosphate saline, PH=7.4: sigma product, P-3688

### Procedure:

Prepare six standard solutions contain 0, 200, 400, 600, 800 and 1000 μg BSA. Set the spectrophotometer to collect the spectra over a wavelength range from 400 to 700 nm and over an absorbance range of 0-2 absorbance units. Use a 4 ml quartz cuvette filled with distilled water to blank spectrophotometer over this wavelength range. Record the absorbance spectrum from 400-700 nm and note the absorbance at 595 nm. Repeat the steps above for each protein standards and for the samples to be assayed. Examine the spectrum of the standards and samples. If any spectrum has an absorbance at 595 nm greater than 2, or if any sample has an absorbance greater than the greatest absorbance for any of the standards, dilute the sample by a known amount and repeat the assay. At one wavelength around 575 nm, all of the spectra should have the same absorbance (such an intersection is called an isosbestic point and is a defining characteristics of solutions containing the same total concentration of an absorbing species with two possible form). If any spectrum does not intersect the other spectra at or near the isosbestic point, it should be adjusted or rejected and repeated.

Prepare graph of absorbance at 595 nm vs BSA concentration. To determine the protein concentration of a sample from it absorbance, use the standard curve to find the concentration of standard that would have the same absorbance as the sample.

### Thioflavin T Assay

The presence of Aβ₁₋₄₂ fibers was monitored by thioflavin T fluorescence. Triplicate 15 L samples of Aβ₁₋₄₂ [50 μM in 50 mM Tris-HCl buffer (pH 7.4) were removed after incubation of the peptide solution for various period of time at 37° C. in the presence or absence of a curcuma extract of the present invention or control compound at different doses. These samples were each added to 2 mL of 10 μM thioflavin T (Sigma) in 50 mM glycine/NaOH (pH 9.0) before the characteristic change in fluorescence was monitored (excitation at 450 nm and emission at 482 nm) following binding of thioflavin T to the amyloid fibers. Triplicate samples were scanned three times before and immediately after the addition of peptide. Results show the mean value of the triplicate samples ± the difference between those mean values.

### Aβ_{1-40, 42} ELISA

Conditioned media were collected and analyzed at a 1:1 dilution using the method as previously described (Tan et al., 2002) and values were reported as percentage of Aβ₁₋ₓ secreted relative to control. Quantitation of total Aβ species was performed according to published methods (Marambaud et al., 2005; Obregon et al., 2006). Briefly, 6E10 (capture antibody) was coated at 2 μg/mL in PBS into 96-well immunoassay plates overnight at 4° C. The plates were washed with 0.05% Tween 20 in PBS five times and blocked with blocking buffer (PBS with 1% BSA, 5% horse serum) for 2 hours at room temperature. Conditioned medium or Aβ standards were added to the plates and incubated overnight at 4° C. Following 3 washes, biotinylated antibody, 4G8 (0.5 μg/mL in PBS with 1% BSA) was added to the plates and incubated for 2 hours at room temperature. After 5 washes, streptavidin-horseradish peroxidase (1:200 dilutions in PBS with 1% BSA) was added to the 96-wells for 30 minutes at room temperature. Tetramethylbenzidine (TMB) substrate was added to the plates and incubated for 15 minutes at room temperature. 50 μL of stop solution (2 N N₂SO₄) was added to each well of the plates. The optical density of each well was immediately determined by a microplate reader at 450 nm. Aβ levels were expressed as a percentage of control (conditioned medium from untreated N2a SweAPP cells).

### Example 1

### Example of Single Step SCCO2 Extraction

The extraction was carried out using a SFT-250 SFT/SFR Processing Platform, Supercritical Fluid Technologies, Inc., Newark, Del. The curcuma species essential oil fraction was extracted with SCCO2 in a semi-continuous flow extraction process. Liquid carbon dioxide from a storage cylinder was passed through a cooling bath and was then compressed to the operating pressure by an air-driven Haskel pump. Compressed carbon dioxide flowed into the 100 ml extraction vessel containing 30 gm ground curcuma species rhizome powder (20 mesh) up to a point where no solute was observed at the exit of the extraction vessel. The extraction vessel containing the raw plant material to be extracted was in a thermostatically controlled oven. The temperature inside the extraction vessel was controlled with a digital controller within an accuracy of +/−0.1° C. The flow rate of the carbon dioxide was 10 L/min (19 gm/min). The volume of carbon dioxide consumed was calculated with flow rate and running time. The extraction products were collected into 5 fractions for each run at definite time intervals in a glass ampoule 65 mm high and 24 mm in diameter, and weighed gravimetrically to obtain extraction curves. The experiments were run at a pressure of 300 bar and temperature of 40° C. The amount of carbon dioxide soluble material extracted was calculated as the ratio of total mass weight of the extract and the total mass weight of the natural feedstock material. The extraction products were dissolved in hexane for Gas Chromatography-Mass Spectroscopy (GC-MS) analysis. The results are shown in Tables 2 and 6. There was a high total yield of 4.2% by mass weight based on the weight of the original curcuma feedstock and the high concentration of the three principal turmerones, ar-turmerone, β-turmerone, and α-turmerone which make up 76.5% of the essential oil fraction by % mass weight. The purity of the essential oil chemical constituents was greater than 99%.

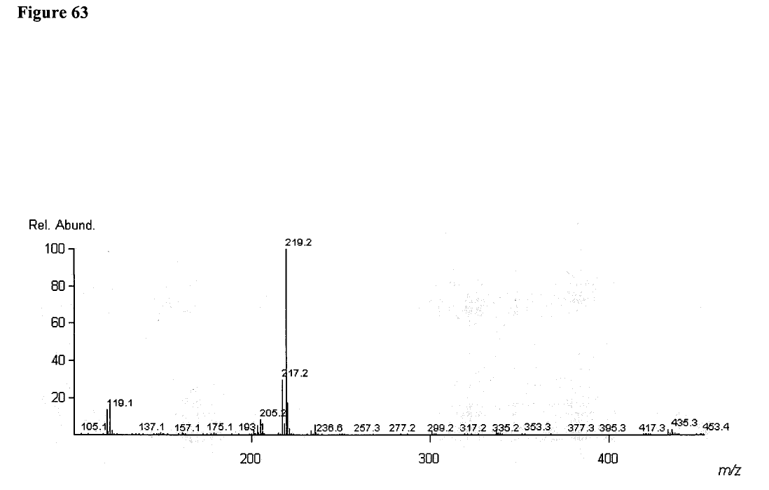

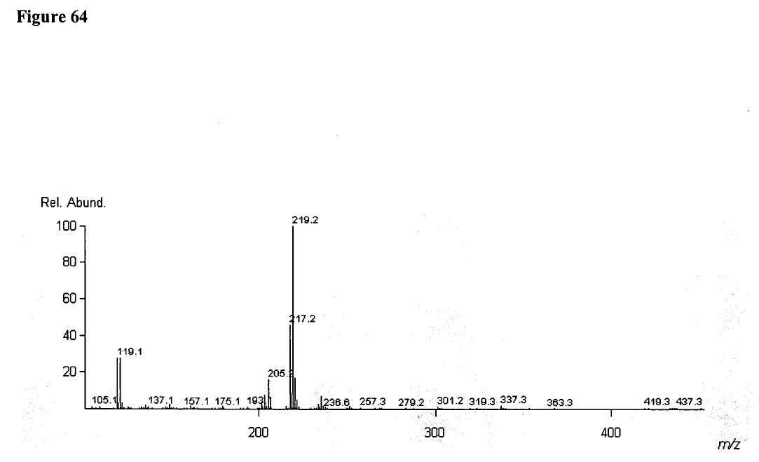

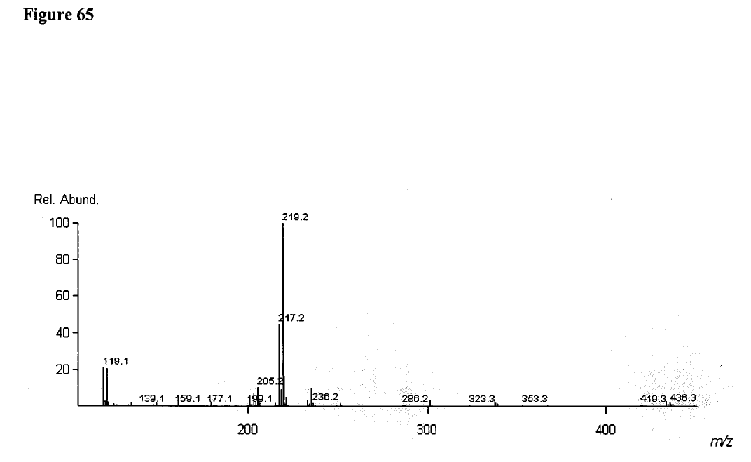

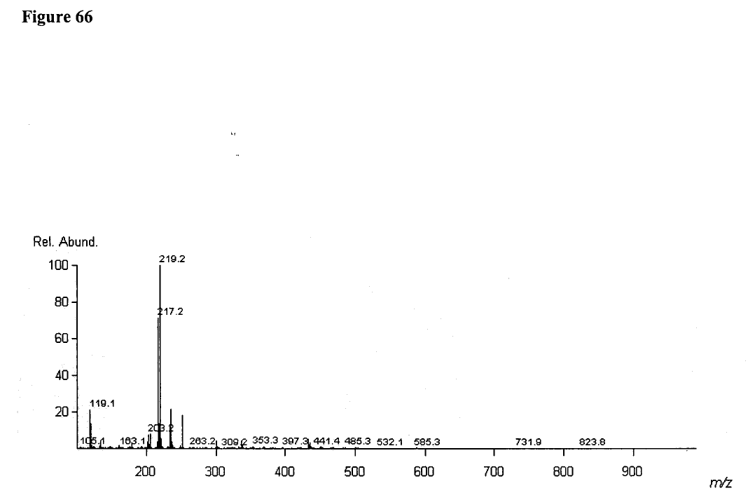

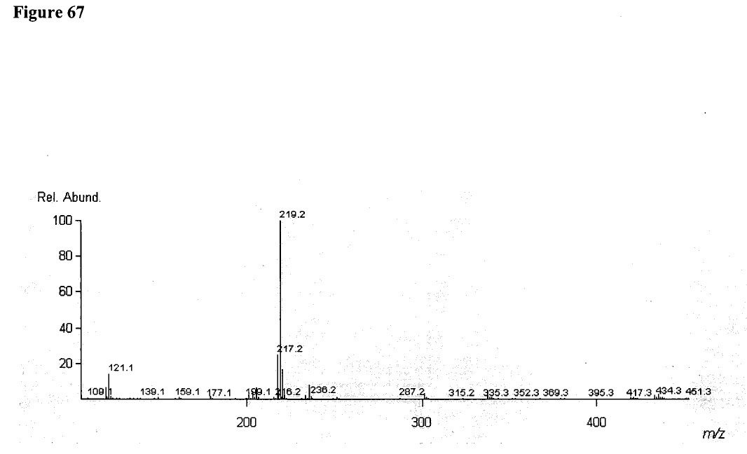

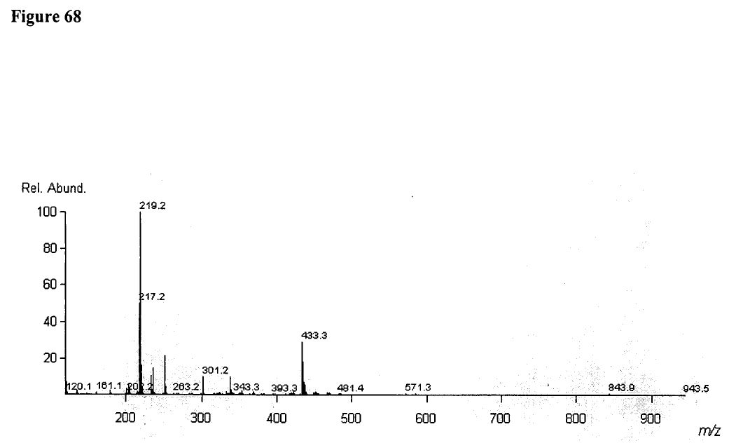

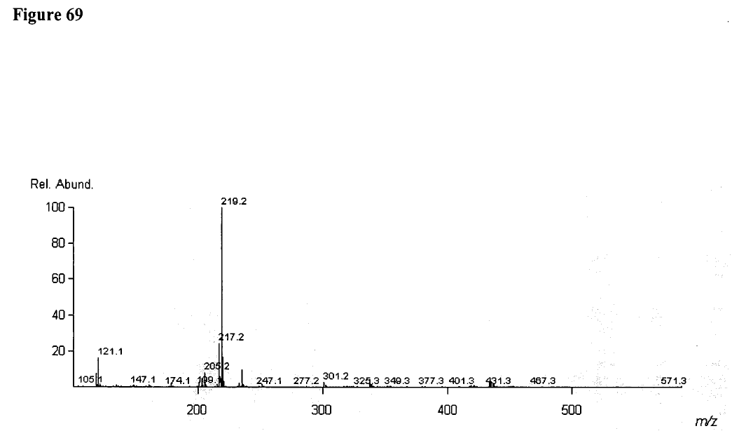

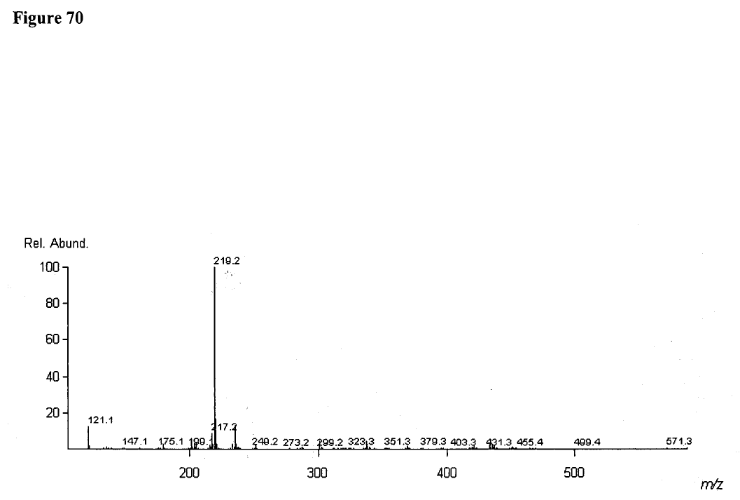

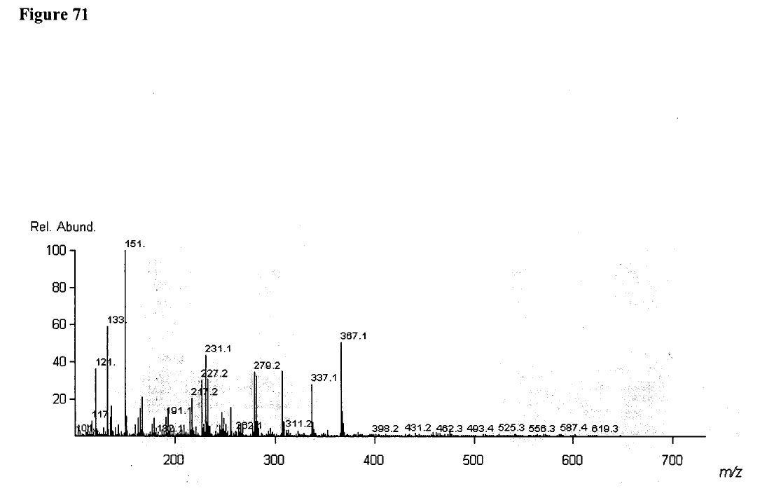

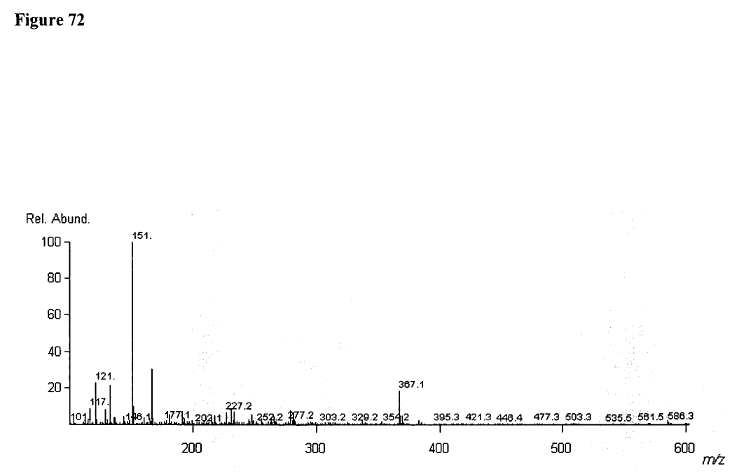

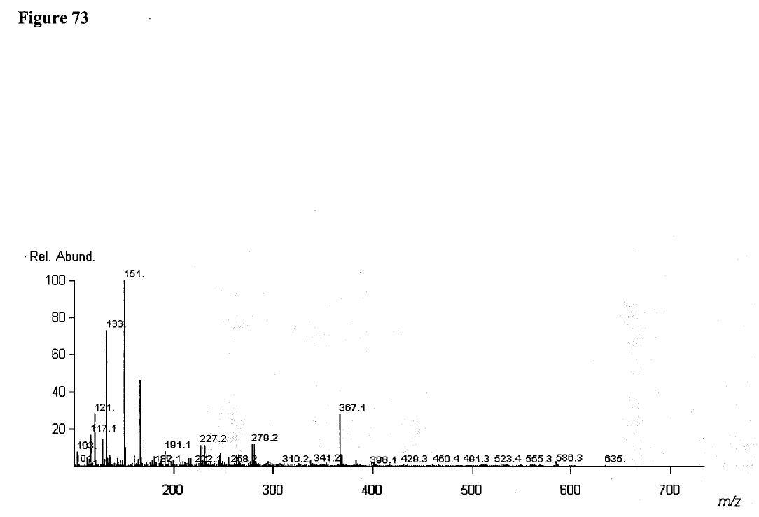

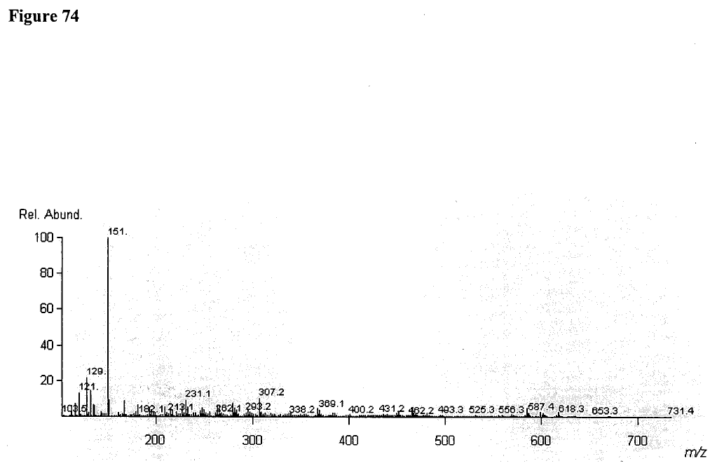

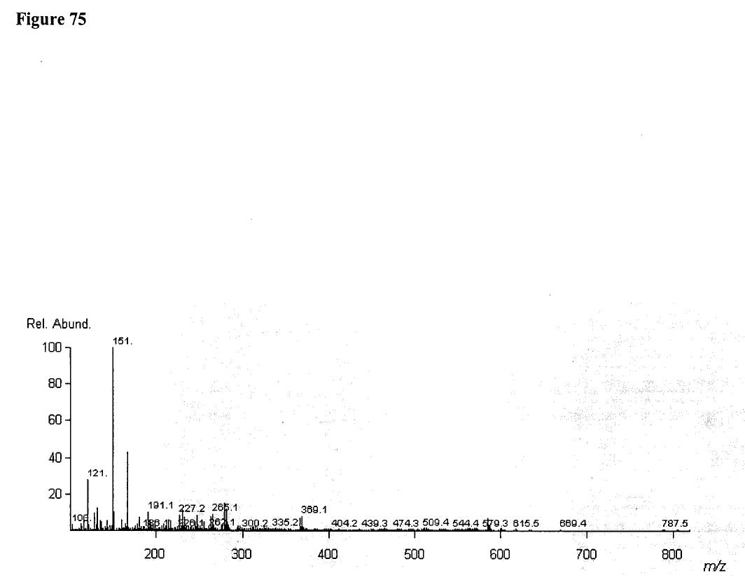

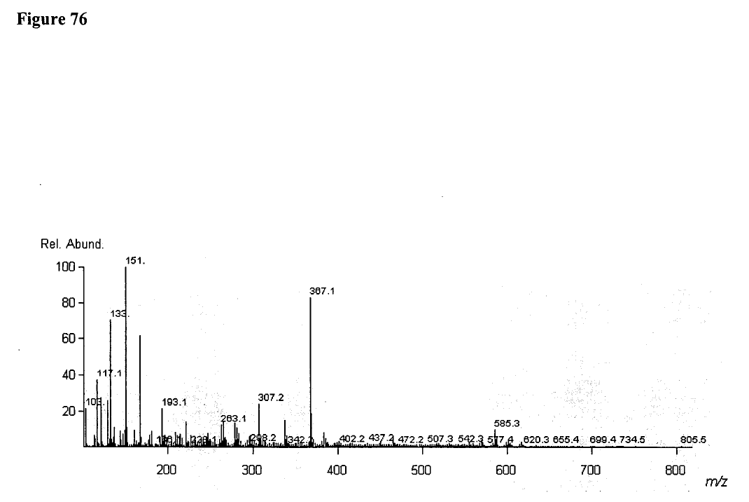

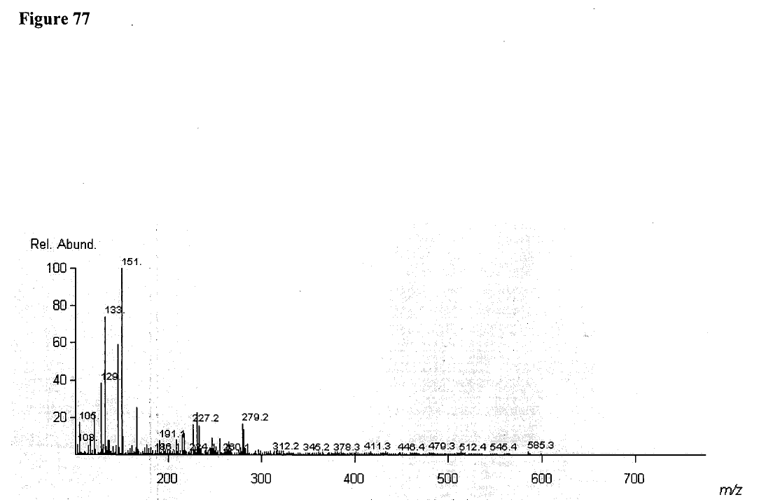

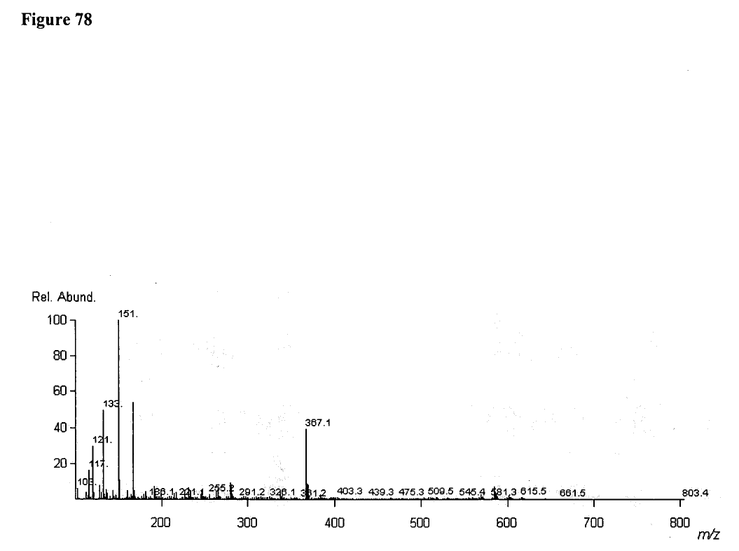

The above procedure was run several times with varying temperatures and pressures. Fractions from these runs were collected and analyzed by DART mass spectrometry and appear in FIGS. 63-78.

### Example 2

### Example of SCCO2 Extraction and Fractionation

SCCO2 extraction and fractionation of the curcuma species feedstock was performed using a proprietary supercritical fluid extraction and fractionation system as previously described. 2,000 gm of the ground curcuma species rhizome feedstock was introduced into the 24 L extraction vessel. The extraction temperature and pressure were adjusted and the carbon dioxide feed was started. The compressed CO2 was allowed to flow upwards through a vertically mounted bed, and the essential oil and other lipophilic chemical constituents including the curcuminoids were extracted. The solution exited the extractor vessel through a pressure-reducing valve and flowed into the first separator, where and carbon dioxide was evaporated and recycled. Stagewise precipitation of the extracts was accomplished by releasing the solvent pressure and decreasing the temperature in three stages using the three fractionation separators in series. Separators 1 and 3 were used for fractionation. The heavy extraction product (curcuminoid fraction) precipitated in the separator 1 collection vessel at a higher pressure, the light product (essential oil) was recovered in the separator 3 at a lower pressure. The total weight of CO2 consumed and the flow rate of the fluid were measured by mass flow meter and flow time. Pressure was set by automatic back pressure valve with an accuracy of +/−3 bar in the extractor vessel and of +/−1 bar in the separator vessels. The temperature was adjusted with thermostats with an accuracy of +/−1° C. The extraction temperature and pressure was as follows: 70° C. and 450 bar for the extraction vessel; 65° C. and 170 bar for Separator 1; 59° C. and 130 bar for Separator 2; and 28° C. and 60 bar for Separator 3. The SCCO2 extraction conditions and yields (% mass weight based on the feedstock) are documented in Table 8.

The essential oil was collected in Separator 3. GC-MS analytical results are shown in Tables 6 & 7. Using the above SCCO2 conditions for fractional separation, 95.5% of the essential oil in the feedstock can be extracted in 30 minutes of extraction time. In this highly purified (>99%) essential oil fraction, three chemical constituents, ar-turmerone, α-turmerone, and β-turmerone, comprised 73.6% by mass weight.

TABLE 10
Peak area % of turmeric essential oil extracts by different solvent.

| | | | | | | | | CO2 @ | CO2 @ |
|---|---|---|---|---|---|---|---|---|---|
| | retention time | | | | | | | 40 C. | 70 C. and |
| Peak # | (min) | peak ID | Mw | Hexane | Et-Ac | Acetone | ethanol | and 300 bar | 450 bar |
| 1 | 30.36 | α-Curcumene | 202 | 2.0 | 8.8 | 9.2 | 10.5 | 2.1 | 1.5 |
| 2 | 30.76 | (−)-Zingiberene | 204 | 2.5 | 11.5 | 12.1 | 17.7 | 2.1 | 1.4 |
| 3 | 31.69 | β-Sesquiphellandrene | 204 | 3.2 | 15.6 | 17.5 | 18.8 | 2.7 | 2.0 |
| 4 | 33.46 | Benzene, 1-methyl-4-(1-methylethyl)- | 134 | | | | | 0.6 | 0.4 |
| 5 | 34.23 | Benzene, 1-methyl-2-(1-methylethyl)- | 134 | 0.8 | 1.7 | | | 0.5 | 0.9 |
| 6 | 34.98 | Benzene, 4-ethyl-1,2-dimethyl- | 134 | | | | | 0.6 | 1.1 |
| 7 | 35.21 | Cyclohexene, 1-(1-propynyl)- | | 0.4 | 14.25 | | | | 0.7 |
| 8 | 35.96 | ar-tumerone | 218 | 24.4 | 17.4 | 13.3 | 13.6 | 34.9 | 1.3 |
| 9 | 36.43 | β-tumerone | 218 | 36.6 | 16.3 | 19.7 | 15.6 | 21.4 | 54.2 |
| 10 | 37.07 | Compound 1 | | | | 18.1 | 16.4 | 0.8 | 0.7 |
| 11 | 37.36 | α-tumerone | 216 | 24.2 | 8.4 | 8.3 | 7.4 | 20.2 | 18.2 |
| 12 | 38.33 | (6S,1′R)-6-(1′5′-dimethylenex-4′-enyl)-3- | 220 | | | | | | |
| | | methylcyclohex-2-enone) | | 0.9 | 1.3 | 8.0 | | 1.1 | 1.0 |
| 13 | 38.57 | Compound 2 | 216 | 1.1 | 1.9 | | | 1.2 | 1.1 |
| 14 | 38.74 | (+)-beta-atlantone | | 4.0 | 2.8 | | | 1.0 | 0.9 |
| 15 | 38.84 | Compound 3 | | | | | | 1.2 | 1.2 |
| 16 | 38.94 | Compound 4 | | | | | | 2.6 | 2.1 |
| 17 | 39.24 | Compound 5 | | | | 1.9 | | 0.5 | 0.9 |
| 18 | 39.41 | (+)-alpha-atlantone | | | | | | 0.8 | 1.6 |
| 19 | 39.64 | Compound 6 | | | | | | 1.3 | 1.4 |
| 20 | 39.76 | 3-buten-2-one, 4-(4-hydroxy-3-methoxyphenyl)- | 234 | | | | | 1.2 | 1.8 |
| 21 | 40.04 | Compound 7 | | | | | | 0.6 | 0.2 |
| 22 | 40.74 | Compound 8 | 230 | | | | | 0.4 | 1.6 |
| 23 | 41.02 | Compound 9 | | | | | | 0.4 | 0.9 |
| 24 | 41.43 | Hexdecanoic acid, methyl ester | | | | | | 1.6 | 0.6 |
| 25 | 42.05 | Pentadecanoic acid, 14-methyl-, methyl ester | | | | | | | 0.9 |
| 26 | 42.89 | 9,12-Octadecadienoic acid, methyl ester, (E,E)- | 295 | | | | | | 1.3 |

| | | | | | | |
|---|---|---|---|---|---|---|
| Turmerone Percentage In extracts (%) | 85.2 | 40.1 | 41.3 | 36.5 | 76.5 | 73.6 |

TABLE 11
Total yield, three turmerone distribution expressed by peak percentage.

| | | | | | | Total |
|---|---|---|---|---|---|---|
| | | Total | ar- | β- | α- | turmerone |
| SCCO2 | Density | yield | turmerone | Turmerone | turmerone | purity |
| Conditions | (g/cm³) | (%) | (%) | (%) | (%) | (%) |
| CO2@40° C. and 300 bar | 0.909 | 4.2 | 34.9 | 21.4 | 20.0 | 76.5 |
| CO2@70° C. and 450 bar | 0.812 | 5.96 | 1.3 | 54.2 | 18.2 | 73.6 |
| and fraction at 28° C. | | | | | | |
| and 60 bar | | | | | | |

TABLE 12
SCCO2 extraction/fractionation experiment run conditions and yield.
The yield was calculated by extracts/feedstock

| Feed (g) = 2000 | | | |
|---|---|---|---|
| Flowrate (kg/min) = 1.5 | T (° C.) | P (bar) | Yield (%) |
| Extractor | 70.1 | 450 | 2.35 |
| Step 1 | 65 | 170 | 1.80 |
| Step 2 | 59 | 130 | 0.33 |
| Step 3 | 28.4 | 60 | 1.18 |

A highly purified (81.3%) curcuminoid fraction was collected in Separator 1 in the above experimental example (the yield in Separator 2 was only 0.02% without any significant amount of either essential oil or curcuminoid chemical constituent being present and was therefore discarded). The total yield was 1.80% mass weight based on the feedstock with an 81.3% concentration of the curcuminoids. The yield of the curcuminoids was 22% mass weight based on the feedstock. However, there remained 78% of the curcuminoids (5.42% mass weight) remaining in the SCCO2 residue which was addressed in Step 2 below. The HPLC analytical results of the Separator 1 extracted curcuminoid fraction are documented in Table 13.

TABLE 13
Separator 1 Curcuminoid Extraction Fraction
Separator 1

| Cur | Cur | | | |
|---|---|---|---|---|
| yield | purity | C | DMC | BDMC |
| (%) | (%) | (%) | (%) | (%) |
| 22.1 | 81.3 | 81.0 | 14.8 | 4.2 |

Cur = curcuminoids;
C = curcumin;
DMC = demethoxycurcumin;
BDMC = bisdemethoxycurcumin.

### Example 3

### Example of Step 2 Extraction

400 gm of SFE Step 1 residue was loaded with 95% ethanol into an extraction vessel and mixed for 5 hours at 75° C. The mixing was then discontinued and the solution was allowed to stand for 16 hours. The top layer was decanted and filtered 2 times with Fisherbrand P4 filter paper with 4-8 μm particle retention size and centrifuged at 3000 rpm. The curcuminoid enriched supernatant was evaporated and either vacuum oven dried at 50° C. to a tart or spray dried into a dry flowable powder. This dried extraction product was then used for further processing (Step 3). HPLC analysis and data are shown in Table 14. The total yield of the leaching process was 4.52% weight based on the original curcuma feedstock with a curcuminoid purity of 37.8%. The curcuminoid distribution or profile by % mass weight of the curcuminoids was curcumin 35.1%, bisdemethoxycurcumin 39.0%, and demethoxycurcumin 25.9%. This extraction process was capable of extracting 83.3% of the curcuminoid chemical constituents in the SFE residue feedstock with a total yield of 11.9% mass weight based on the original native curcuma species feedstock. The solid residue (bottom layer) was saved for further processing to obtain purified fractions of curcuma peptide proteins and curcuma polysaccharides (Steps 5, 6, & 7).

TABLE 14
Leaching process yield and curcuminoid purity in extracts by using Step 1 SFE
residue as feedstock.

| | | | | | | Curcuminoid yield based on | | | |
|---|---|---|---|---|---|---|---|---|---|
| Total | Curcuminoid | | Curcuminoid | | | chemical composition | | | |
| yield | yield based on | Curcuminoid | distribution (%) | | | in feedstock (%) | | | |
| (%) | feedstock (%) | purity (%) | BDMC | DMC | C | BDMC | DMC | C | Total |
| 11.9 | 4.52 | 37.8 | 39.0 | 25.9 | 35.1 | 100 | 100 | 47.7 | 83.3 |

### Example 4

### Example of SCCO2 Purification of Ethanol Leaching Product

A 300 gm portion of the curcuminoid enriched ethanol leaching process extraction product was mixed with 2,000 gm glass beads (O.D.=80 mm) and then loaded into the 24 L SFE extraction vessel. Once the extraction temperature and pressure were adjusted, the carbon dioxide flow was started. The compressed CO2 was allowed to flow upwards through the vertically mounted bed in the extraction vessel. Lipophilic substances such as the curcuminoids were extracted. The solution leaves the extraction vessel through a pressure reducing valve and flowed into the Separator 1 where the CO2 was evaporated for recycling. Stagewise precipitation of the extract solution were accomplished by releasing the solvent pressure and temperature in three stages using the three separators in series. After reducing the pressure and temperature in Separator 1, the heavy product that contains the curcuminoids precipitated in Separator 1. Lighter products made up of lipophilic chemical constituents which were essentially free of curcuminoids on HPLC analysis were recovered in Separators 2 & 3 and were discarded. The flow rate of the fluid was measured to be 3.5 kg/min. using a mass flow meter. The total running time was 120 minutes and the solvent/feed ratio was 426. The conditions for the extraction vessel were a pressure of 400 bar and a temperature of 90° C. The pressures and temperatures for the Separators were set as follows: Separator 1-170 bar, 63° C.; Separator 2-130 bar. 65° C.; and Separator 3-60 bar, 28° C. The Separator 1 extraction product results are shown in Table 15. In order to further purify and profile curcuminoid chemical constituents of this extraction, an additional SCCO2 extraction and fractionation step (Step 4) was required.

TABLE 15
SCCO2 process (Step 3) yield and curcuminoid purity in extraction product using
Step 2 ethanol leaching extraction of Step 1 SFE residue as feedstock.

| | | | | | | | Curcuminoid yield based on | | | |
|---|---|---|---|---|---|---|---|---|---|---|
| | Total | Curcuminoid | Curcuminoid | Curcuminoid | | | chemical composition | | | |
| | yield | yield based on | purity | distribution (%) | | | in feedstock (%) | | | |
| Extract | (%) | feedstock (%) | (%) | BDMC | DMC | C | BDMC | DMC | C | Total |
| Separator 1 | 2.63 | 2.32 | 88.1 | 8.2 | 19.8 | 72.0 | 4.08 | 14.8 | 39.8 | 51.4 |

### Example 5

### Example of Purification and Profiling of Step 3 Curcuminoid Extraction Product

10 gm of the extraction product of Step 3 was mixed with 40 ml (45 gm) of glass beads (diameter=4 mm) and then loaded into an 1 L extraction vessel 1 of a proprietary HerbalScience designed 1 L laboratory scale SFE fractionation system modeled on the 24 L production scale system. After purge and leak testing, the extraction vessel was brought up to a pressure of 413 bar and temperature of 90° C. Two Separators were used for the fractionation. Separator 1's temperature and pressure were set at 65° C. and 170 bar and Separator 2's temperature and pressure were set at 65° C. and 130 bar. Once the system reached equilibrium at the set conditions, carbon dioxide flow at a flow rate of 40 L/min from bottom to the top of a vertically mounted feedstock bed in the extraction vessel. The total carbon dioxide flow time was 120 minutes with a solvent to feed ratio of 705. The fractions extracted in each of the Separators were analyzed using HPLC for identification of the curcuminoid chemical constituents and calculations of the purity of these components. The results are shown in Table 16.

TABLE 16
SCCO2 purification and profiling of Step 3 extraction product.

| | | Curcuminoid | | | | | | | | |
|---|---|---|---|---|---|---|---|---|---|---|
| | | yield | | | | | Curcuminoid yield based on | | | |
| | Total | based on | | Curcuminoid | | | chemical composition | | | |
| | yield | feedstock | Curcuminoid | distribution (%) | | | in feedstock (%) | | | |
| | (%) | (%) | purity (%) | BDMC | DMC | C | BDMC | DMC | C | Total |
| Feedstock | 2.63 | 2.32 | 88.0 | 5.0 | 22.2 | 72.9 | | | | |
| Separator 1 | 0.89 | 0.84 | 94.7 | 5.1 | 19.3 | 75.6 | 0.98 | 0.83 | 0.99 | 0.95 |
| Separator 2 | 0.71 | 0.30 | 42.8 | 5.5 | 18.2 | 76.3 | 0.39 | 0.28 | 0.36 | 0.36 |

### Example of Purification and Profiling of an Enriched Curcuminoid Extraction Product

In another example of purification and fractional profiling of the curcuminoids, a highly curcuminoid concentrated extraction product (Lot #: CL02005) purchased from Suan Farma, Inc. was used as feedstock. In this feedstock extract, the total curcuminoid concentration was 91.14% by mass weight with a curcuminoid distribution as follows: curcumin (C) 68.22%; demethoxycurcumin (DMC) 9.63%; and bisdemethoxycurcumin (BDMC) 4.81%. 300 gm of this extraction product mixed with 1,200 gm of glass beads (O.D.=1 cm) was loaded into the 24 L extraction vessel. The extraction temperatures and pressures were adjusted and then the carbon dioxide feed was initiated. The compressed carbon dioxide was allowed to flow upwards through a vertically mounted bed of feedstock in the extraction vessel and lipophilic chemical constituents including the curcuminoids were extracted. Every 30 minutes, 1.38 L ethanol co-solvent was added from the bottom of the extractor vessel by using a high pressure Haskel liquid pump and let it sit for 5 minutes before initiating dynamic CO2 flow. The extraction solution left the extraction vessel through a pressure reducing valve and flowed into Separator 1 where the carbon dioxide was evaporated for recycling. Stagewise p precipitation of the extract was accomplished by reducing the pressure and temperature in three stages using the three Separators in series. After reducing the pressure, the heaviest chemical constituents precipitated into Separator 1 and the lighter chemical constituents in Separators 2 and 3. The total weight of carbon dioxide consumed was measured by mass flow meter and flow time. Pressure was set by an automatic back pressure valve with an accuracy of +/−3 bar for the extraction vessel and of +/−1 bar for the Separator vessels. The temperatures were adjusted using thermostats with an accuracy of +/−1° C. The flow rate of the fluid was measured using a mass flow meter. The processing time was 2 hours with a CO2 flow rate of 3.5 kg/min. A volume of 5.5 L of absolute ethanol was used as a co-solvent. The ethanol co-solvent was phase separated from the CO2 in Separator 3 and was pull out of the system every 30 minutes to avoid ethanol accumulating in the system. The ethanol was recycled via distillation. The results of this example extraction are shown in Tables 17 & 18.

TABLE 17
SCCO2 extraction/fractionation conditions and yield
for Suan Farma feedstock

| Feed (g) = 300 | | | |
|---|---|---|---|
| Flowrate (kg/min) = 3.55 | | | |
| S/F = 1420 | | | |
| Cosolvent (%) = 1.0 | T (° C.) | P (bar) | Yield (%) |
| Extractor | 90.0 | 600 | 65.6 |
| Step 1 | 63.0 | 170 | 60.3 |
| Step 2 | 61.0 | 130 | 1.0 |
| Step 3 | 26.0 | 60 | 4.3 |

TABLE 18
SCCO2 Separator 1 extraction/profiling extraction product for
Suan Farma feedstock.

| | Sep 1 | | | | |
|---|---|---|---|---|---|
| | Cur | Cur | | | |
| | yield | purity | C | DMC | BDMC |
| feedstock | (%)* | (%)** | (%) | (%) | (%) |
| extracts | 64.40 | 97.3 | 86.2 | 11.2 | 2.6 |

*yield mass weight/feedstock mass weight.
**curcumin mass weight/yield mass weight.

### Example 6

### Example of Water Leaching of Step 2 Residue

30 gm Curcuma ethanol extraction residue (Step 2) was loaded in an open flask for 3 hours at 90° C. with 20 volumes of distilled water with constant magnetic stirring. The slurry was centrifuged for 15 minutes at 3000 rpm. The supernatant was collected. The total dry mass weight yield was 9.9% based on the original feedstock. Rotary evaporation was used to evaporate the water and concentrate the extract by about 60%. The solid residue was discarded. Analytical results are list as “crude” in Table 19.

TABLE 19
Yield of curcuma species water extracts precipitated by ethanol and polysaccharide
analysis.

| | | | | | | | | | Purity calculated by dextran | | | |
|---|---|---|---|---|---|---|---|---|---|---|---|---|
| | | | | | Dextran equivalents (μg) | | | | eq. (%) | | | |
| | Mass | Yield | UV | Sample | Low | | | | Low | | | |
| | (g) | (%) | absorb | (ug) | fraction | 5K | 50K | 410K | fraction | 5K | 50K | 410K |
| crude | 1.837 | 19.8 | 0.287 | 19.91 | 5.6 | 6.1 | 7.1 | 5.2 | 28.3 | 30.6 | 35.5 | 26.0 |

### Example 7

### Example of Step 6 Polysaccharide Fraction and Purification

The concentrated supernatant solution from Step 5 was diluted adding sufficient ethanol to make a final 60% ethanol/water concentration solution. This results in precipitation of the water soluble, ethanol insoluble polysaccharides. The solution was then centrifuged at 3000 rpm for 15 minutes and then decanted from the precipitate. The residue solution was saved for further processing to obtain a purified turmerin (peptide) fraction (Step 7). The precipitate yield was 6.4% mass weight based on the original curcuma species feedstock. The ethanol and water remaining in the precipitate was removed using rotary evaporation. The dried precipitate was measured for polysaccharide content using a colormetric method. The results are found in Table 15. The polysaccharide precipitation using a 60% ethanol/water solution was chosen as higher concentrations of ethanol did not substantially add to the yield of polysaccharide precipitate. Furthermore, UV scanning from 190-300 nm of the of the residue solution revealed that the maximum absorbance at about 202 nm (absorbance due to turmerin peptide bonds) disappeared in 80% ethanol/water solutions or higher concentrations indicating that the peptide, turmerin was being precipitated at these ethanol concentrations.

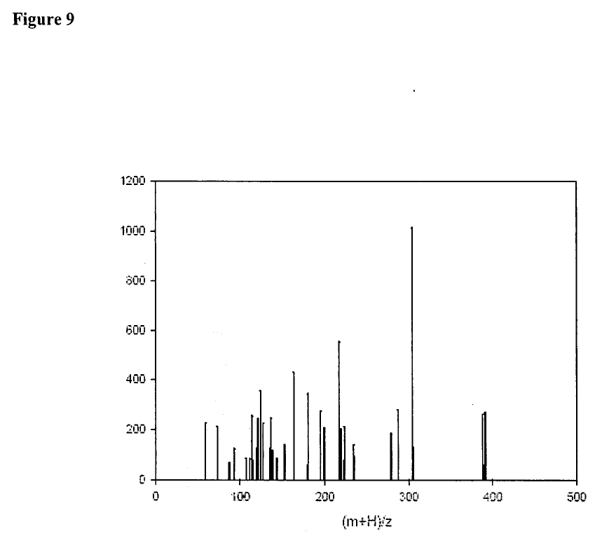

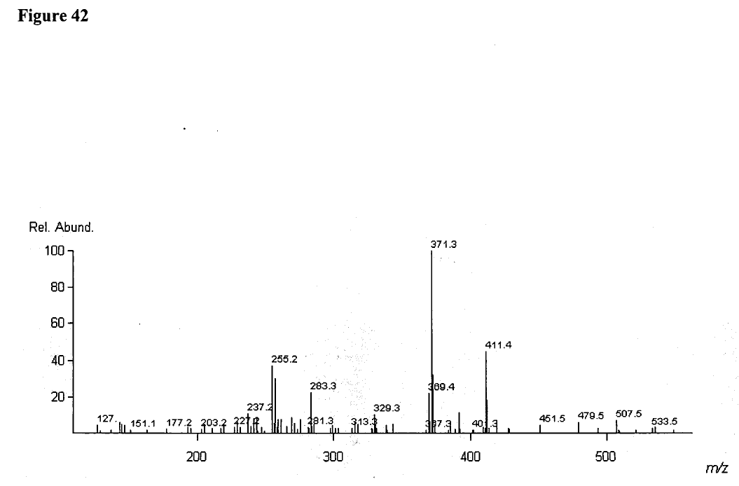

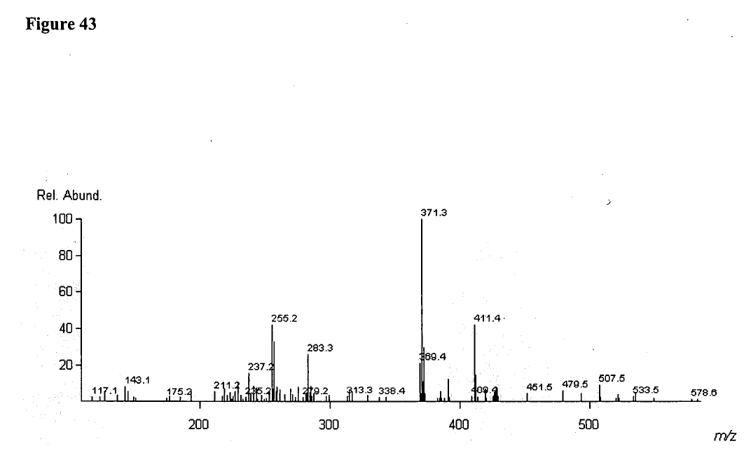

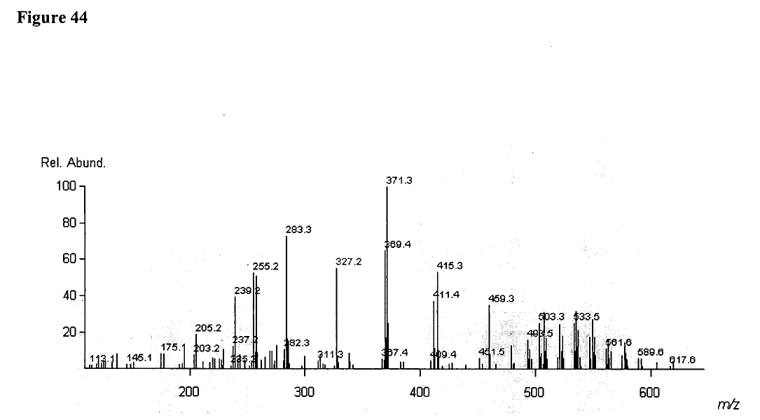

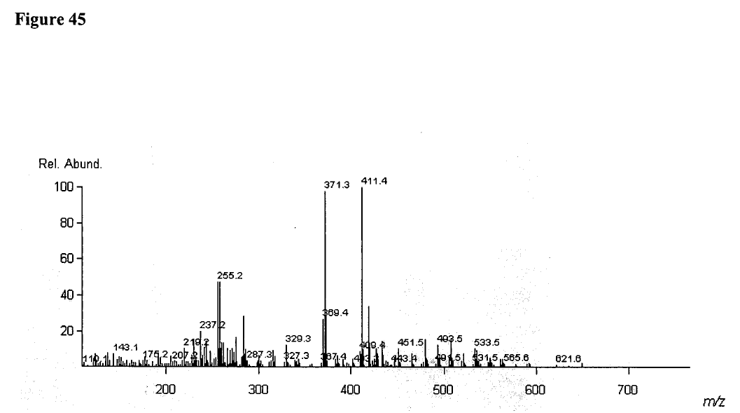

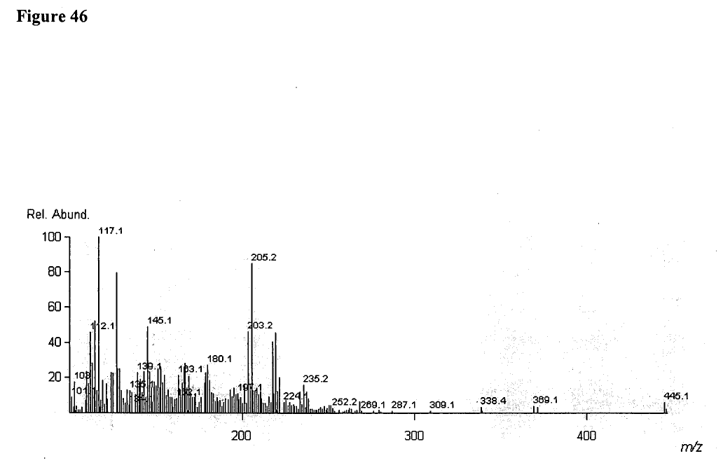

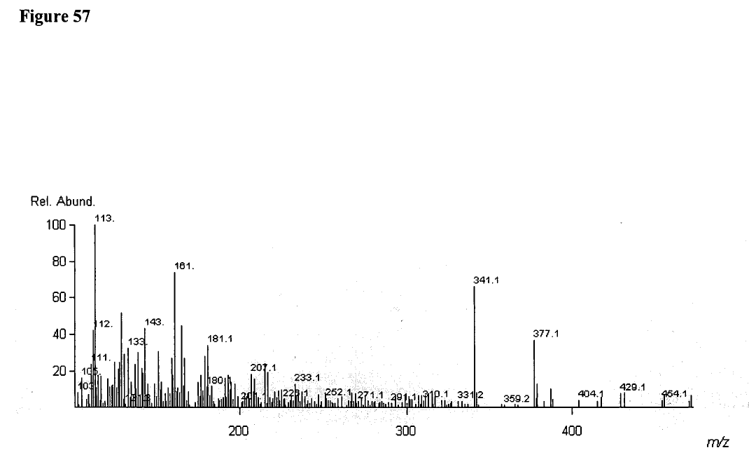

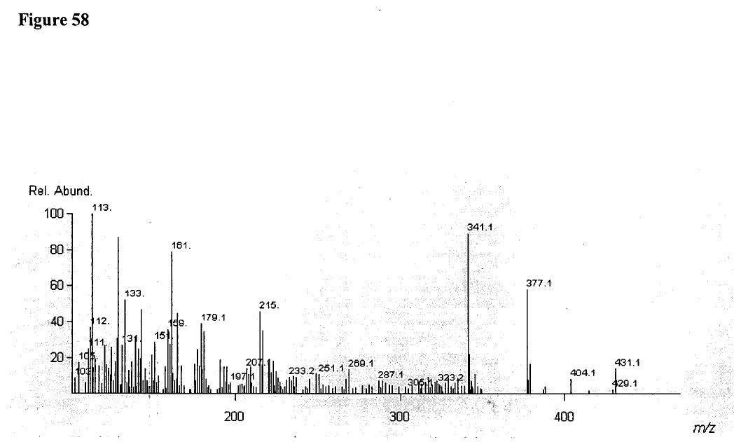

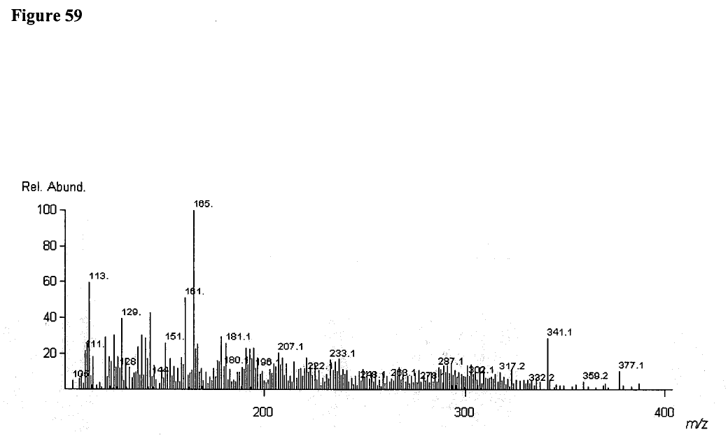

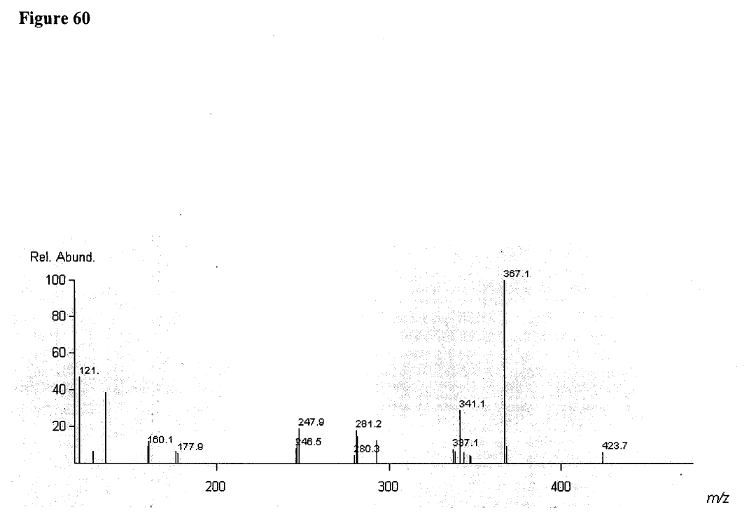

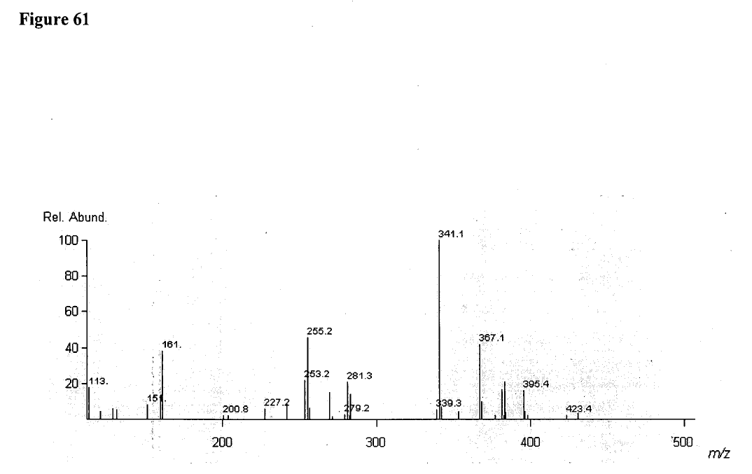

In order to further purify the polysaccharide fraction obtained by 60% ethanol precipitation, a Sephadex G-10 column was used. Sephadex G-10 consists of small, porous, spherical beads of cross-linked dextran molecules. Sephadex G-10 was supplied from Sigma-Aldrich Co. (St. Louis, Mo.) in the form of spherical beads, 10-40 μm diameter. When suspended in water, pores in the material will admit molecules with molecular weights less than 700. The Sephadex beads were hydrated for 16 hours with distilled water. The column was prepared by the addition of the Sephadex suspension to make a bed of 30 ml. The precipitated polysaccharide was dissolved in distilled water to a concentration of 1% by mass weight and loaded onto the column. The feedstock loading flow rate is about 1.8 bed volume/hour. The effluent was collected and measure for polysaccharide content. The results of the colormetric analysis are shown in Table 20. Moreover, AccuTOF-DART mass spectrometry was used to further profile the molecular weights of the compounds comprising the polysaccharide fractions. The results are shown in FIGS. 9, 10, 42-46, and 57-61. These data indicate that the Sephadex G-10 column can purify the curcuma species polysaccharide fraction to a level of about 92% with a 4.5% yield by weight based on the original feedstock.

TABLE 20
Polysaccharide analysis for water extracts and 60% ethanol precipitates using ethanol
extraction residue as feedstock (Step2).

| | | | | | Dextran equivalent ((μg) | | | | Purity by dextran (%) | | | |
|---|---|---|---|---|---|---|---|---|---|---|---|---|
| | Mass | Yield | UV | Sample | Low | | | | Low | | | |
| sample | (g) | (%) | absorb | (μg) | fraction | 5K | 50K | 410K | fraction | 5K | 50K | 410K |
| Crude | 3.1 | 9.9 | 0.25 | 19.91 | 4.7 | 5.2 | 5.9 | 4.4 | 23.57 | 26.06 | 29.60 | 21.87 |
| 60% | 1.9 | 6.2 | 0.59 | 19.91 | 13.4 | 13.5 | 16.6 | 11.9 | 67.19 | 67.57 | 83.56 | 59.97 |
| EtOH | | | | | | | | | | | | |
| G-10 | 1.4 | 4.5 | 0.640 | 19.67 | 14.7 | 14.7 | 18.2 | 13.1 | 74.47 | 74.54 | 92.57 | 66.34 |

### Example 8

### Example of Step 7 Turmerin Fraction Extraction and Purification

The supernatant residue solution from a Step 6 polysaccharide fraction extraction was found to have a 0.3% mass weight concentration (1.3 gm solid in 438.9 gm of the ethanol/water solution). The concentration concentrated solution was then diluted with a phosphate buffered saline solution (0.01M NaCl, 0.0027M KCl, 7.4 pH, 25° C.) to a final concentration of 1 mg/ml (total solution=1300 ml). This solution was then purified by Sephadex G-10 column and Dowex cation exchange column.

Sephadex G-10 beads were soaked in 200 ml of distilled water for 16 hours. The water was decanted and the beads were mixed with fresh distilled water to make a slurry. The column was packed with a 30 ml bed of the Sephadex slurry. A volume of 175 ml of the 1 mg/ml solution was loaded into the column over 12 hours (14.6 ml/h). The effluent was collected. Mass analysis demonstrated that 14.5% solid was removed during this step leaving 0.150 gm of solid in solution.

Dowex 50-WX2-200 strong acid cation exchange resion beads which have sulfonic acid (—SO₃H) groups as exchange groups was used for further purification of the effluent solution. The Dowex resin beads were washed with distilled water which was decanted. The Dowex was then soaked for 1 hour in 0.1M HCl to make a slurry. The Dowex slurry was loaded into a glass column to make a 35 ml bed. The resin bed was rinsed with 3 bed volumes of distilled water. After washing, the pH of the Dowex slurry was 2.4. The effluent from the Sephadex step above was loaded onto the column at rate of 2 ml/min. The Dowex column was then eluted with phosphate buffered saline with pH adjusted to 4.22 with HCL at a flow rate of 1.9 ml/min for 90 minutes. The effluent and eluent solutions were collected individually and analyzed for mass balance and protein content. Mass balance demonstrated that 45.5% of the loaded solid (0.068 gm) was in the eluent solution and 54.5% (0.082 gm) was in the effluent solution. The effluent was evaporated using a rotary evaporator and the final turmerin fraction product was oven dried.

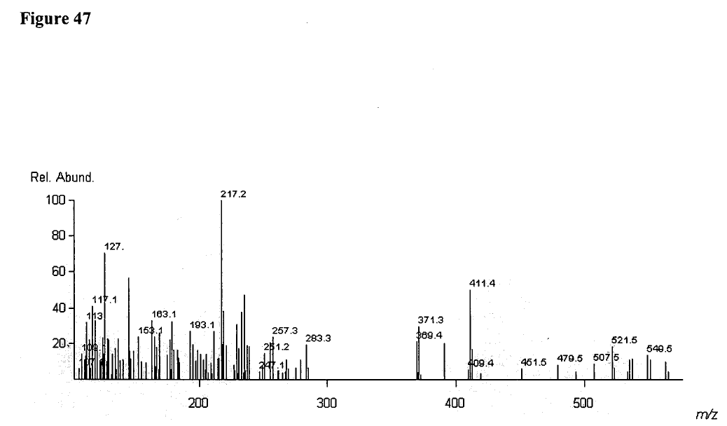

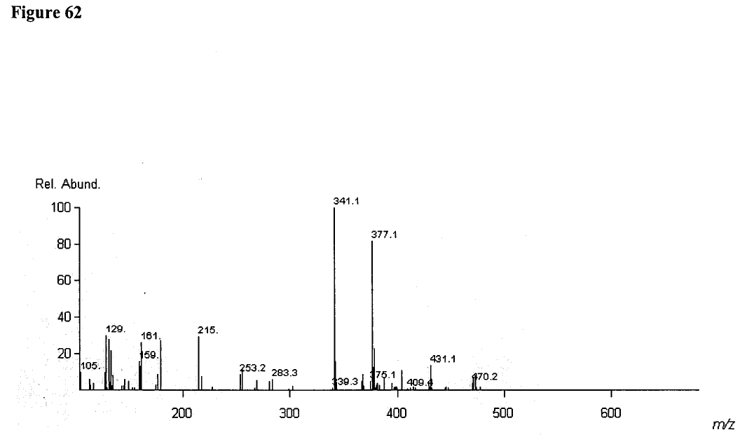

All of the samples from Steps 6 and 7 were analyzed by UV spectrometer, Bradford protein analysis and mass balance. The results of these analyses are shown in Table 21. UV spectrum results are documented in FIG. 8. DART mass spectroscopy chromatograms appear in FIGS. 47 and 62.

TABLE 21
Protein process yield in each step and Bradford analysis results.

| | | | | | Bradford | | BSA eq. | BSA eq. |
|---|---|---|---|---|---|---|---|---|
| | | Mass | Yield | C | abs @ | BSA eq. | in solution | yield |
| No | Sample | (g) | (%) | (mg/ml) | 595 nm | (mg/ml) | (g) | (%) |
| 1 | Crude extract | 3.1 | 9.9 | 0.731 | 0.678 | 0.19 | 0.82 | 2.73 |
| 2 | 60% EtOH | 1.8 | 6.0 | 0.5 | 0.551 | 0.01 | 0.04 | 0.14 |
| | precipitate | | | | | | | |
| 3 | 60% EtOH | 1.3 | 4.2 | 0.5 | 0.579 | 0.05 | 0.13 | 0.44 |
| | solution | | | | | | | |
| 4 | Sephadex | 1.11 | 3.7 | 0.5 | 0.552 | 0.01 | 0.03 | 0.09 |
| | effluent | | | | | | | |
| 5 | Dowex | 0.605 | 2.0 | 0.5 | 0.564 | 0.03 | 0.04 | 0.12 |
| | effluent | | | | | | | |
| 6 | Dowex eluent | 0.505 | 1.7 | 0.5 | 0.547 | 0.01 | 0.01 | 0.02 |

### Example 9

The following ingredients are mixed for the formulation:

| | |
|---|---|
| Extract of curcuma longa L. | 150.0 mg |
| Essential oil Fraction (30 mg, 20% dry weight) | |
| Curcuminoid Fraction (60 mg, 40% dry weight) | |
| Curcuminoid Purity 97% | |
| Curcuminoid Profile | |
| Curcuma 86.2% | |
| Demethoxycurcuma 11.2% | |
| Bisdemethoxycurcuma 2.6% | |
| Polysaccharide Fraction (50 mg, 33.3% dry weight) | |
| Turmerin Fraction (15 mg, 10% dry weight) | |
| Stevioside (Extract of Stevia) | 12.5 mg |
| Carboxymethylcellulose | 35.5 mg |
| Lactose | 77.0 mg |
| Total | 275.0 mg |

The novel extract of curcuma longa L. comprises a purified essential oil fraction, curcuminoid fraction, turmerin fraction, and polysaccharide fraction by % mass weight greater than that found in the natural rhizome material or convention extraction products. In addition, the purity of the curcuminoids in the curcuminoid fraction is greater than 95% with curcuma greater than 85% by mass weight of the curcuminoid chemical constituents. The formulations can be made into any oral dosage form and administered daily or to 15 times per day as needed for the physiological and psychological effects desired (enhanced memory and cognition, analgesia, and relief from chronic arthritic, rheumatic and inflammatory disorders) and medical effects (anti-oxidation and free radical scavenging, anti-platelet aggregation and anti-thrombosis, cardiovascular and cerebrovascular disease prevention and treatment, anti-atherosclerosis, anti-hypercholesterolemia, cytoprotection, nervous system protection, neurological degenerative disease such as Alzheimer's and Parkinson's disease prevention and treatment, anti-inflammatory, anti-allergic, immune enhancement, anti-viral, anti-chronic pulmonary disease, hepatic protection and diseases, anti-peptic ulcer disease, anti-viral and anti-HIV, and cancer prophylaxis and treatment).

### Example 10

The following ingredients were mixed for the following formulation:

| | |
|---|---|
| Extract of curcuma longa L. | 150.0 mg |
| Essential Oil Fraction (18 mg, 12% dry weight) | |
| Curcuminoid Fraction (90 mg, 60% dry weight) | |
| Curcuminoid purity 94% | |
| Curcuminoid distribution profile | |
| Curcuma 75.6% | |
| Demethoxycurcuma 19.3% | |
| Bisdemethoxycurcuma 5.1% | |
| Polysaccharide Fraction (30 mg, 20% dry weight) | |
| Turmerin Fraction (12 mg, 8% dry weight) | |
| Turmerin purity 6.6% | |
| Vitamen C | 15.0 mg |
| Sucralose | 35.0 mg |
| Mung Bean Powder 10:1 | 50.0 mg |
| Mocha Flavor | 40.0 mg |
| Chocolate Flavor | 10.0 mg |
| Total | 300.0 mg |

The novel extractions of curcuma longa L. comprise purified novel essential oil, curcuminoid, turmerin, and polysaccharide chemical constituent fractions by % mass weight greater than that found in the natural plant material or conventional extraction products. Note also the profile change in the curcuma species extractions (The essential oil/curcuminoid ratio in the feedstock was 0.97/1 and in the extract is 0.2/1; the essential oil/polysaccharide ratio in the feedstock was 1.1/1 and in the extract 0.6/1; the essential oil/turmerin ratio in the feedstock was 66.4/1 and in the extract 34/1; the curcuminoid/polysaccharide ratio in the feedstock was 1.2/1 and in the extract 2/0/1; the curcuminoid/turmerin ratio in the feedstock was 66.4/1 and in the extract 113/1; and the polysaccharide/turmerin ratio in the feedstock was 59/1 and in the extract was 56/1). Furthermore the curcuminoid distribution has been altered to increase the concentration of curcumin 66% in the natural feedstock plant material to greater than 75% as a % mass weight of the curcuminoids. The formulation can be made into any oral dosage form and administered safely up to 15 times per day as needed for the physiological, psychological and medical effects desired (see Example 1, above).

### Example 11

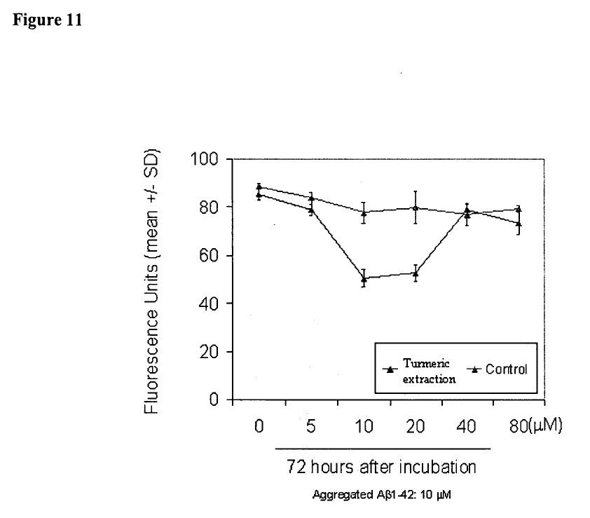

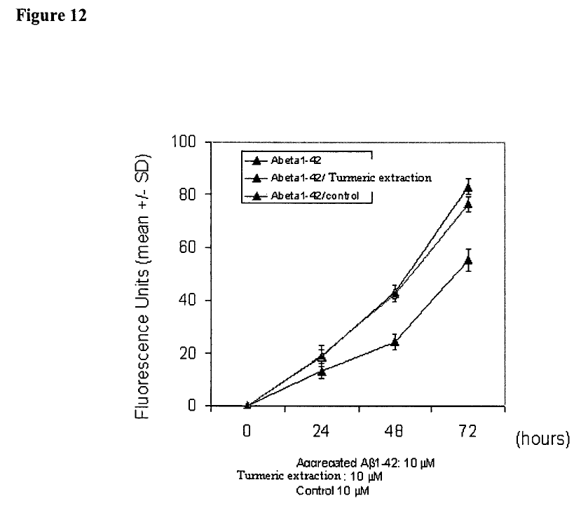

Aggregation Assay—These assays were carried out with the synthetic Aβ₁₋₄₂ peptide incubated with a curcuma extract according to the present invention at varying concentrations from 5 to 80 μM (FIG. 11), or with the curcuma extract and control (at 10 μM) for different time points up to 72 hours (FIG. 12), with aggregation being monitored by the thioflavin T method. The thioflavin T method detects mainly mature β-pleated sheet amyloid fibers. The curcuma extract was an effective inhibitor of Aβ₁₄₂ aggregation in this assay as compared to the control compound. As shown in FIG. 11, the curcuma extract at 10 or 20 μM significantly inhibits Aβ₁₋₄₂ aggregation (P<0.001; ANOVA). Furthermore, FIG. 12 shows data for time-dependent effects of the curcuma extract on Aβ₁₋₄₂ aggregation. In these experiments at 10 μM, curcuma extract incubation shows a time dependent inhibition of aggregation that was significant by 48 hours and increased further at 72 hours of incubation.
Aβ ELISA—In order to examine the effects of the curcuma extract on APP (amyloid precursor protein) cleavage, SweAPP N2a cells were treated with a wide dose-range of each of these compounds for 12 hours. It was found that the curcuma extract reduces Aβ generation (both Aβ₁₋₄₀ and Aβ₁₋₄₂ peptides) in SweAPP N2a cells in a dose-dependent manner (FIG. 12). Most importantly, at a concentration of 10 or 20 μM, the curcuma extract reduces Aβ generation from SweAPP N2a cells by 30 to 38% as compared to untreated cells.

### REFERENCES

1. Asai A & Miyazawa T. J Nutr 131:2932, 2001.
2. Asai A & Miyazawa T. Life Sci 67:2785, 2000.
3. Srinivas L et al. Arch Biochem Biophys 292(2):617, 1992.
4. Lee S K et al. J Environ Pathol Toxicol Oncol 21(2):141, 2002.
5. Gonda R et al. Chem Pharm Bull (Tokyo) 38(2):482, 1990.
6. Kim K I et al. Mol Cells 10(4):392, 2000.
7. Govendarajan V S. CRC Critical Reviews in Food Sci and Nutrition 12(3):200, 1980.
8. Gonda R et al. Chem Pharm Bull 40:185, 1992.
9. Anuchapreeda S et al. Biochem Pharmacol 64:573, 2002.
10. Srinivasan K R. Current Sci 2:311, 1952.
11. Kiuchi F et al. Chem Pharm Bull 41:1640, 1993.
12. Shoba G et al. Planta Med 64:353, 1998.
13. Sharma R A et al. Clin Cancer Res 7:1452, 2001.
14. Lal B et al. Phytother Res 13:318, 1999.
15. Cheng A L et al. Anticancer Res 21:2895, 2001.
16. Sharma R A et al. Clin Cancer Res 7:1894, 2001.
17. Hong C H et al. Planta Med 68(6):545, 2002.
18. Jayaprakasha G K et al. Z Naturforsch [C] 57(9-10):828, 2002.
19. Satoskar R R et al. Int J Clin Pharmocol Toxicol 24:651, 1986.
20. Chandra D & Gupta S S. J Med Res 60:138, 1972.
21. Deodhar S D et al. Ind J Med Res 71:632, 1980.
22. Srivastava V et al. Arzneim.-Forsch./Drug Res 36(1):715, 1986.
23. Srivastava R et al. Thrombosis Res 40:413, 1985.
24. Soni K B & Kuttan R. Ind J Physiol Pharmacol 36(4):273-5 & 239-43, 1992.
25. Cohly M H P et al. Int J Mol Sci 3:985, 2002.
26. Jain J P et al. J Res Ind Med Yoga and Homeo 14:110, 1979.
27. Egan M E et al. Science 304:600, 2004.
28. Dunsmore K E et al. Crit. Care Med 29(11):2199, 2001.
29. Shakai K et al. Chem Pharm Bull 37(10):215, 1989.
30. Lim G P et al. J Neurosci 21:8370, 2001.
31. Natarajan C & Bright J J. J Immunol 168(12):6506, 2002.
32. Shao Z M et al. Int J Cancer 98(2):234, 2002.
33. Kim K I et al. Biosci Biotechnol Biochem 65(11):2369, 2001.
34. Li C J et al. Proc Natl Acad Sci (USA) 90:1839.
35. Phan T T et al. J Trauma 51(5):927, 2001.
36. Williamson E M. Phytomed 8(5):401, 2001.
37. Jovanovic S V et al. J Am Chem Soc 123(13):3064, 2001.
38. Siwak D R et al. Cancer 104:879-890, 2005.

We claim:

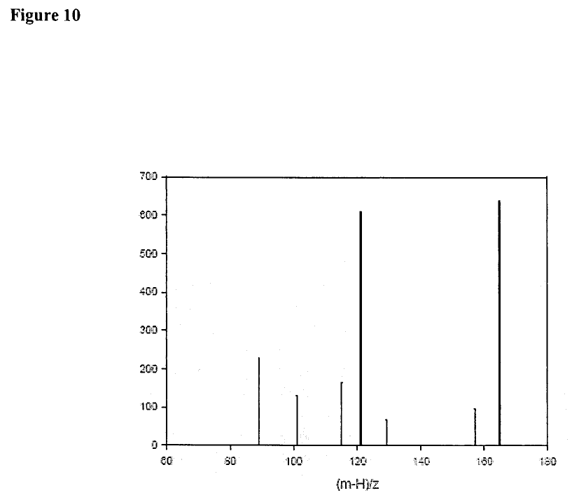

1. A curcuma species extract comprising a fraction having a Direct Analysis in Real Time (DART) mass spectrometry chromatogram of any of FIGS. 9, 10, or 14-78.

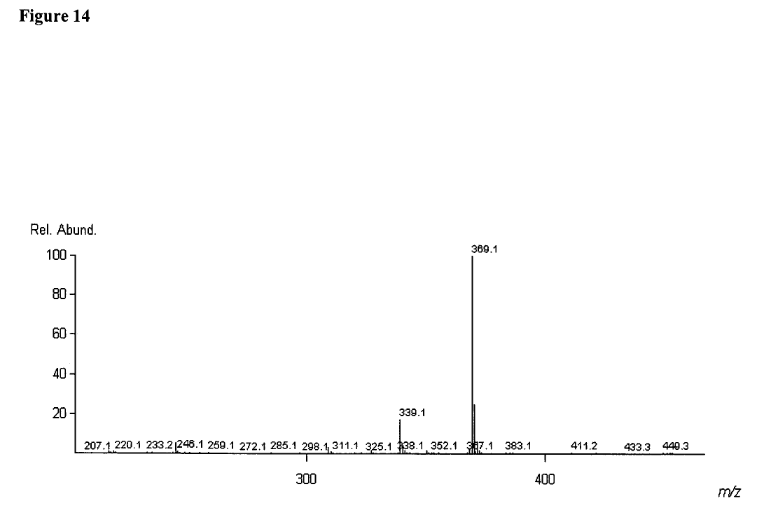

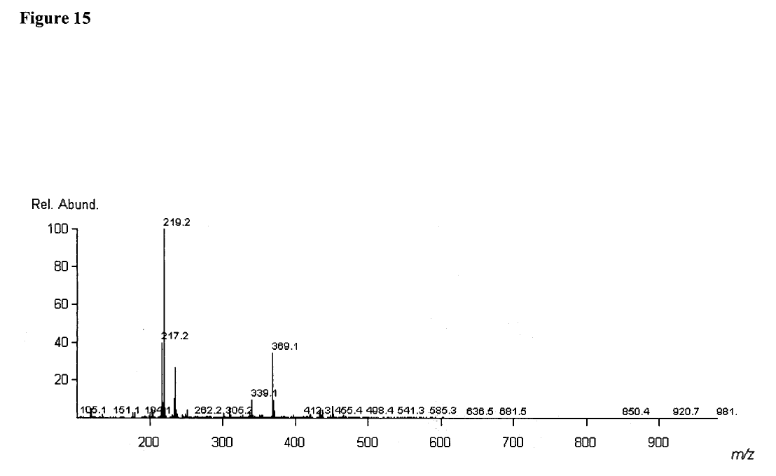

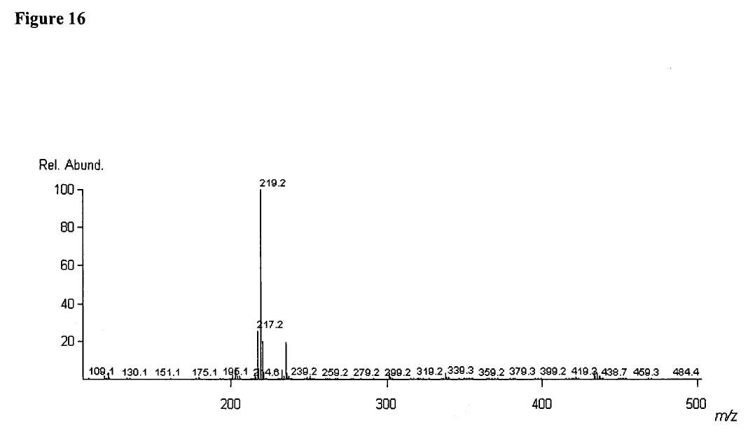

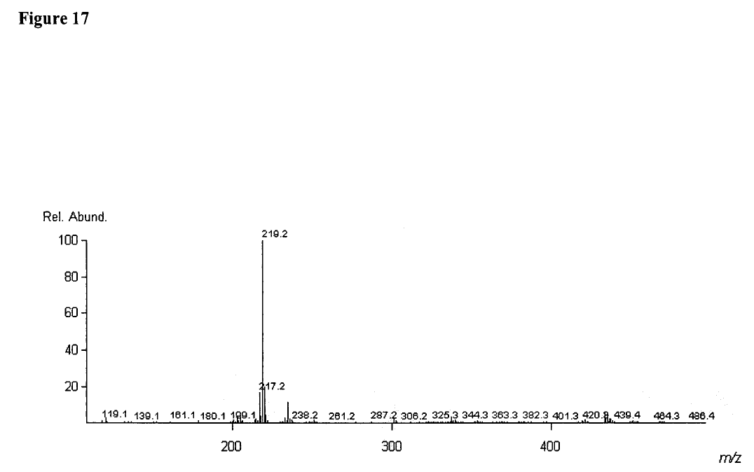

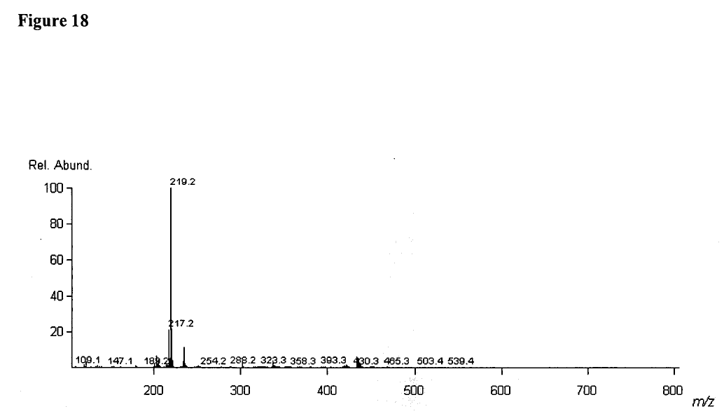

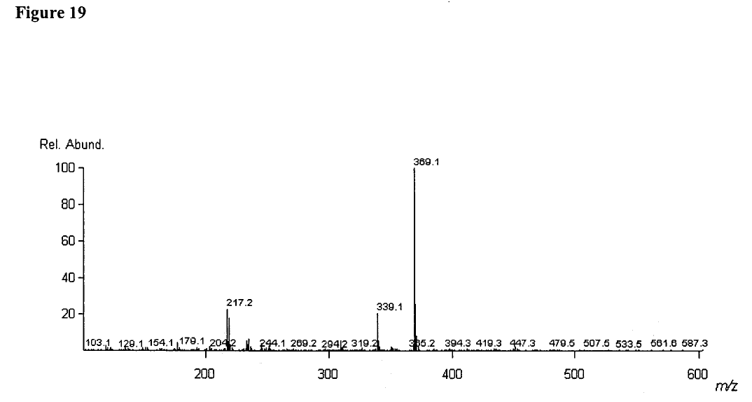

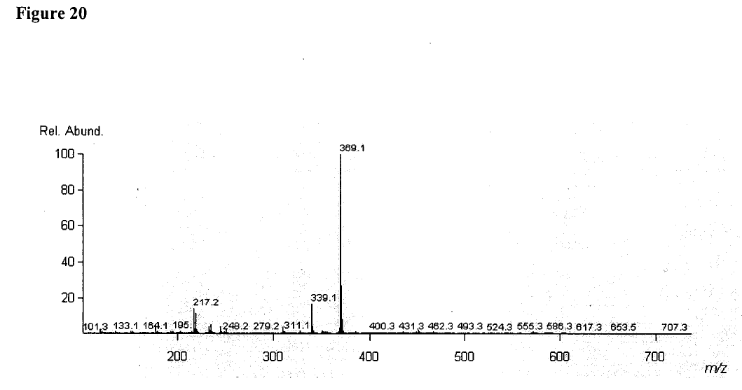

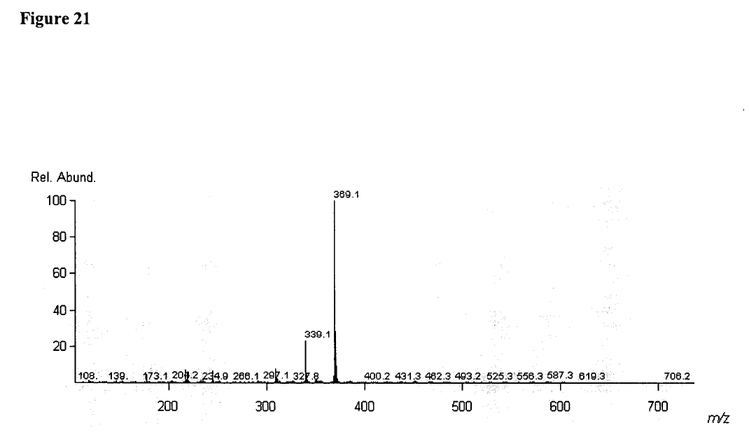

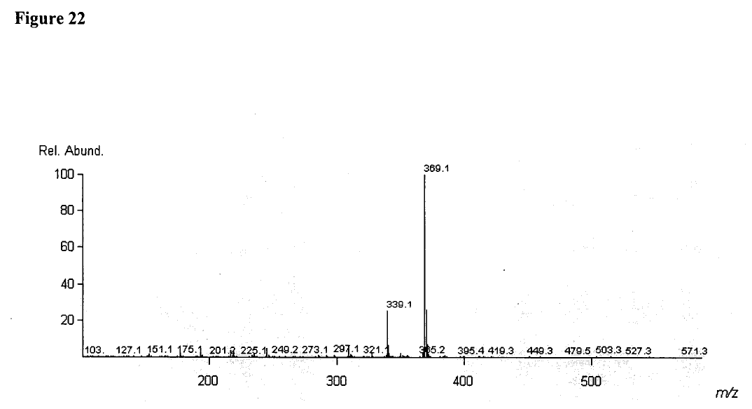

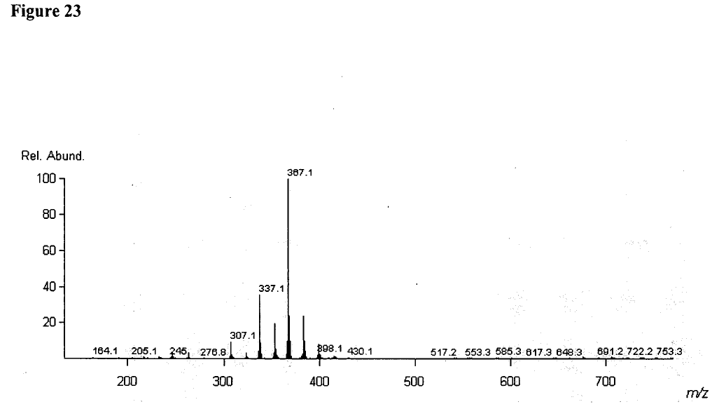

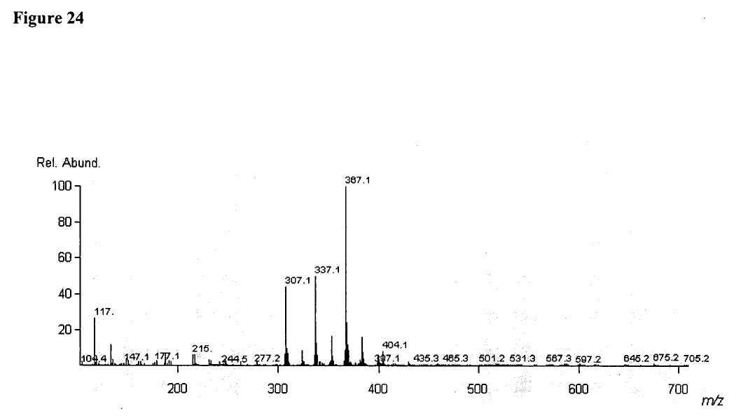

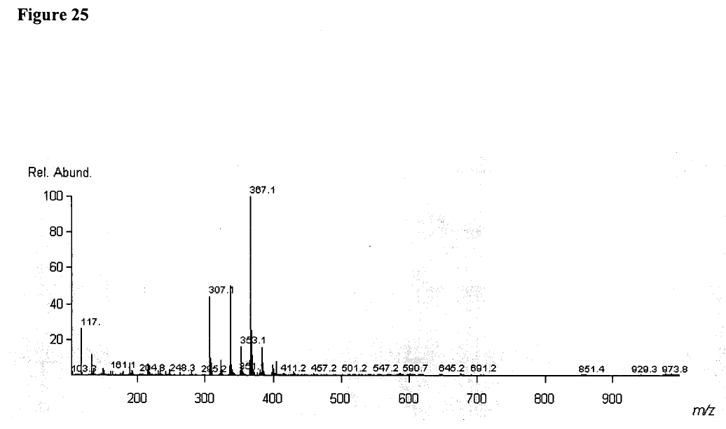

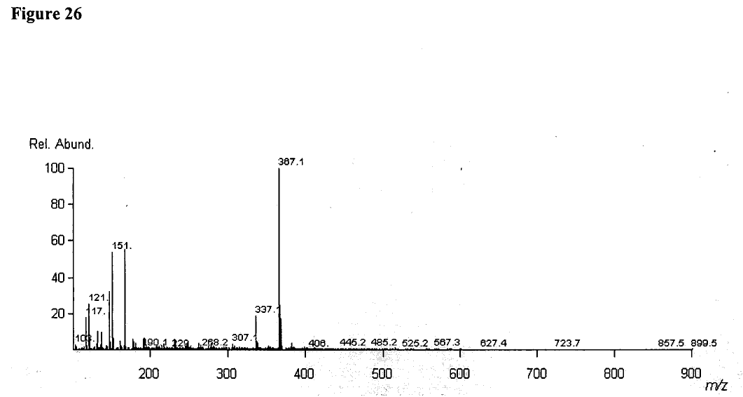

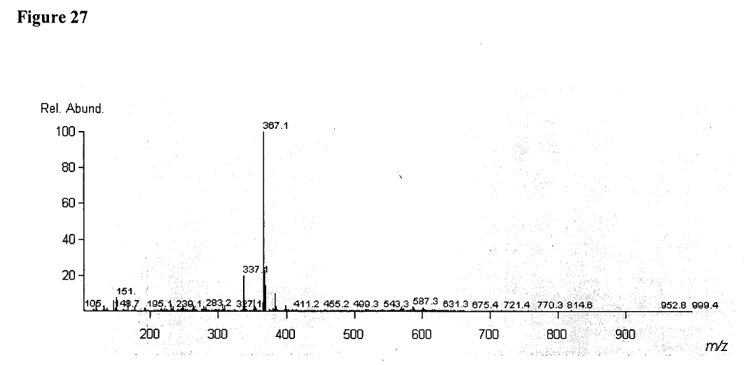

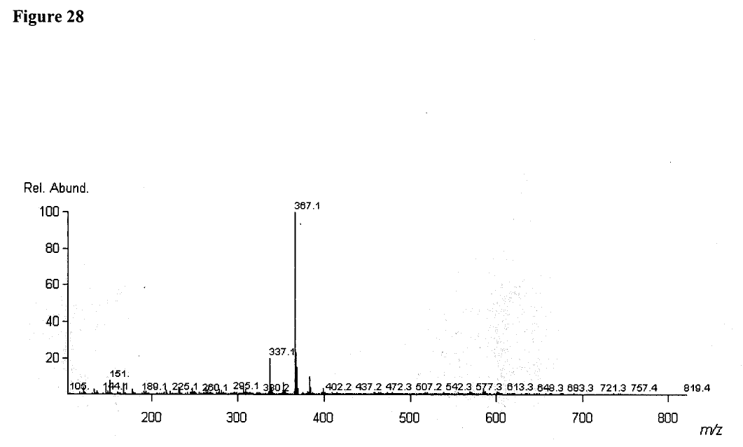

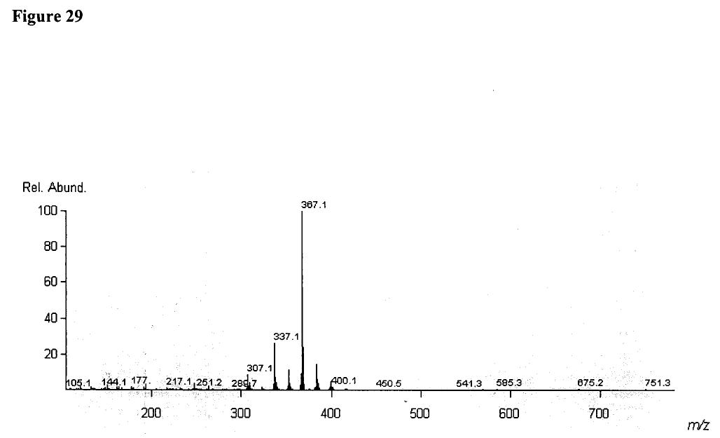

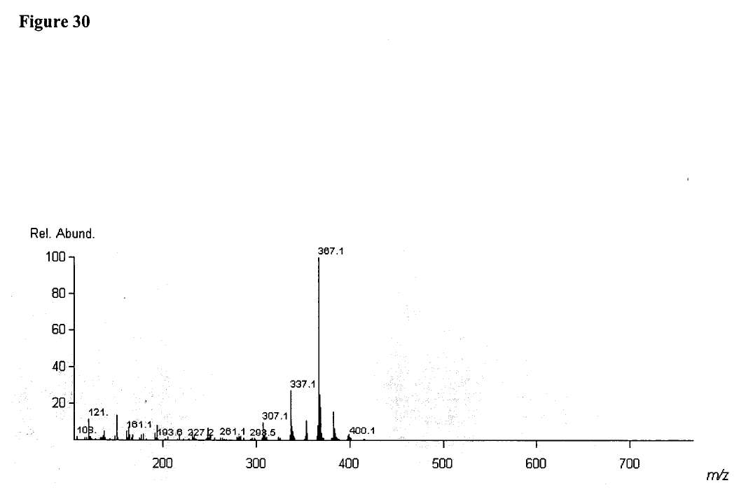

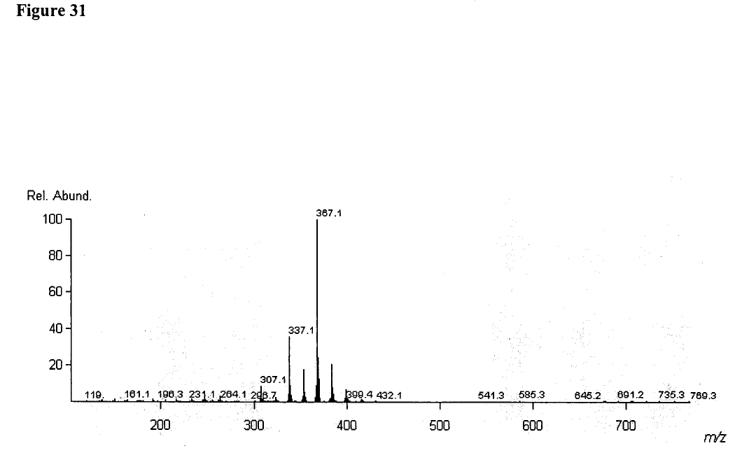

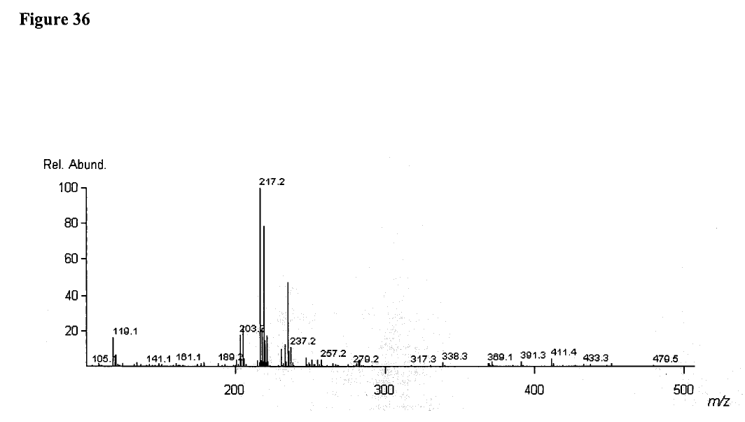

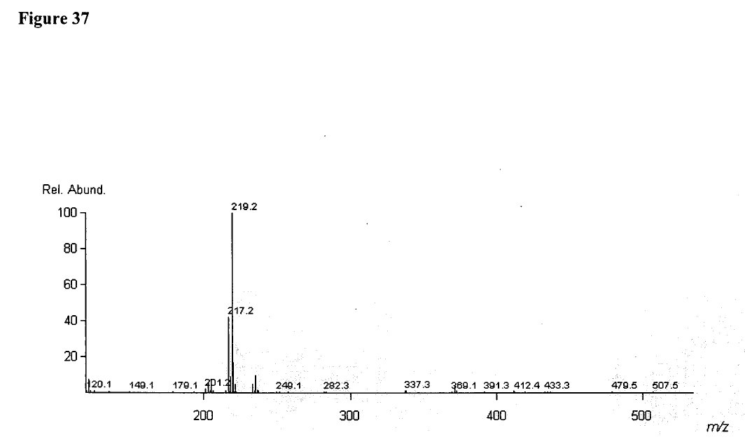

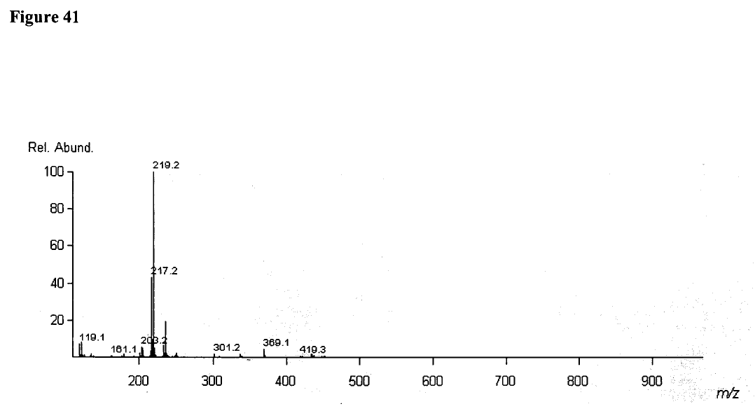

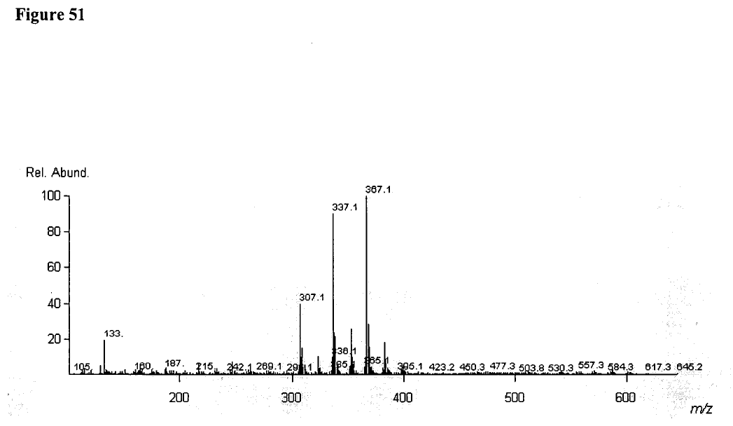

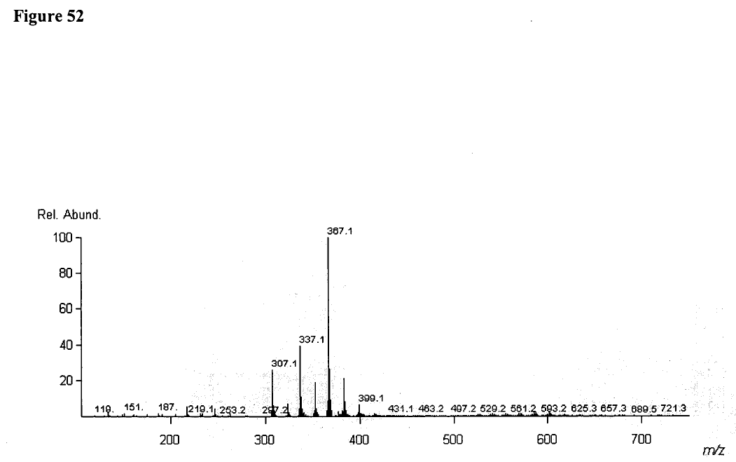

2. The curcuma species extract of claim 1, wherein the fraction has a DART mass spectrometry chromatogram of any of FIGS. 14-31, 36, 37, 41, 51, 52, or 56.

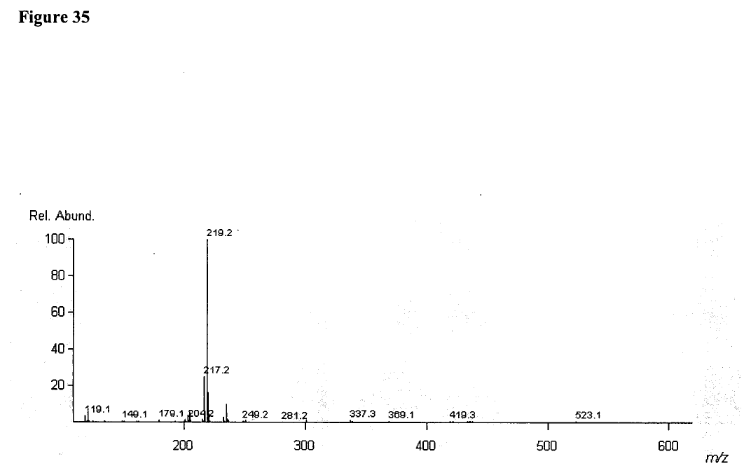

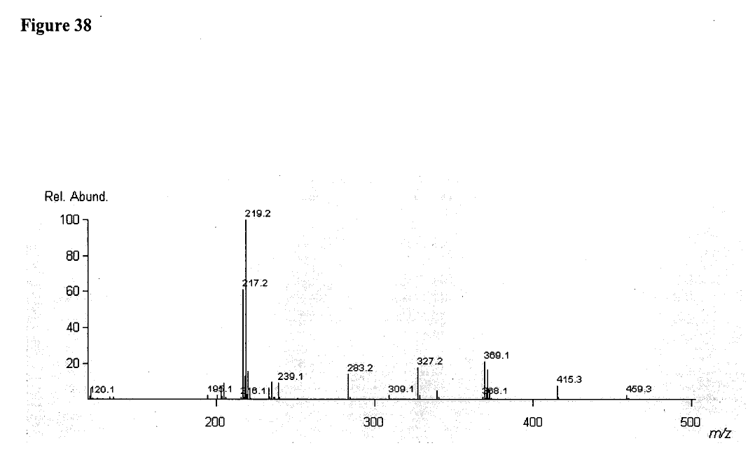

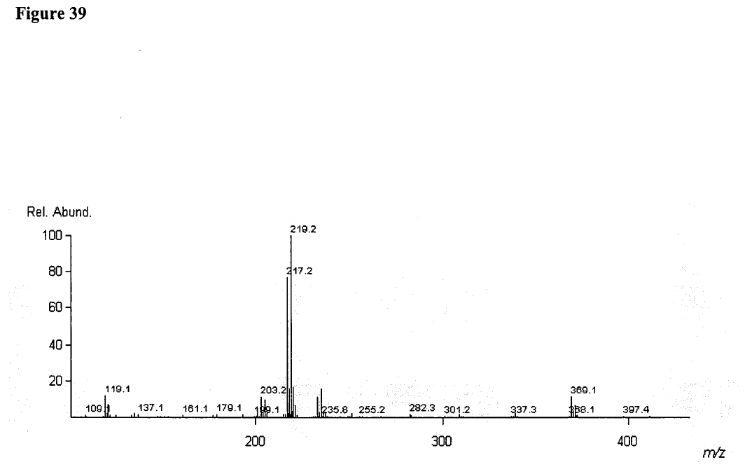

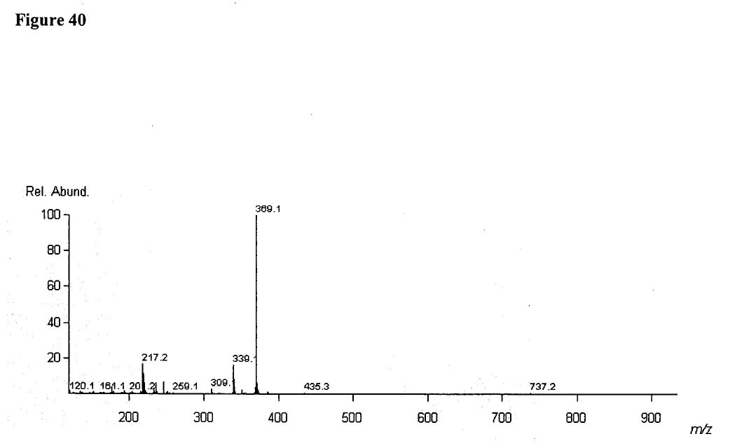

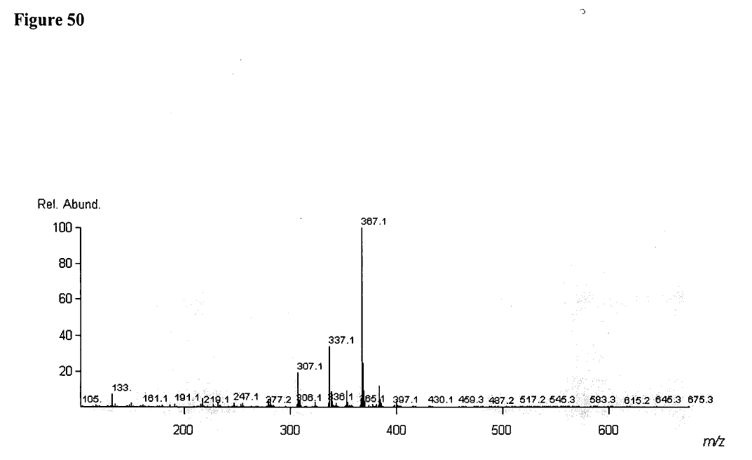

3. The curcuma species extract of claim 1, wherein the fraction has a DART mass spectrometry chromatogram of any of FIGS. 35, 38-40, 50, or 53-55.

4. The curcuma species extract of claim 1, wherein the fraction has a DART mass spectrometry chromatogram of any of FIGS. 9, 10, 42-46, or 57-61.

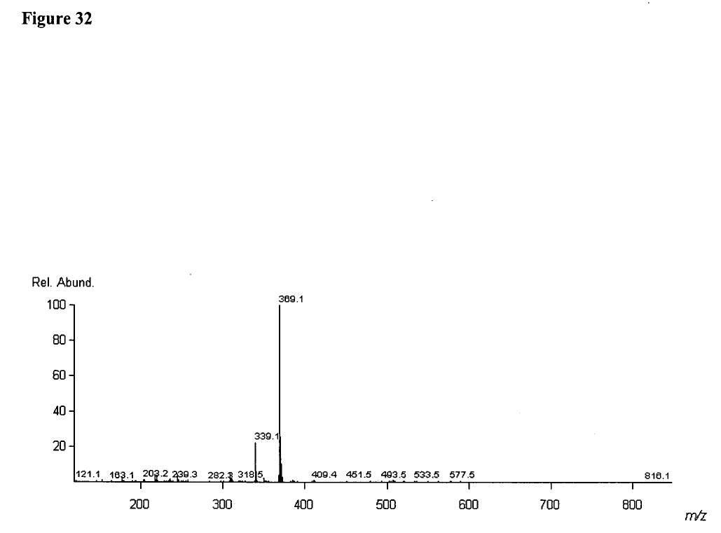

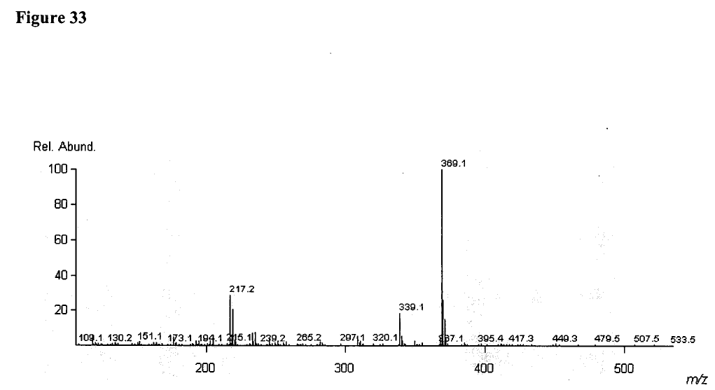

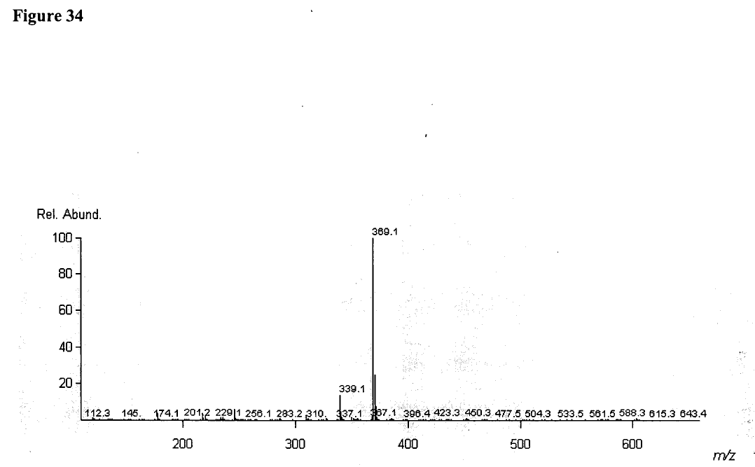

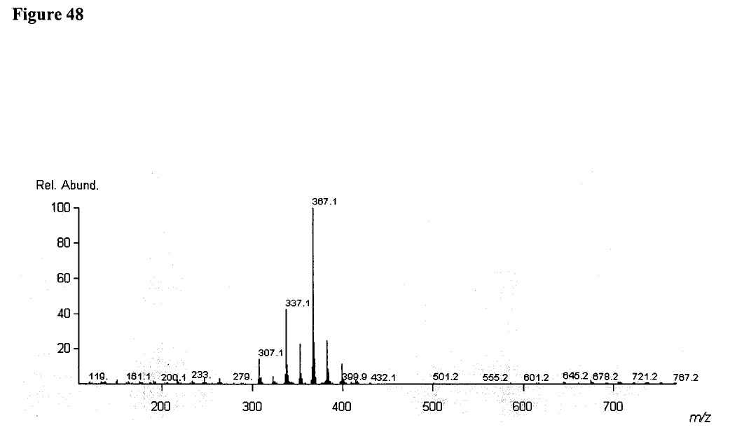

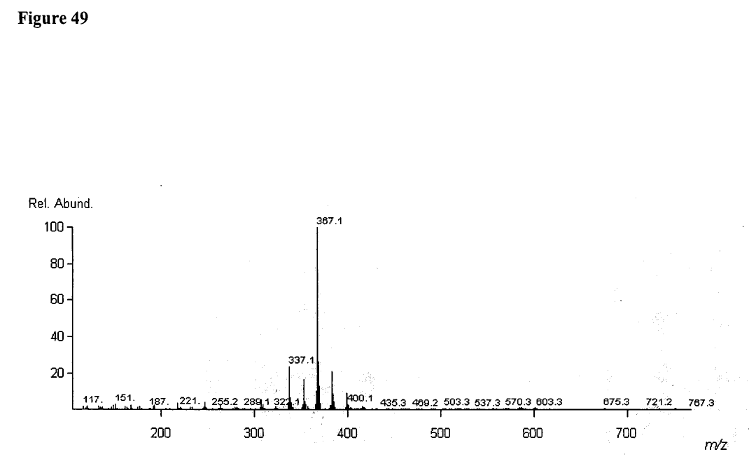

5. The curcuma species extract of claim 1, wherein the fraction has a DART mass spectrometry chromatogram of any of FIGS. 32-34 or 47-49.

6. The curcuma species extract of claim 1, wherein the fraction has a DART mass spectrometry chromatogram of any of FIGS. 63-78.

7. The curcuma species extract of claim 1, wherein the fraction has a DART mass spectrometry chromatogram of FIG. 47 or 62.

8. The curcuma species extract of claim 1, wherein the extract comprises an essential oil fraction having a DART mass spectrometry chromatogram of any of FIGS. 63-78 and a polysaccharide fraction having a DART mass spectrometry chromatogram of any of FIGS. 9, 10, 42-46, or 57-61.

9. The curcuma species extract of claim 1, wherein the extract comprises an essential oil fraction having a DART mass spectrometry chromatogram of any of FIGS. 63-78, a polysaccharide fraction having a DART mass spectrometry chromatogram of any of FIGS. 9, 10, 42-46, or 57-61, and a turmerin fraction having a DART mass spectrometry chromatogram of FIG. 47 or 62.

10. The curcuma species extract of claim 1, wherein the extract comprises a curcuminoid, a turmerone, a polysaccharide, and/or turmerin.

11. The curcuma species extract of claim 10, wherein the curcuminoid is selected from the group consisting of curcumin, tetrahydrocurcumin, demethoxycurcumin, bisdemethoxycurcumin, and combinations thereof.

12. The curcuma species extract of claim 10, wherein the amount of curcuminoid is at least about 75% by weight.

13. The curcuma species extract of claim 10, wherein the turmerone is selected from the group consisting of alpha-turmerone, ar-turmerone, beta-turmerone, and combinations thereof.

14. The curcuma species extract of claim 10, wherein the amount of turmerone is at least 5% by weight.

15. The curcuma species extract of claim 10, wherein the amount of turmerin is at least about 5% by weight.

16. The curcuma species extract of claim 10, wherein the polysaccharide is selected from the group consisting of Ukonan A, Ukonan B, Ukonan C, and a combination thereof.

17. The curcuma species extract of claim 10, wherein the amount of polysaccharide is at least about 5% by weight.

18. Food or medicament comprising the curcuma species extract of claim 1.

19. A method of treating a subject suffering from amyloid plaque aggregation or fibril formation comprising administering to the subject in need thereof an effective amount of the curcuma species extract of claim 1.

20. The method of claim 19, wherein the subject is suffering from Alzheimer's disease.

21. The method of claim 19, wherein the curcuma species extract further comprises a synergistic amount of α- and/or β-boswellic acid and/or its C-acetates.

22. The method of claim 19, wherein the subject is a primate, bovine, ovine, equine, procine, rodent, feline, or canine.

23. The method of claim 19, wherein the subject is a human.

24. A method of preventing amyloid plaque aggregation or fibril formation in tissue comprising contacting the tissue with an effective amount of the curcuma species extract of claim 1.

25. The method of claim 24, wherein the curcuma species extract further comprises a synergistic amount of α- and/or β-boswellic acid and/or its C-acetates.

26. A method of preparing a curcuma species extract having at least one predetermined characteristic comprising: sequentially extracting a curcuma species plant material to yield an essential oil fraction, curcuminoid fraction, polysaccharide fraction, and turmerin fraction by
a. extracting a curcuma species plant material by supercritical carbon dioxide extraction to yield the essential oil fraction and a first residue;
b. extracting either a curcuma species plant material or the first residue from step a) by supercritical carbon dioxide extraction to yield the curcuminoid fraction and a second residue;
c. extracting the second residue from step b) by hot water extraction to yield a polysaccharide solution and then precipitating the polysaccharide with ethanol to yield the polysaccharide fraction and a third residue; and
d. separating from the third residue from step c) by column chromatography the turmerin fraction.

27. The method of claim 26, wherein step a) comprises:
1) loading in an extraction vessel, ground curcuma species plant material;
2) adding carbon dioxide under supercritical conditions;
3) contacting the ground curcuma species plant material and the carbon dioxide for a time; and
4) collecting the essential oil fraction in a collection vessel.

28. The method of claim 27, wherein the supercritical conditions comprise a pressure of from about 250 bar to about 500 bar and a temperature of from about 30° C. to about 80° C.

29. The method of claim 27, wherein extracting conditions for step a) comprise an extraction vessel pressure of from about 250 bar to 500 bar and a temperature of from about 35° C. to about 90° C. and a separator collection vessel pressure of from about 40 bar to about 150 bar and a temperature of from about 20° C. to about 50° C.

30. The method of claim 26, wherein step b) comprises:
1) loading in an extraction vessel, either ground curcuma species plant material or the first residue from step a);
2) adding carbon dioxide under supercritical conditions;
3) contacting the ground curcuma species plant material or first residue from step a) and the carbon dioxide for a time; and
4) collecting the curcuminoid fraction in a fractionation separator collection vessel.

31. The method of claim 30, wherein the extraction conditions for step b) comprise an extraction vessel pressure of from about 350 bar to about 700 bar and a temperature of from about 60° C. to about 95° C. and a separator collection vessel pressure of from about 120 bar to about 220 bar and a temperature of from about 55° C. to about 75° C.

32. The method of claim 26, wherein step c) comprises:
1) contacting the second residue from step b) with a water solution at about 85° C. to about 100° C. for a time sufficient to extract polysaccharides;
2) separating the solid polysaccharides from the solution by ethanol precipitation; and
3) purifying the polysaccharide fraction using column chromatography.

33. The method of claim 26, wherein step d) comprises:
1) passing the third residue from step c) through a resin column for separation of high and low molecular weight molecules; and
2) purifying the higher molecular weight effluent solution using a cation exchange resin column to collect the turmerin fraction from the effluent solution.

34. A curcuma species extract prepared by the method of any of claims 26-33.

35. A curcuma species extract comprising curcumin, tetrahydrocurcumin at 0.1 to 5% by weight of the curcumin, demethoxycurcumin at 10 to 20% by weight of the curcumin, and bisdemethoxycurcumin at 1 to 5% by weight of the curcumin.

36. A curcuma species extract comprising curcumin, tetrahydrocurcumin at 0.1 to 5% by weight of the curcumin, demethoxycurcumin at 15 to 25% by weight of the curcumin, and bisdemethoxycurcumin at 1 to 10% by weight of the curcumin.

37. A curcuma species extract comprising curcumin, tetrahydrocurcumin at 0.1 to 5% by weight of the curcumin, demethoxycurcumin at 20 to 30% by weight of the curcumin, and bisdemethoxycurcumin at 1 to 10% by weight of the curcumin.

38. A curcuma species extract comprising curcumin, demethoxycurcumin at 30 to 40% by weight of the curcumin, and bisdemethoxycurcumin at 5 to 15% by weight of the curcumin.

39. A curcuma species extract comprising curcumin, demethoxycurcumin at 45 to 55% by weight of the curcumin, and bisdemethoxycurcumin at 40 to 50% by weight of the curcumin.

40. A curcuma species extract comprising curcumin, demethoxycurcumin at 15 to 25% by weight of the curcumin, and bisdemethoxycurcumin at 1 to 10% by weight of the curcumin.

41. A curcuma species extract comprising curcumin, tetrahydrocurcumin at 0.1 to 5% by weight of the curcumin, demethoxycurcumin at 20 to 30% by weight of the curcumin, and bisdemethoxycurcumin at 5 to 15% by weight of the curcumin.
